(19) United States
(12) Patent Application Publication
(10) Pub. No.: US 2009/0098145 A1
(43) Pub. Date: Apr. 16, 2009

(54) METHODS AND COMPOSITIONS FOR TREATING OPHTHALMIC CONDITIONS VIA MODULATION OF MEGALIN ACTIVITY

(75) Inventors: Nathan L. MATA, San Diego, CA (US); Yun Han, San Diego, CA (US)

Correspondence Address: WILSON SONSINI GOODRICH & ROSATI, 650 PAGE MILL ROAD, PALO ALTO, CA, 94304-1050, US

(73) Assignee: Sirion Therapeutics, Inc., San Diego, CA (US)

(21) Appl. No.: 11/767,426

(22) Filed: Jun. 22, 2007

Related U.S. Application Data
(60) Provisional application No. 60/805,586, filed on Jun. 22, 2006.

(51) Int. Cl. A61K 39/395; A61K 31/16

(52) U.S. Cl. 424/172.1; 514/613

(57) ABSTRACT

Compounds that cause reversible night blindness may be used to treat ophthalmic conditions associated with the overproduction of waste products that accumulate during the course of the visual cycle. Provided are methods and compositions using such compounds and their derivatives to treat, for example, the macular degenerations and dystrophies or to alleviate symptoms associated with such ophthalmic conditions. Such compounds and their derivatives may be used as single agent therapy or in combination with other agents or therapies.

### CROSS-REFERENCE

This application claims the benefit of U.S. provisional application Ser. No. 60/805,586 filed Jun. 22, 2006, which is incorporated by reference in its entirety.

### BACKGROUND OF THE INVENTION

The vertebrate retina contains two types of photoreceptor cells—rods and cones. Rods are specialized for vision under low light conditions. Cones are less sensitive, provide vision at high temporal and spatial resolutions, and afford color perception. Under daylight conditions, the rod response is saturated and vision is mediated entirely by cones. Both cell types contain a structure called the outer segment comprising a stack of membranous discs. The reactions of visual transduction take place on the surfaces of these discs. The first step in vision is absorption of a photon by an opsin-pigment molecule (rhodopsin), which involves 11-cis to all-trans isomerization of the chromophore. Before light sensitivity can be regained, the resulting all-trans-retinal must be converted back 11-cis-retinal in a multi-enzyme process which takes place in the retinal pigment epithelium (RPE), a monolayer of cells adjacent to the retina.

Currently, treatment options for ophthalmic conditions are limited, especially for ophthalmic conditions involving the retina and/or macula.

### SUMMARY OF THE INVENTION

Described herein are methods and compositions for treating an ophthalmic condition in an eye of a mammal that includes administering to the mammal an effective amount of an agent that modulates the activity of a member of the LDL receptor gene family in the retina and/or retinal pigment epithelium cells in the eye of the mammal.

In one embodiment, the member of the LDL receptor gene family in the retina and/or retinal pigment epithelium cells in the eye is megalin, a megalin-related protein, LRP, or a LRP-related protein. In another embodiment, the member of the LDL receptor gene family in the retina and/or retinal pigment epithelium cells in the eye is megalin, or a megalin-related protein. In a further embodiment, the member of the LDL receptor gene family in the retina and/or retinal pigment epithelium cells in the eye is megalin. In a further embodiment, the member of the LDL receptor gene family in the retina and/or retinal pigment epithelium cells in the eye is LRP, or a LRP-related protein. In a further embodiment, the member of the LDL receptor gene family in the retina and/or retinal pigment epithelium cells in the eye is LRP. In one embodiment, the member of the LDL receptor gene family in the retina and/or retinal pigment epithelium cells in the eye is a megalin-related protein. In a further embodiment, the member of the LDL receptor gene family in the retina and/or retinal pigment epithelium cells in the eye is a LRP-related protein. In another embodiment, the member of the LDL receptor gene family in the retina and/or retinal pigment epithelium cells in the eye is a protein comprising peptide sequences listed in FIG. 3.

In one embodiment, the member of the LDL receptor gene family in the retina and/or retinal pigment epithelium cells in the eye of a mammal is a retinoid binding protein receptor. In another embodiment, the member of the LDL receptor gene family in the retina and/or retinal pigment epithelium cells in the eye of a mammal is a RBP and/or IRBP receptor. In another embodiment, the member of the LDL receptor gene family in the retina and/or retinal pigment epithelium cells in the eye of a mammal is STRA6 or a STRA6-related protein. In another embodiment, the member of the LDL receptor gene family in the retina and/or retinal pigment epithelium cells in the eye of a mammal is STRA6. In another embodiment, the member of the LDL receptor gene family in the retina and/or retinal pigment epithelium cells in the eye of a mammal is a STRA6-related protein.

Members of the LDL receptor gene family are present on the basal membrane and apical membrane of RPE cells in the eye. In some embodiments, the receptors on the basal membrane of RPE cells are not the same as the receptors on the apical membrane of RPE cells in the eye. In some embodiments, the receptors on the basal membrane of RPE cells are the same as the receptors on the apical membrane of RPE cells. In some embodiments, an agent modulates the activity of a member of the LDL receptor gene family on the basal membrane of RPE cells in the eye. In some embodiments, an agent modulates the activity of a member of the LDL receptor gene family on the apical membrane of RPE cells in the eye. In some embodiments, an agent modulates the activity of a member of the LDL receptor gene family on the basal membrane of RPE cells and does not modulate the activity of a member of the LDL receptor gene family on the apical membrane of RPE cells. In some embodiments, an agent modulates the activity of a member of the LDL receptor gene family on the basal membrane of RPE cells and modulates the activity of a member of the LDL receptor gene family on the apical membrane of RPE cells.

In some embodiments, the activity of the member of the LDL receptor gene family is the binding of the member of the LDL receptor gene family to vitamin-binding proteins, lipoproteins, immune- and stress related proteins, steroid hormone binding proteins, hormones and precursors, peptides, enzyme and enzyme inhibitors, albumin, lactoferrin, hemoglobin, odorant-binding protein, transthyretin; drugs and toxins, RAP, calcium (Ca²⁺), or cytochrome c.

In some embodiments, the activity of the member of the LDL receptor gene family is the binding of the member of the LDL receptor gene family to vitamin-binding proteins, carrier proteins, lipoproteins, immune- and stress related proteins, steroid hormone binding proteins, hormones and precursors, peptides, enzyme and enzyme inhibitors, albumin, lactoferrin, hemoglobin, odorant-binding protein, transthyretin; drugs and toxins, RAP, calcium (Ca²⁺), or cytochrome c.

In some embodiments, the activity of the member of the LDL receptor gene family is the binding of the member of the LDL receptor gene family to vitamin-binding proteins, lipoproteins, immune- and stress related proteins, steroid hormone binding proteins, hormones and precursors, peptides, enzyme and enzyme inhibitors, albumin, lactoferrin, hemoglobin, odorant-binding protein, transthyretin; polybasic drugs and toxins, RAP, calcium (Ca²⁺), or cytochrome c.

In one embodiment, the activity of the member of the LDL receptor gene family is the binding of the member of the LDL receptor gene family to retinol, retinal, a retinol-RBP complex, a retinol-RBP-TTR complex, an interphotoreceptor retinoid binding protein (IRBP), a retinol-IRBP complex, a retinal-IRBP complex, transcobalamin-vitamin B12, transcobalamin-vitamin B12 binding protein, vitamin-D-binding protein, apolipoprotein B, apolipoprotein E, apolipoprotein J/clusterin, apolipoprotein H; immunoglobulin light chains, PAP-1, β2-microglobulin; sex hormone binding protein-estrogens, androgen binding protein-androgens; parathyroid hormone, insulin, epidermal growth factor, prolactin, thyroglobulin; plasminogen activator inhibitor-1 (PAI-1), urokinase-PAI-1, tPA-PAI-1, pro-urokinase, lipoprotein lipase, plasminogen, β-amylase, β1-microglobulin, lysozyme; albumin, lactoferrin, hemoglobin, odorant-binding protein, transthyretin; aminoglycosides, polymyxin B, aprotinin, trichosanthin, gentamicin; RAP, Ca²⁺, or cytochrome c.

In one embodiment, the activity of a member of the LDL receptor gene family is the binding of the member of the LDL receptor gene family to drugs and toxins. In one embodiment, the activity of a member of the LDL receptor gene family is the binding of the member of the LDL receptor gene family to polybasic drugs and toxins. In another embodiment, the activity of the member of the LDL receptor gene family is the binding of the member of the LDL receptor gene family to cationic drugs and toxins. In another embodiment, the activity of the member of the LDL receptor gene family is the binding of the member of the LDL receptor gene family to cationic amine drugs and toxins. In one embodiment, the activity of the member of the LDL receptor gene family is the binding of the member of the LDL receptor gene family to antibacterials, antipsychotics, antidepressants, antiarrythmics, antianginals, anorexic agents, or cholesterol-lowering agents

In one embodiment, the activity of a member of the LDL receptor gene family is the binding of a member of the LDL receptor gene family to aminoglycosides. In another embodiment, the activity of the member of the LDL receptor gene family is the binding of a member of the LDL receptor gene family to arbekacin, gentamicin, kanamycin, neomycin, paramycin, ribostamycin, lividomycin, amikacin, dibekacin, butakacin, tobramycin, streptomycin, dihydrostroptomycin, sisomicin, verdamicin, netilmicin, or butikacin. In another embodiment, the activity of the member of the LDL receptor gene family is the binding of a member of the LDL receptor gene family to arbekacin, gentamicin, or kanamycin. In another embodiment, the activity of the member of the LDL receptor gene family is the binding of a member of the LDL receptor gene family to gentamicin.

In another embodiment, the activity of a member of the LDL receptor gene family is the binding of a member of the LDL receptor gene family to antimalarials, antibiotic drugs, antituberculosis drugs, antifingal drugs, CNS drugs, cardiovascular drugs, antineoplastic drugs, dermatological drugs, anti-inflammatory drugs, immunomodulator drugs, oral contraceptives, hormones, deferoxamine, niacin, warfarin, or sympathomimetic drugs.

In another embodiment, the activity of a member of the LDL receptor gene family is the binding of a member of the LDL receptor gene family to chloroquine, quinine, aminoglycosides, sparsomycin, clioquinol, ethambutol, miconazole, phenothiazines, chlorpromazine, amitriptyline, lysergide, nifedipine, amiodarone, 5-fluorouracil, tamoxifen, carmustine, chlorambucil, cis-platinum, mitotane, nitrogen mustard, nitroso ureas, vinblastine, vincristine, doxorubicin, etretinate, canthaxanthin, isotretinoin, corticosteroids, ibuprofen, indomethacin, phenylbutazone, tilorone (antiviral) alpha interferon, oral contraceptives, clomiphene, deferoxamine, niacin, warfarin, dipivefrin, phenylephrine, or epinephrine.

In another embodiment, the activity of the LDL receptor gene family member is the binding of the member of the LDL receptor gene family to vitamin-binding proteins. In another embodiment, the activity of the LDL receptor gene family member is the binding of the member of the LDL receptor gene family to retinoid binding proteins. In a further embodiment, the activity of the member of the LDL receptor gene family is the binding of the member of the LDL receptor gene family to retinol, RBP, a retinol-RBP complex, a retinol-RBP-TTR complex, IRBP, or a retinol-IRBP complex. In a further embodiment, the activity of the member of the LDL receptor gene family is the binding of the member of the LDL receptor gene family to retinol, a retinol-RBP complex, or a retinol-RBP-TTR complex. In a further embodiment, the activity of the member of the LDL receptor gene family is the binding of the member of the LDL receptor gene family to IRBP or a retinol-IRBP complex. In a further embodiment, the activity of the member of the LDL receptor gene family is the binding of the member of the LDL receptor gene family to IRBP, a retinol-IRBP complex, or a retinal-IRBP complex.

In one embodiment, the activity of the member of the LDL receptor gene family is the trancytosis of vitamin-binding proteins, lipoproteins, immune- and stress related proteins, steroid hormone binding proteins, hormones and precursors, peptides, enzyme and enzyme inhibitors, albumin, lactoferrin, hemoglobin, odorant-binding protein, transthyretin; drugs and toxins, RAP, calcium (Ca 2+), or cytochrome c.

In one embodiment, the activity of the member of the LDL receptor gene family is the trancytosis of vitamin-binding proteins, lipoproteins, immune- and stress related proteins, steroid hormone binding proteins, hormones and precursors, peptides, enzyme and enzyme inhibitors, albumin, lactoferrin, hemoglobin, odorant-binding protein, transthyretin; polybasic drugs and toxins, RAP, calcium (Ca 2+), or cytochrome c.

In another embodiment, the activity of the member of the LDL receptor gene family is the transcytosis of retinol, a retinol-RBP complex, a retinol-RBP-TTR complex, an interphotoreceptor retinoid binding protein (IRBP), a retinol-IRBP complex, transcobalamin-vitamin B12, transcobalamin-vitamin B 12 binding protein, vitamin-D-binding protein, apolipoprotein B, apolipoprotein E, apolipoprotein J/clusterin, apolipoprotein H; immunoglobulin light chains, PAP-1, β2-microglobulin; sex hormone binding protein-estrogens, androgen binding protein-androgens; parathyroid hormone, insulin, epidermal growth factor, prolactin, thyroglobulin; plasminogen activator inhibitor-1 (PAI-1), urokinase-PAI-1, tPA-PAI-1, pro-urokinase, lipoprotein lipase, plasminogen, β-amylase, β1-microglobulin, lysozyme; albumin, lactoferrin, hemoglobin, odorant-binding protein, transthyretin; aminoglycosides, polymyxin B, aprotinin, trichosanthin, gentamicin; RAP, Ca²⁺, or cytochrome c.

In one embodiment, the activity of a member of the LDL receptor gene family is the transcytosis of drugs and toxins. In one embodiment, the activity of a member of the LDL receptor gene family is the transcytosis of polybasic drugs and toxins. In another embodiment, the activity of the member of the LDL receptor gene family is the transcytosis of cationic drugs and toxins. In another embodiment, the activity of the member of the LDL receptor gene family is the transcytosis of cationic amine drugs and toxins. In one embodiment, the activity of the member of the LDL receptor gene family is the transcytosis of antibacterials, antipsychotics, antidpressants, antiarrythmics, antianginals, anorexic agents, or cholesterol-lowering agents.

In one embodiment, the activity of a member of the LDL receptor gene family is the transcytosis of aminoglycosides. In another embodiment, the activity of the member of the LDL receptor gene family is the transcytosis of arbekacin, gentamicin, kanamycin, neomycin, paramycin, ribostamycin, lividomycin, amikacin, dibekacin, butakacin, tobramycin, streptomycin, dihydrostroptomycin, sisomicin, verdamicin, netilmicin, or butikacin. In another embodiment, the activity of the member of the LDL receptor gene family is the transcytosis of arbekacin, gentamicin, or kanamycin. In another embodiment, the activity of the member of the LDL receptor gene family is the transcytosis of gentamicin.

In another embodiment, the activity of a member of the LDL receptor gene family is the transcytosis of antimalarials, antibiotic drugs, antituberculosis drugs, antifungal drugs, CNS drugs, cardiovascular drugs, antineoplastic drugs, dermatological drugs, anti-inflammatory drugs, immunomodulator drugs, oral contraceptives, hormones, deferoxamine, niacin, warfarin, or sympathomimetic drugs. In another embodiment, the activity of a member of the LDL receptor gene family is the transcytosis of antibiotic drugs.

In another embodiment, the activity of a member of the LDL receptor gene family is the transcytosis of chloroquine, quinine, aminoglycosides, sparsomycin, clioquinol, ethambutol, miconazole, phenothiazines, chlorpromazine, amitriptyline, lysergide, nifedipine, amiodarone, 5-fluorouracil, tamoxifen, carmustine, chlorambucil, cis-platinum, mitotane, nitrogen mustard, nitroso ureas, vinblastine, vincristine, doxorubicin, etretinate, canthaxanthin, isotretinoin, corticosteroids, ibuprofen, indomethacin, phenylbutazone, tilorone (antiviral) alpha interferon, oral contraceptives, clomiphene, deferoxamine, niacin, warfarin, dipivefrin, phenylephrine, or epinephrine.

In one embodiment, the activity of the member of the LDL receptor gene family is the transcytosis of retinol, a retinol-RBP complex, a retinol-RBP-TTR complex, or a retinol-IRBP complex.

In one embodiment, the transcytosis is exocytosis. In another embodiment, the transcytosis is endocytosis.

In another embodiment, the activity of the member of the LDL receptor gene family is the transport across an epithelium of at least one retinal pigment epithelium cell of retinol, a retinol-RBP complex, a retinol-RBP-TTR complex, or a retinol-IRBP complex.

In one embodiment, the agent increases the activity of the member of the LDL receptor gene family. In another embodiment, the agent decreases the activity of the member of the LDL receptor gene family.

In one embodiment, the agent binds to the member of the LDL receptor gene family on the basal membrane of the retinal pigment epithelial cells. In another embodiment, the agent binds to the member of the LDL receptor gene family on the apical membrane of the retinal pigment epithelium cells.

In one embodiment, the agent binds retinol-binding protein. In another embodiment, the agent binds to transthyretin. In another embodiment, the agent binds to interphotoreceptor retinoid binding protein (IRBP).

In one embodiment, the agent modulates the expression of the member of the LDL receptor gene family in the retina and/or retinal pigment epithelium cells. In other embodiments, the agent decreases the expression of the member of the LDL receptor gene family in the retina and/or retinal pigment epithelium cells. In other embodiments, the agent increases the expression of the member of the LDL receptor gene family in the retina and/or retinal pigment epithelium cells.

In one embodiment, the agent is selected from among an antibody, a polypeptide, a nucleic acid, a polynucleic acid, a polymer, receptor associated protein (RAP) (a type of chaperone that is especially designed to assist in the biosynthesis and intracellular transport of endocytic receptors) or fragments thereof, a low molecular weight organic compound, vitamin-binding proteins, lipoproteins, immune- and stress related proteins, steroid hormone binding proteins, hormones and precursors, peptides, enzyme and enzyme inhibitors, albumin, lactoferrin, hemoglobin, odorant-binding protein, transthyretin; polybasic drugs and toxins, RAP, calcium (Ca 2+), calcium scavengers, reducing agents and cytochrome c. In one embodiment, the agent is selected from among an antibody, a polypeptide, a nucleic acid, a polynucleic acid, a polymer, receptor associated protein (RAP) or fragments thereof, a low molecular weight organic compound, vitamin-binding proteins, lipoproteins, immune- and stress related proteins, steroid hormone binding proteins, hormones and precursors, peptides, enzyme and enzyme inhibitors, albumin, lactoferrin, hemoglobin, odorant-binding protein, transthyretin; drugs and toxins, RAP, calcium (Ca 2+), calcium scavengers, reducing agents and cytochrome c. In another embodiment, the agent is an antibody, a polypeptide, a nucleic acid, a polynucleic acid, a polymer, receptor associated protein (RAP) or fragments thereof, a low molecular weight organic compound, retinol, a retinol-RBP complex, a retinol-RBP-TTR complex, an interphotoreceptor retinoid binding protein (IRBP), a retinol-IRBP complex, transcobalamin-vitamin B12, transcobalamin-vitamin B12 binding protein, vitamin-D-binding protein, apolipoprotein B, apolipoprotein E, apolipoprotein J/clusterin, apolipoprotein H; immunoglobulin light chains, PAP-1, β2-microglobulin; sex hormone binding protein-estrogens, androgen binding protein-androgens; parathyroid hormone, insulin, epidermal growth factor, prolactin, thyroglobulin; plasminogen activator inhibitor-1 (PAI-1), urokinase-PAI-1, tPA-PAI-1, pro-urokinase, lipoprotein lipase, plasminogen, β-amylase, β1-microglobulin, lysozyme; albumin, lactoferrin, hemoglobin, odorant-binding protein, transthyretin; aminoglycosides, polymyxin B, aprotinin, trichosanthin, gentamicin; RAP, RAP fragments, Ca 2+, calcium scavengers, reducing agents or cytochrome c.

In one embodiment, the agent is an antibody. In another embodiment, the agent is a polypeptide. In another embodiment, the agent is a nucleic acid. In another embodiment, the agent is a polynucleic acid. In another embodiment, the agent is a polymer. In another embodiment, the agent is an aminoglycoside or derivative thereof. In another embodiment, the agent is RAP or fragments thereof. In further embodiment, the agent is a low molecular weight organic compound.

In another embodiment, the agent is a domain of a member of the LDL receptor gene family. In another embodiment, the agent is a domain of megalin. In another embodiment, the agent is a fragment of a retinoid binding protein. In another embodiment, the agent is a fragment of megalin.

In one embodiment, the effective amount of the agent is systemically administered to the mammal. In another embodiment, the effective amount of the agent is administered orally to the mammal. In another embodiment, the effective amount of the agent is intravenously administered to the mammal. In a further embodiment, the effective amount of the agent is ophthalmically administered to the mammal. In a further embodiment, the effective amount of the agent is administered by iontophoresis. In another embodiment, the effective amount of the agent is administered by injection to the mammal.

In one embodiment, the mammal is a human.

In another embodiment, a method for treating an ophthalmic condition in an eye of a mammal that includes administering to the mammal an effective amount of an agent that modulates the activity of a member of the LDL receptor gene family in the retina and/or retinal pigment epithelium cells in the eye of the mammal includes multiple administrations of the effective amount of the agent. In another embodiment, the time between multiple administrations is at least one week. In another embodiment, the time between multiple administrations is at least one day. In a further embodiment, the compound is administered to the mammal on a daily basis.

In one embodiment, the method further includes administering at least one additional agent selected from the group consisting of an inducer of nitric oxide production, an anti-inflammatory agent, a physiologically acceptable antioxidant, a physiologically acceptable mineral, a negatively charged phospholipid, a carotenoid, a statin, an anti-angiogenic drug, a matrix metalloproteinase inhibitor, 13-cis-retinoic acid, or a compound having the structure of Formula (A):

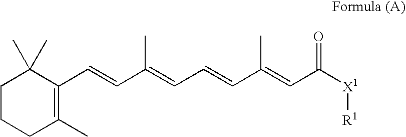

wherein

X¹ is selected from the group consisting of NR², O, S, CHR²;

R¹ is (CHR²)ₓ-L¹-R³, wherein
x is 0, 1, 2, or 3; L¹ is a single bond or —C(O)—;
R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and
R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle.

In one embodiment, compounds of Formula (A) are with a proviso that that R³ is not H when both x is 0 and L¹ is a single bond; or an active metabolite, or a pharmaceutically acceptable prodrug or solvate thereof. In one embodiment, the additional agent is an inducer of nitric oxide production. In one embodiment, the inducer of nitric oxide production is selected from among citrulline, ornithine, nitrosated L-arginine, nitrosylated L-arginine, nitrosated N-hydroxy-L-arginine, nitrosylated N-hydroxy-L-arginine, nitrosated L-homoarginine and nitrosylated L-homoarginine.

In one embodiment, the additional agent is an anti-inflammatory agent. In another embodiment, the additional agent is an anti-inflammatory agent selected from among a non-steroidal anti-inflammatory drug, a lipoxygenase inhibitor, prednisone, dexamethasone, and a cyclooxygenase inhibitor.

In one embodiment, the additional agent is at least one physiologically acceptable antioxidant. In another embodiment, the additional agent is a physiologically acceptable antioxidant selected from among vitamin C, vitamin E, beta-carotene, coenzyme Q, and 4-hydroxy-2,2,6,6-tetramethylpiperadine-N-oxyl.

In one embodiment, the additional agent is at least one physiologically acceptable mineral. In another embodiment, the additional agent is a physiologically acceptable mineral selected from among a zinc (II) compound, a Cu(II) compound, and a selenium (II) compound.

In one embodiment, the additional agent is a negatively charged phospholipid. In another embodiment, the negatively charged phospholipid is phosphatidylglycerol.

In another embodiment, the additional agent is a carotenoid. In another embodiment, the additional agent is a carotenoid selected from among lutein and zeaxanthin.

In another embodiment, the additional agent is a statin. In another embodiment, the additional agent is a statin selected from among rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium, and dihydrocompactin.

In one embodiment, the additional agent is an anti-angiogenic drug. In one embodiment, the additional agent is an anti-angiogenic drug selected from among Rhufab V2, tryptophanyl-tRNA synthetase, an anti-VEGF pegylated aptamer, squalamine, anecortave acetate, Combretastatin A4 Prodrug, Macugen™, mifepristone, subtenon triamcinolone acetonide, intravitreal crystalline triamcinolone acetonide, AG3340, fluocinolone acetonide, and VEGF-Trap. Pegaptanib sodium injection is an anti-VEGF inhibitor approved by the FDA for the treatment of wet AMD and sold under the tradename Macugen™.

In another embodiment, the additional agent is a matrix metalloproteinase inhibitor. In another embodiment, the additional agent is a matrix metalloproteinase inhibitor selected from among tissue inhibitors of metalloproteinases, α₂-macroglobulin, a tetracycline, a hydroxamate, a chelator, a synthetic MMP fragment, a succinyl mercaptopurine, a phosphonamidate, and a hydroxaminic acid.

In one embodiment, the additional agent is 13-cis-retinoic acid.

In one embodiment, the additional agent has the structure of Formula (A):

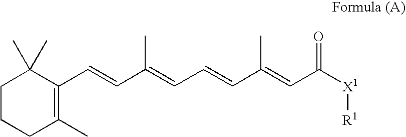

wherein

X₁ is selected from among NR², O, S, CHR²;

R¹ is (CHR²)ₓ-L¹-R³, wherein
x is 0, 1, 2, or 3; L¹ is a single bond or —C(O)—;
R² is a moiety selected from among H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and
R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle.

In another embodiment, the compounds of Formula (A) are with a proviso that R³ is not H when both x is 0 and L¹ is a single bond; or an active metabolite, or a pharmaceutically acceptable prodrug or solvate thereof. In another embodiment, X¹ is NR², wherein R² is H or (C₁-C₄)alkyl. In another embodiment, x is 0. In a further embodiment, x is 1 and L¹ is —C(O)—. In another embodiment, R³ is an optionally substituted aryl. In yet another embodiment, R³ is an optionally substituted heteroaryl. In a further embodiment, X¹ is NH and R³ is an optionally substituted aryl. In a further embodiment, the aryl group has one substituent. In yet a further embodiment, the substituent is a moiety selected from among halogen, OH, O(C₁-C₄)alkyl, NH(C₁-C₄)alkyl, O(C₁-C₄)fluoroalkyl, and N[(C₁-C₄)alkyl]₂. In a further embodiment, the substituent is OH. In another embodiment, the aryl is a phenyl.

In one embodiment, the additional agent is

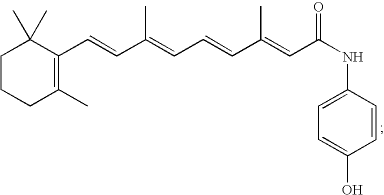

or an active metabolite, or a pharmaceutically acceptable prodrug or solvate thereof.

In another embodiment, the additional agent is 4-hydroxyphenylretinamide; 4-methoxyphenylretinamide; or a metabolite, or a pharmaceutically acceptable prodrug or solvate thereof.

In a further embodiment, the two or more agents are admistered together. In further embodiments, the two or more agents are admistered separately. In some embodiments, the two or more agents are administered in the same pharmaceutical composition. In some embodiments, the two or more agents are administered in separate pharmaceutical compositions. In some embodiments, the methods described herein include prior administration of the additional agent. In some embodiments, the methods described herein include subsequent administration of the additional agent. In some embodiments, the methods described herein include both prior and subsequent administration of the additional agent.

In another embodiment, the method further includes administering to the mammal a therapy selected from among extracorporeal rheopheresis, limited retinal translocation, photodynamic therapy, drusen lasering, macular hole surgery, macular translocation surgery, Phi-Motion, Proton Beam Therapy, Retinal Detachment and Vitreous Surgery, Scleral Buckle, Submacular Surgery, Transpupillary Thermotherapy, Photosystem I therapy, MicroCurrent Stimulation, RNA interference, administration of eye medications such as phospholine iodide or echothiophate or carbonic anhydrase inhibitors, microchip implantation, stem cell therapy, gene replacement therapy, ribozyme gene therapy, photoreceptor/retinal cells transplantation, laser photocoagulation, and acupuncture.

In one embodiment, the method further includes monitoring formation of drusen in the eye of the mammal. In a further embodiment, the method further includes measuring levels of lipofuscin in the eye of the mammal by autofluorescence. In a further embodiment, the method further includes measuring visual acuity in the eye of the mammal. In another embodiment, the method includes conducting a visual field examination on the eye of the mammal. In one embodiment, the visual field examination is a visual field exam.

In another embodiment, the method further includes measuring the autofluorescence of N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, and/or N-retinylidene-phosphatidylethanolamine in the eye of the mammal.

In one embodiment, the ophthalmic condition is macular degeneration. In another embodiment, the macular degeneration is juvenile macular degeneration. In another embodiment, the juvenile macular degeneration is Stargardt Disease. In another embodiment, the macular degeneration is dry form age-related macular degeneration. In yet another embodiment, the macular degeneration is cone-rod dystrophy.

In one embodiment, the ophthalmic condition is a drug-related or drug-induced retinopathy.

In one embodiment, the human is a carrier of the mutant ABCA4 allele for Stargardt Disease or has a mutant ELOV4 gene.

In another embodiment, the method includes determining whether the mammal is a carrier of the mutant ABCA4 allele or has a mutant ELOV4 allele for Stargardt Disease.

In one embodiment, the administration of the agent protects the eye of the mammal from light-induced damage.

In another embodiment, the ophthalmic condition is the spread of geographic atrophy and/or photoreceptor degeneration.

In another embodiment, the method described herein includes an additional treatment for retinal degeneration.

In another embodiment, the human has an ophthalmic condition or trait selected from among Stargardt Disease, recessive retinitis pigmentosa, recessive cone-rod dystrophy, dry-form age-related macular degeneration, exudative age-related macular degeneration, cone-rod dystrophy, retinitis pigmentosa, a lipofuscin-based retinal degeneration, photoreceptor degeneration, and geographic atrophy.

In one embodiment, the method described herein further includes measuring the reading speed and/or reading acuity of the mammal. In another embodiment, the method described herein further includes measuring the number and/or size of the scotoma in the eye of the mammal. In yet another embodiment, the method described herein further includes measuring the size and/or number of the geographic atrophy lesions in the eye of the mammal.

In one embodiment, the activity of a member of the LDL receptor gene family in retina and/or retinol pigment epithileum cells in the eye is the removal of lipofuscin from the retinal pigement epithileum In another embodiment, the activity of Megalin is the removal of lipofuscin from the retinal pigment epithelium. In a further embodiment, the agent increases the removal of lipofuscin from the retinal pigment epithelium.

In another embodiment, described herein are pharmaceutical compositions that include an effective amount of an agent that modulates the activity of a member of the LDL receptor gene family in the retinal pigment epithelium cells in an eye of a mammal; and a pharmaceutically acceptable carrier. In another embodiment, described herein are pharmaceutical compositions that include an effective amount of an agent that modulates the activity of Megalin in the retinal pigment epithelium cells in an eye of a mammal; and a pharmaceutically acceptable carrier. In further embodiments, the pharmaceutical compositions include a pharmaceutically acceptable carrier that is suitable for ophthalmic administration.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description.

All references cited herein, including patents, patent applications, and publications, are hereby incorporated by reference in their entirety.

### BRIEF DESCRIPTION OF THE FIGURES

FIG. 1. Illustrates representative members of the LDL receptor gene family.

FIG. 2. Illustrates detection of the LDL receptor, megalin, in human and rat ocular tissues. (A) An antibody raised against purified megalin (from rat kidney) was used to detect megalin-immunoreactive proteins in extracts prepared from rat and human tissues. A protein extract prepared from rat kidney was used as a positive control for this study. Immunoblot analysis shows the appropriate molecular size band in rat kidney (Rat K, 6 μg, lane 1) and corresponding bands of identical molecular size in rat retina (Ret, 60 μg, lane 2), rat RPE (46 μg, lane 3) and human RPE (20 μg, lane 4). Thyroglobulin, which exists in both dimer (Mr ˜670 kDa) and monomer (Mr ˜335 kDa) forms was used as a size standard. (B) Determination of relative expression of megalin in rat RPE and retina by RT-PCR analysis. Two separate preparations of rat RPE and retina were analyzed in order to obviate the possibility of tissue contamination. Within each preparation, 4 samples were analyzed (1-2 μg of total RNA were used per sample). The data showed that expression of megalin in RPE is ˜15-times higher than that in retina.

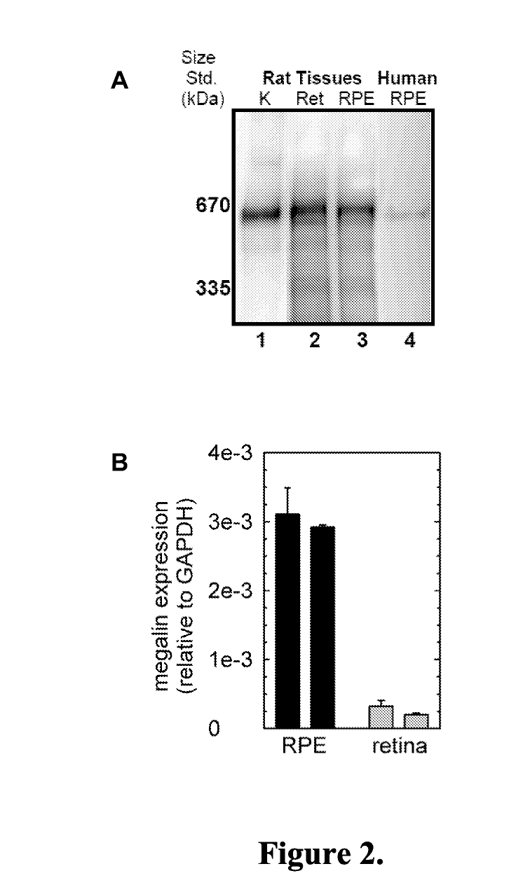

FIG. 3 Illustrates the reduction in molecular size of ocular megalin upon treatment by N-glycosidase F (PNGase F). Megalin is known to be heavily glycosylated. Treatment of megalin with PNGase F has been shown to cause a reduction in protein size as the associated glycans are removed from the protein. Samples of rat eye cup tissue were treated with PNGase F (indicated by “+” in panel A). Control samples were left untreated (indicated by “−” in panel A) The samples were probed with anti-megalin IgG. Rat kidney tissue was used as control. The data show a reduction in molecular size of megalin following PNGase F treatment. The bands from both treated and untreated samples were subjected to limited proteolysis followed by peptide sequencing. The peptide profiles of the two samples were identical (Panel B). MS/MS analysis of one of the peptides revealed a sequence which is unique to megalin (Panel C).

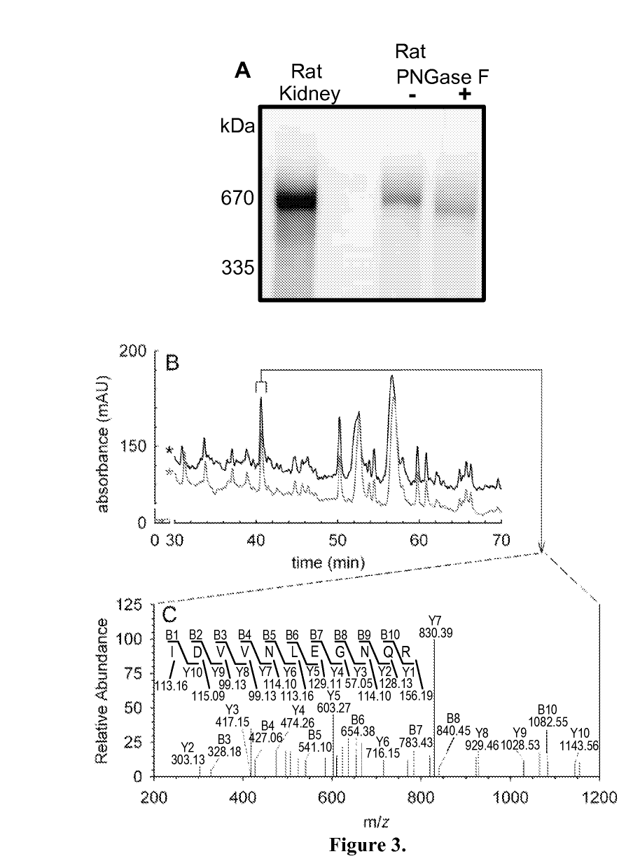

FIG. 4. Illustrates peptide sequencing of the megalin-immunoreactive protein in rat RPE. The megalin immunoreactive proteins identified in FIG. 3 were excised from an acrylamide gel and subject to limited proteolysis by treatment with trypsin. The resulting peptides are separated by liquid chromatography and analyzed by collision-induced dissociation on an electrospray mass spectrometer. The sequenced peptides identified the protein as a low density lipoprotein receptor-related protein 2, also known as megalin (accession: NM 030827.1, GI: 13562118).

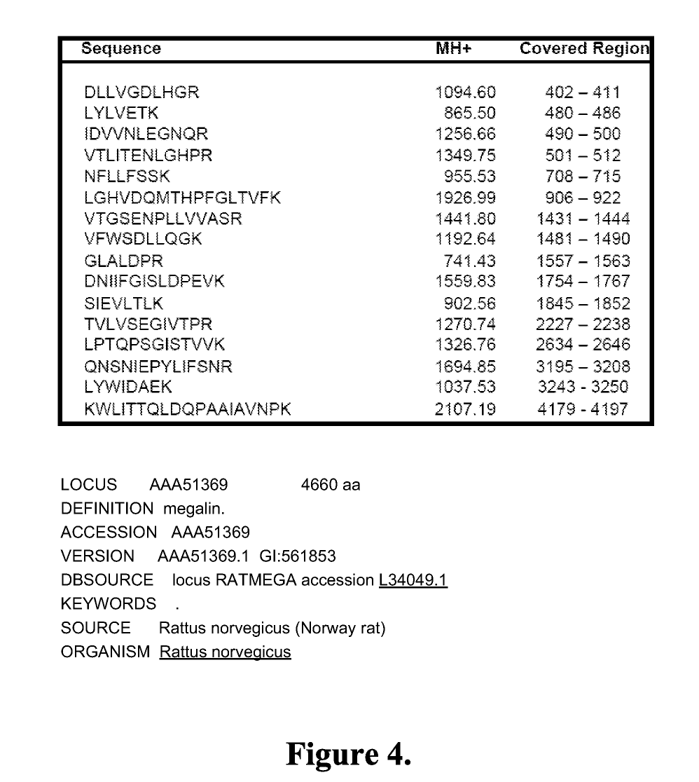

FIG. 5. Illustrates the human RPE cell culture system used to determine the cytolocalization of megalin and in receptor-blocking experiments to determine the role of megalin and other lipoprotein receptors in uptake of holo-retinoid binding proteins. A diagrammatic representation of the apparatus used to culture human RPE cells is shown in panel A. RPE cells are seeded onto a permeable, laminin-containing membrane which is located at the base of a cylindrical vessel. An opening at the top of this vessel permits access to the upper surface of the RPE cell monolayer through the apical media. This unit is placed into a larger cylindrical vessel which provides access the lower surface of the RPE through the basal media. Analysis by electron microscopy reveals that RPE cells cultured in this manner demonstrate proper polarization of apical processes into the apical chamber (panel B).

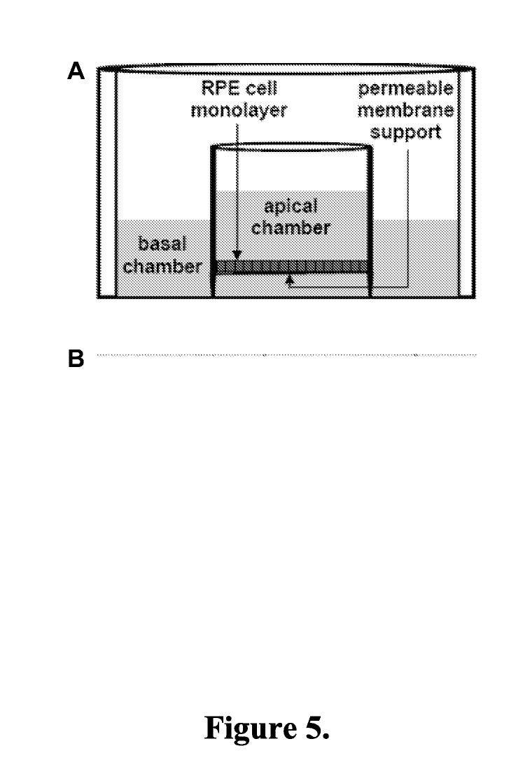

FIG. 6. Illustrates cytolocalization of megalin in human RPE. En face confocal images of megalin immunoreactivity in cultures of human RPE are shown. Megalin immunoreactivity appears as green fluorescence. The panels show serial 1 μm sections starting at the apical RPE cell surface (upper left) and ending at the basal surface (lower right). The staining pattern indicates an apical-lateral localization of megalin. A reconstruction of the Z-axis distribution confirms predominantly apical-lateral localization (bottom panel). Very little megalin is observed at the basal surface.

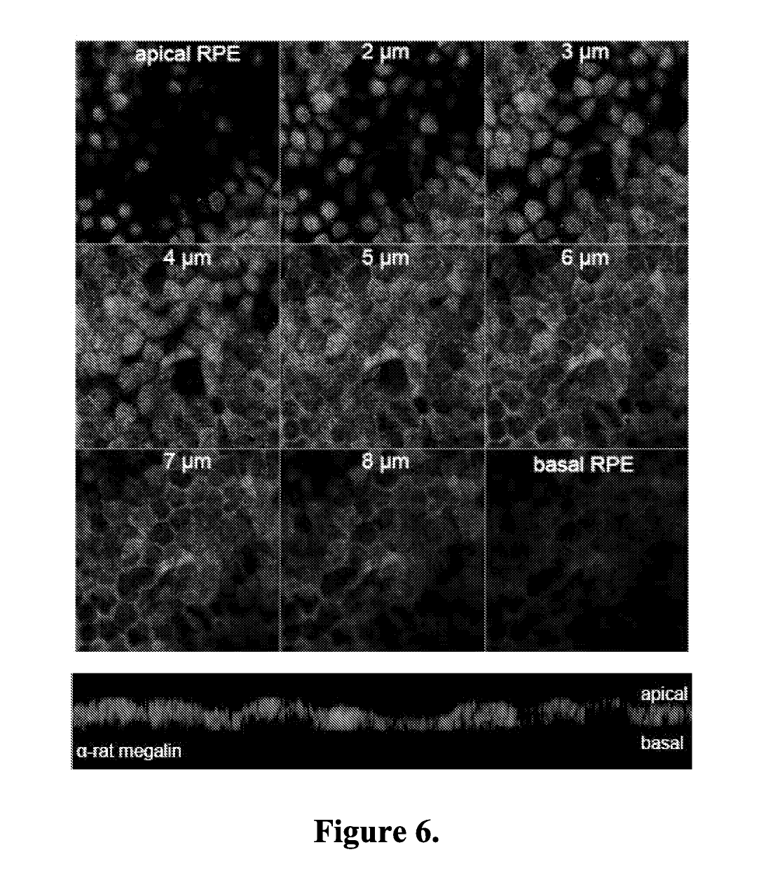

FIG. 7. Illustrates megalin-mediated uptake of RBP-retinol in human RPE. RPE cells uptake RBP-retinol basally from the blood circulation. In addition, RPE cells are also known to synthesize RBP and secrete it across the apical pole of the cell. Antibody blocking experiments were performed to determine whether the megalin plays a role in these processes. A megalin-specific antibody was added to the apical chamber of RPE cell cultures. Control samples received an equivalent concentration of pre-immune rabbit IgG. Following the antibody treatment period (2 hrs at 4° C.), RBP-retinol (10 μM) was added to either the apical (panels A and B) or basal (panels C and D) media. The extent of RBP-retinol uptake was assessed by HPLC quantification of intracellular all-trans retinyl esters (atRE) and all-trans retinol (atROL). UV-vis spectroscopy of the eluted peaks confirmed the identification of atRE and atROL (insets, panel A). The uptake of RBP-retinol from the apical media was ˜3.5-fold higher than uptake from basal media (compare black and red bars in panel E). Pre-treatment of RPE cells with the megalin-specific antibody inhibited both apical (panel B) and basal (panel D) RBP-retinol uptake (40% and 60% inhibition, respectively, panel E). These data implicate megalin in the uptake of RBP-retinol in RPE cells.

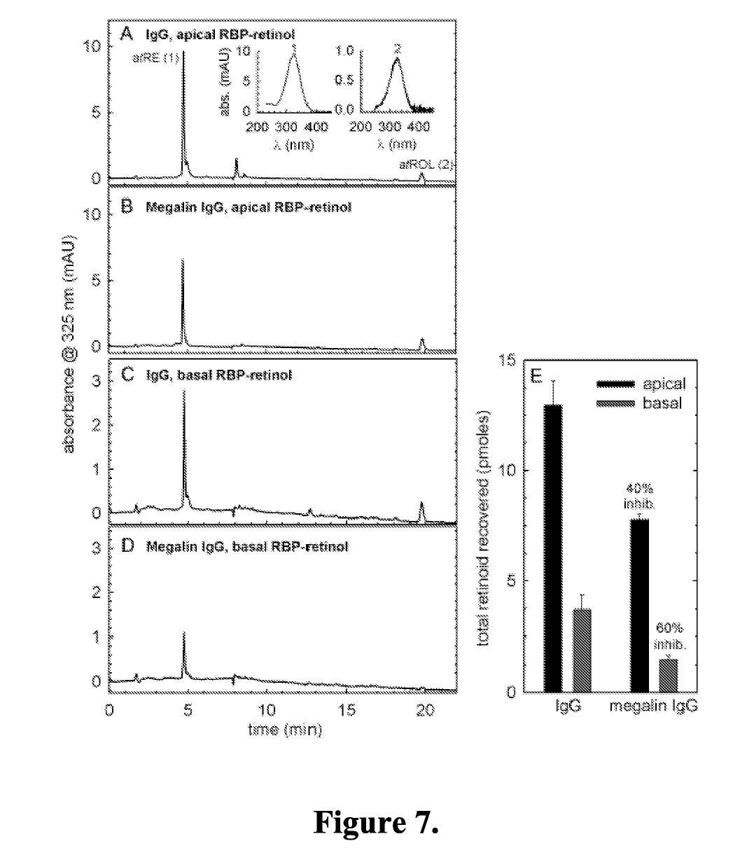

FIG. 8. Illustrates uptake of interphotoreceptor retinoid binding protein (IRBP) by low density lipoprotein receptor-related protein (LRP) and megalin. An antibody specific for the heavy chain of LRP (Mr ˜585 kDa) was used to probe for expression in human RPE. Immunocytochemical studies showed LRP expression predominantly at the apical surfaces of RPE cells (panel A). Immunoblot analyses demonstrated that the LRP and megalin antibodies do not cross-react with one another (panel B). The apical localization of megalin and LRP (see FIGS. 6 and 8, respectively), provided the impetus to determine whether these proteins may play a role in uptake of IRBP-retinol. RPE cell cultures were pre-treated with either pre-immune rabbit IgG (panel C), megalin IgG (panel D), or LRP IgG (panel E) for 2 hrs at 4° C. prior to the apical application of IRBP-retinol (10 μM). The extent of IRBP-retinol uptake was assessed by HPLC quantification of intracellular all-trans retinyl esters (atRE), which was confirmed by uv-vis spectroscopy (inset, panel C). The data reveal significant inhibition of IRBP-retinol by both megalin and LRP IgG (30% and 40% inhibition, respectively, panel F).

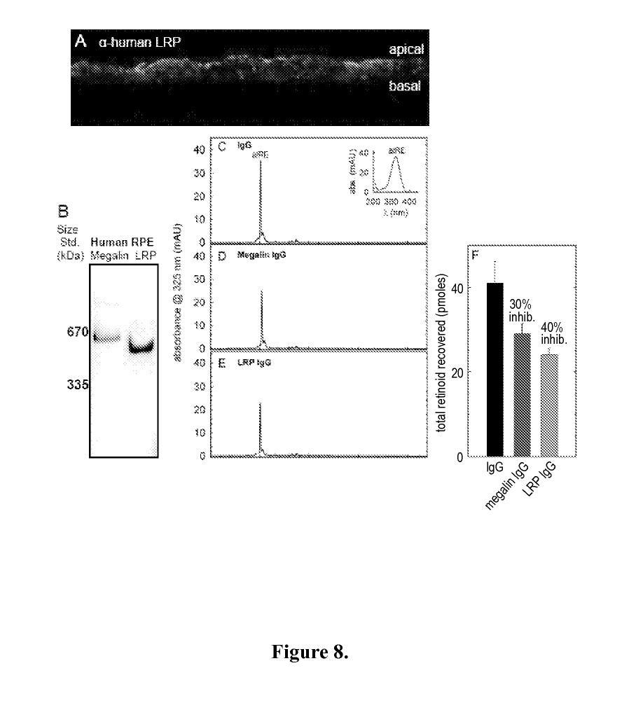

FIG. 9. Illustrates cytolocalization of receptor-associated protein (RAP) in human RPE. RAP is a ˜39-kDa endoplasmic reticulum (ER)-resident protein that functions as a molecular chaperone for several members of the LDL receptor family, including megalin. An antibody raised against human RAP was used to probe for expression of other LDL receptors in cultures of human RPE. Serial sections from the apical RPE cell surface (upper left) toward the basal surface (lower right) show RAP immunoreactivity (green fluorescence) on all surfaces of the RPE. A cross section through the RPE cell monolayer shows intense RAP-immunoreactivity on RPE plasma membranes. RAP is localized in the ER (note immunoreactivity within RPE cells); therefore, the finding of basal RAP-immunoreactivity indicates the presence of RAP-associated LDL receptors on the basal RPE surface.

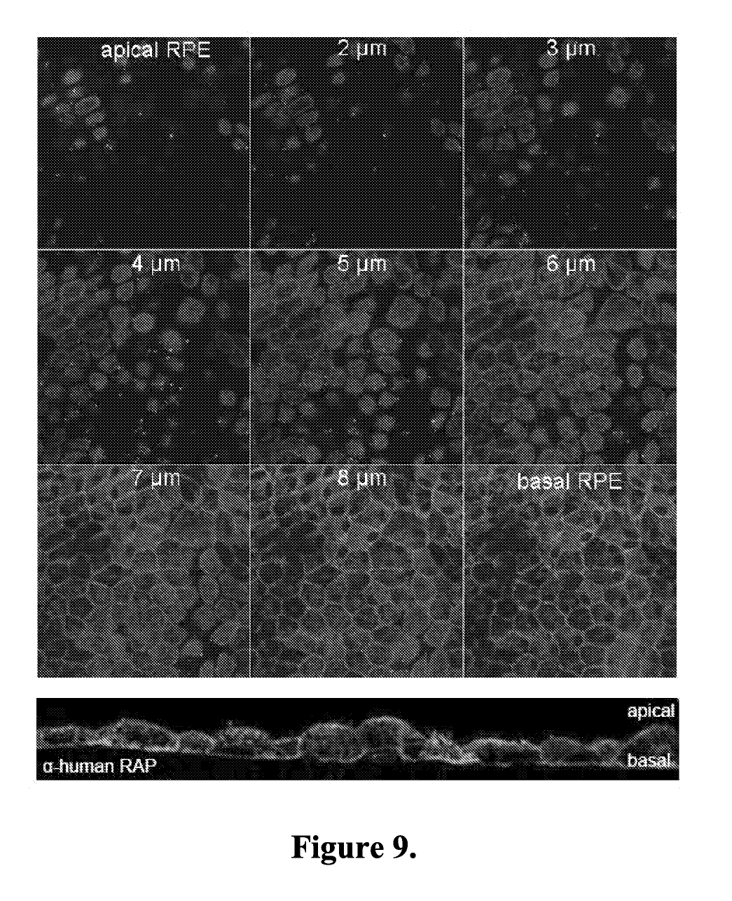

FIG. 10. Illustrates identification and peptide sequencing of a novel low-density lipoprotein receptor-related protein in RPE. An antibody raised against human RAP, which also demonstrates cross-reactivity with megalin, was used to probe for expression of additional LDL receptors in rat RPE. Immunoblots revealed two proteins in rat RPE (panel A, lane 2). The higher molecular weight protein was consistent with megalin (compare to megalin in rat kidney, lane 1). The lower molecular weight protein (red asterisk in panel A) was used for peptide sequencing in order to obtain its identity. Full scan mass spectroscopy detected a peptide (MH +=1650) which was isolated in the mass spectrometer for fragmentation (panel B). Y- and B-ion series generated from this peptide produced a sequence which is highly conserved across LDL family members (FWTD, panel C). The YWTD and FWTD motifs are found as multiple tandem repeats in LDL receptors and have been predicted to form the beta-propeller structure of these proteins. A topological diagram of megalin is provided as an example (panel D). The results show that screening of the entire MS/MS spectra from the digest against a protein database identified the excised protein as a low density lipoprotein receptor-related protein 2 isoform with a mass of 370 kDa (accession: XM 130308.3). Immunocytochemical analysis using the antibody raised against human RAP was used to determine the cytolocalization of the megalin isoform in human RPE. The data show a predominantly basolateral localization (indicated by arrows in panel E). Inverted triangles in panel E indicate megalin expression at the apical pole of the RPE.

FIG. 11. Illustrates inhibition of basal uptake of RBP-retinol in human RPE by RAP. Uptake of RBP-retinol from the circulation occurs at basal surfaces of the RPE. Cytolocalization studies, which revealed RAP-associated LDL receptors on basal RPE plasma membranes, provided the impetus to determine whether these receptors may play a role in basal uptake of RBP-retinol. RAP acts as a chaperone for LDL receptors by binding to the ligand binding domains present on these receptors. Thus, RAP can also be utilized as a ligand binding antagonist. RAP was added to the basal chamber of RPE cell cultures. Control samples received an equivalent concentration of pre-immune rabbit IgG. Following the antibody treatment period (2 hours at 4° C.), RBP-retinol (10 μM) was added to the basal media. The extent of RBP-retinol uptake was assessed by HPLC quantification of intracellular all-trans retinyl esters (atRE) and (atROL). UV-vis spectroscopy of the eluted peaks confirmed the identity of atRE and atROL (insets, panel A). RPE cells which were treated with pre-immune IgG showed robust uptake and esterification of atROL (panel A). In contrast, RPE cells pre-treated with RAP (panel B) demonstrated a significantly reduced uptake of RBP-retinol. Quantitation of the data reveal a 47% inhibition of RBP-retinol uptake by RAP (panel C).

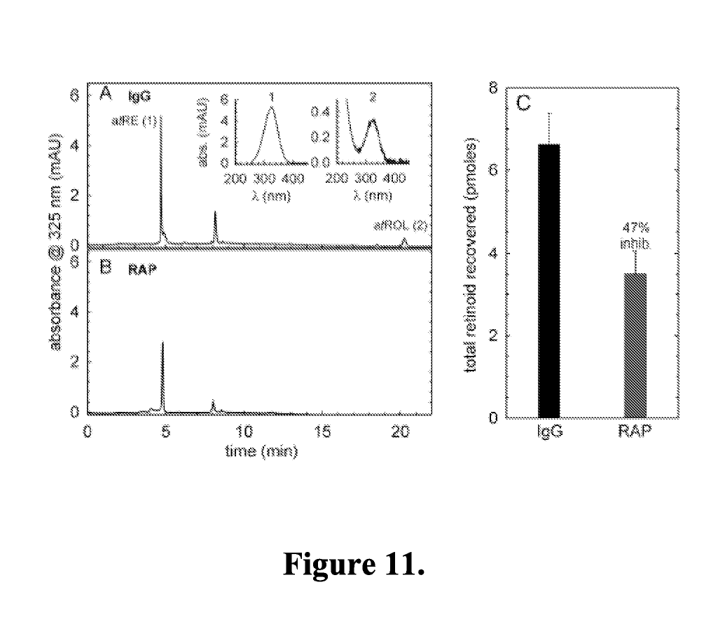

FIG. 12. Illustrates megalin protein level in the eye cup from mice with different serum RBP-retinol levels. LDL receptors function to uptake RBP-retinol into RPE. RBP knockout mouse and MPR-treated mouse have lower serum RBP-retinol level. Expression of megalin in eyecup tissues from these mice were examined by immunoblot. Membrane fractions of mouse eyecups were prepared from wild-type mouse (WT), RBP knockout mouse (RBP−/−), ABCR null (abcr−/−) and MPR-treated mice (MPR). Two immunoreactive bands were detected (indicated by white arrows in the figure). The data show reduced expression of both proteins in RBP knockout mice and MPR-treated mice compared to age-matched wild-type and abcr knockout mice.

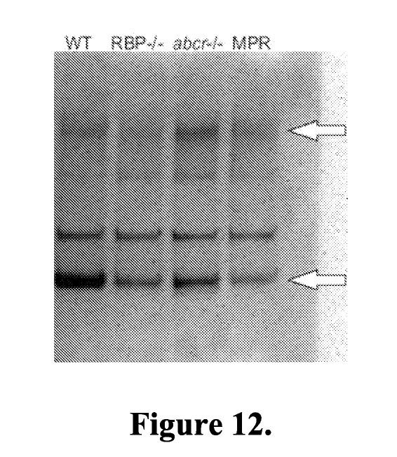

FIG. 13. Illustrates uptake of RBP- and IRBP-retinol into human RPE. Holo-RBP and IRBP were covalently labeled with a fluorescent probe (Alexa Fluor 488). The labeled proteins (RBP* and IRBP*) were added to either the basal (RBP*) or apical (IRBP*) compartments of the RPE cell culture system. Following a 1 hour incubation at 37° C., the media were removed, the cells were extensively washed and the tissues samples analyzed by fluorescence microscopy. The data show pronounced uptake of both RBP* and IRBP* into RPE cells indicating the presence of an endocytic mechanism.

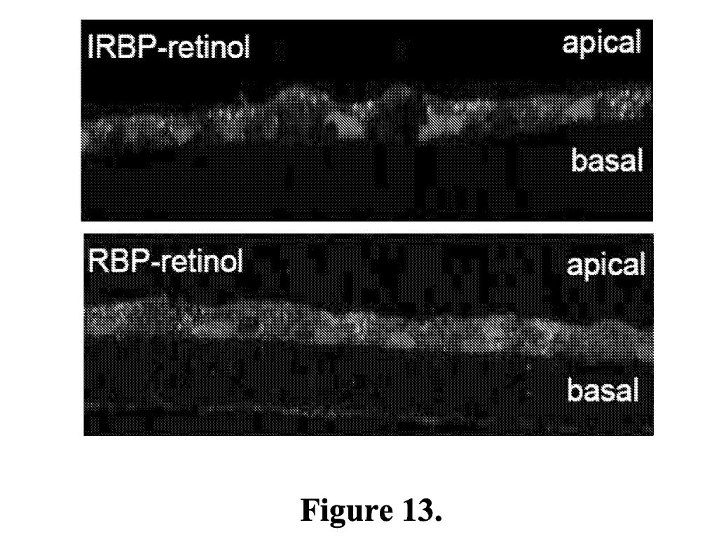

FIG. 14. Illustrates that RAP inhibits basal uptake of RBP-retinol and apical uptake of IRBP-retinol. Uptake of IRBP* and RBP* by RPE cells was monitored before (panels A and B, respectively) and after (panels D and E, respectively) treatment with the LDL receptor antagonist, RAP. RAP treatment completely suppressed basal uptake of RBP* and apical uptake of IRBP*. These data indicate that the uptake process is mediated by LDL receptors. To ensure that retinol was also taken into the RPE cells, the cells were washed, collected and analyzed for retinoid content by HPLC. Uptake of retinol into RPE cells results in rapid esterification resulting in retinyl ester formation. Quantitation of retinyl esters showed that the RPE cells do indeed internalize retinol and esterify it to retinyl esters (panel C). RAP pre-treatment caused a ˜50% reduction in retinol uptake as measured by retinyl ester content (panel F).

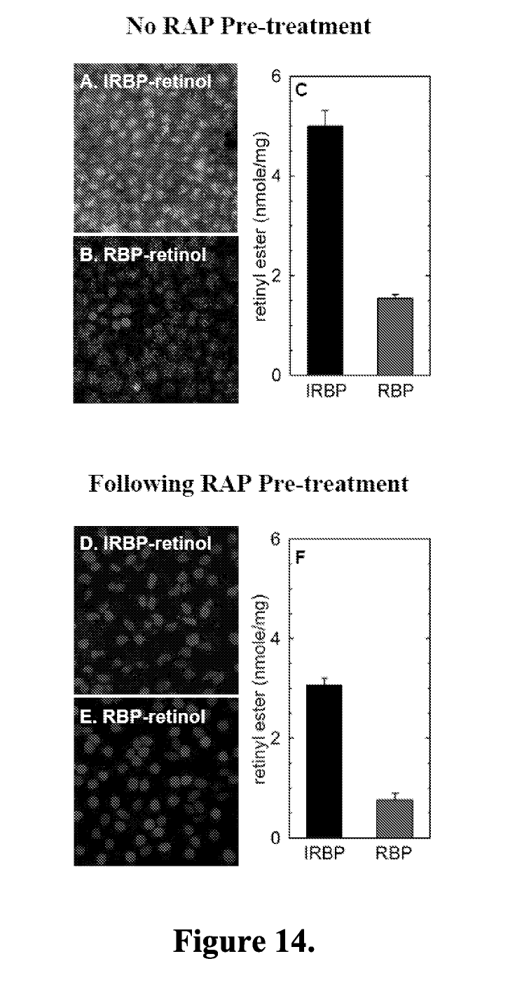

FIG. 15. Illustrates that transfer of retinol to CRBP from IRBP-retinol proceeds at a greater rate than from RBP-retinol. The higher rate of retinol uptake from IRBP-retinol compared to RBP-retinol (see FIG. 14, panel C) suggested that retinol transfer from IRBP-retinol to the intracellular retinol acceptor, cellular retinol binding protein (CRBP), may proceed greater rate. To address this possibility, we incubated IRBP-retinol or RBP-retinol with an equimolar concentration of CRBP (10 μM) and monitored spectral shift using authentic CRBP-retinol as a reference spectra. The excitation spectra of CRBP-retinol (dashed lines in panels A and B) is distinct from that of either IRBP-retinol (red trace in panel A) or RBP-retinol (green trace in panel B). Following 1 minute incubation at 37° C., there is an obvious shift in the excitation spectra of IRBP-retinol indicating transfer of retinol from IRBP to CRBP. In contrast, no shift in the excitation spectra of RBP-retinol was observed even after 2 hours of incubation with CRBP.

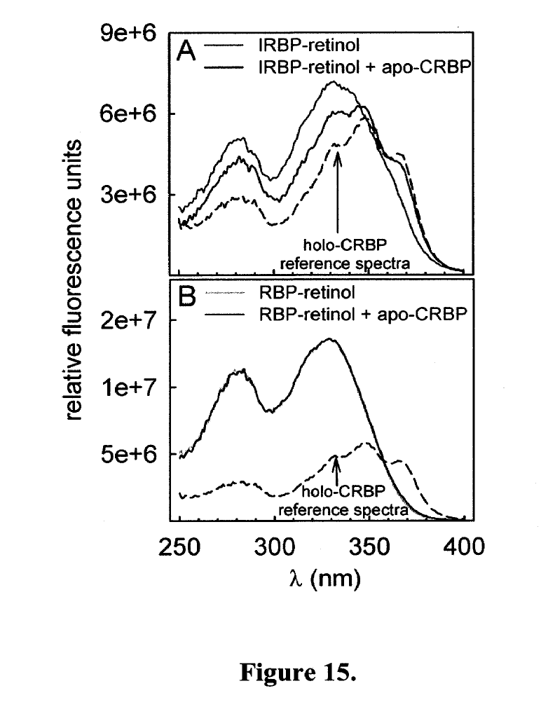

FIG. 16. Illustrates a hypothetical model for uptake of RBP-retinol and IRBP-retinol. Uptake of RBP-retinol from the basal RPE and subsequent association with CRBP requires degradation of the RBP protein through the lysosomal pathway. In contrast, association of retinol to CRBP from IRBP retinol may proceed prior to protein degradation as retinol is transferred from IRBP directly to CRBP.

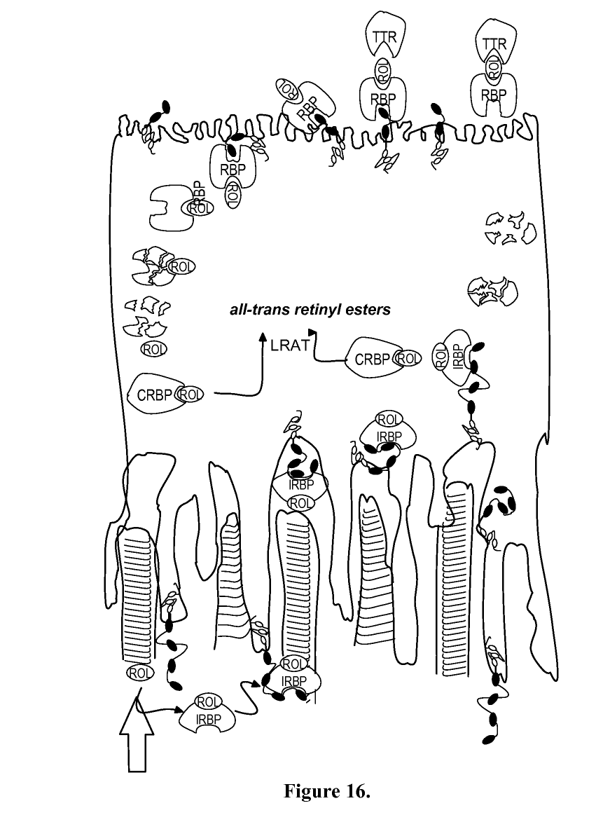

### DETAILED DESCRIPTION OF THE INVENTION

Two fundamental processes of vertebrate vision sustain light perception: transformation of the light signal into chemical changes within photoreceptor cells and a regeneration process involving the retinal pigment epithelial cells (RPE). Isomerization of the visual pigments' chromophore, 11-cis retinal to all-trans retinal, triggers a set of reactions, collectively termed phototransduction. Before light sensitivity can be regained, the resulting all-trans-retinal must dissociate from the opsin apoprotein and isomerize to 11-cis-retinal. The photolyzed product, all-trans retinal, is first reduced to all-trans retinol in the photoreceptors and then converted back to 11-cis retinal in the RPE in an enzymatic process referred to as the visual cycle. (Rando, R.R. The Biochemistry of the Visual Cycle. Chem. Rev. 101, 1881-1896, 2001). The photoreceptors are separated from the apical surface of the RPE by the subretinal space, which contains a specialized extracellular material referred to as the interphotoreceptor matrix (IPM). The IPM mediates key interactions between the photoreceptors and RPE including adhesion, phagocytosis, outer segment stability, nutrient exchange, development, and vitamin A trafficking in the visual cycle.

Vitamin A circulates in the blood and enters the eye in the form of all-trans retinol. This form is taken up from the circulation by the basal membrane of the retinal pigment epithileum (RPE) cells, which enzymatically convert all-trans retinol into all-trans retinyl esters. The RPE contain the enzymatic machinery necessary for the conversion of all-trans retinol esters to 11-cis retinal. The latter retinoid is transported from the RPE to photoreceptor outer segments (POS) in the retina, where it associates with opsin to form rhodopsin.

An important interaction that occurs between the RPE and photoreceptors is the exchange of retinoids in the visual cycle. Interphotoreceptor retinoid-binding protein (IRBP), a photoreceptor secretory glycoprotein, participates in the visual cycle by solubilizing retinoids within the IPM, by targeting the delivery of all-trans retinol to the RPE, by promoting the release of 11-cis retinal from the RPE, and by targeting its delivery to the outer segments. IRBP is a glycoprotein with a molecular weight of approximately 140 kDa. The amino acid sequence and cDNA are known. Trafficking of retinoids between the RPE and IPM is mediated by receptor mediated transcytosis. IRBP and/or IRBP-retinol complex and/or IRBP-retinal complex binds to receptor proteins, such as, for example, members of the LDL receptor gene family, on the apical membrane of RPE cells. In some embodiments, the members of the LDL receptor gene family that bind IRBP and/or IRBP-retinol complex and/or IRBP-retinal complex are, for example, megalin or megalin-related proteins.

The RPE forms part of the retinal-blood barrier and also supports the function of photoreceptor cells. The RPE cell layer acts as a support for photoreceptors performing such functions as nutrient and waste transport, as well as phagocytosis of shed POS and degradation/processing of the phagocytozed POS within the (acidic) lysosomal apparatus of the RPE cells. However, this processing becomes perturbed by the prooxidant environment of the retina and is responsible for the intralysosomal formation and accumulation of lipofuscin, a complex polymer of peroxidized lipids and protein residues. Oxidative events in the RPE have been linked to such disease states as age-related macular degeneration (AMD).

The onset of AMD has been correlated with the accumulation of complex and toxic biochemicals (toxins) in and around the RPE and lipofuscin in the RPE. The accumulation of these retinotoxic compounds in the eye is one of the most important known risk factors in the etiology of AMD. In at least some forms of macular degeneration, accumulation of lipofuscin in the RPE is due in part to the phagocytosis of spent outer segments of rod cells. Retinotoxic compounds form in the discs of rod photoreceptor outer segments. The retinotoxic compounds in the disc are brought into the RPE, where they impair further phagocytosis of POS and cause apoptosis of the RPE. Photoreceptors cells, including cone cells essential for daytime vision, lose RPE support and die.

One of the retinotoxic compounds formed in the discs of rod outer segments is N-retinylidene-N-retinylethanolamine (A2E), which is an important component of the retinotoxic lipofuscins. A2E is normally formed in the discs but in such small amounts that it does not impair RPE function upon phagocytosis. However, in certain pathological conditions, so much A2E can accumulate in the disc that the RPE is “poisoned” when the outer segment is phagocytosed. A2E has been shown to impair lysosomal degradation functions of RPE cells in vitro by elevating the intralysosomal pH.

A2E is produced from all-trans-retinal, one of the intermediates of the rod cell visual cycle. During the normal visual cycle, all-trans-retinal is produced inside rod outer-segment discs. The all-trans-retinal can react with phosphatidylethanolamine (PE), a component of the disc membrane, to form N-retinylidene-PE. Rim protein (RmP), an ATP-binding cassette transporter located in the membranes of rod outer-segment discs, then transports all-trans-retinal and/or N-retinylidene-PE out of the disc and into rod outer-segment cytoplasm. The environment there favors hydrolysis of the N-retinylidene-PE. The all-trans-retinal is reduced to all-trans-retinol in the rod cytoplasm. The all-trans-retinol then crosses the rod outer-segment plasma membrane into the extracellular space and is taken up by cells of the RPE. The all-trans-retinol is converted through a series of reactions to 11-cis-retinal, which returns to the photoreceptor and continues in the visual cycle.

Defects in RmP can disrupt the visual cycle by impeding removal of all-trans-retinal from the disc. In a recessive form of macular degeneration called Stargardt's disease, the gene encoding RmP, abcr, is mutated, and the transporter is nonfunctional. As a result, all-trans-retinal and/or N-retinylidene-PE become trapped in the disc. The N-retinylidene-PE can then react with another molecule of all-trans-retinal to form A2E. As noted above, some A2E is formed even under normal conditions; however, its production is greatly increased when its precursors accumulate inside the discs due to the defective transporter, and can thereby cause macular degeneration.

Other forms of macular degeneration may also result from pathologies that result in lipofuscin accumulation. A dominant form of Stargardt's disease, known as chromosome 6-linked autosomal dominant macular dystrophy, is caused by a mutation in the gene encoding elongation of very long chain fatty acids-4, ELOV4.

The highly organized membranous discs of the photoreceptor outer segments require lipoproteins, cholesterol and phospholipids for their formation. The RPE may be involved in the homeostasis of these lipids, lipoproteins and cholesterol in the retina. The RPE possess receptor proteins, such as memebers of the LDL receptor gene family, that uptake lipoproteins and lipids, as well as damaged/peroxidized lipoproteins and lipids, such as that which accumulates in RPE and aortic endothelium during vitamin E deficiency or in macrophages during atherogenesis (Hayes et al. Retinal Pigment Epithelium Possesses Both LDL and Scavenger Receptor Activity. IOVS, vol. 30, no. 2, 225-232, 1989). The uptake of peroxidized lipoproteins may accentuate and/or accelerate the disruption of normal RPE functioning and contribute to the pathogenesis of AMD. Oxidized low densisity lipoprotein has been shown to inhibit photoreceptor outer segment phagocytosis in RPE cells (Gordiyenko et al. RPE cells Internalize Low-Density Lipoprotein (LDL) and Oxidixed LDL (oxLDL) in Large Quantities in vitro and in vivo. IOVS, vol. 45, no. 8, 2822-2829, 2004). The RPE is capable of internalizing LDL and accumulating LDL deposits in vivo. It also has been shown that plasma LDL can get into the RPE very efficiently while carrying other molecules, such as, for example, vitamin E as well as oxidized LDL. LDL also has been shown to be a transport vehicle for A2E into lysosomes of the RPE (Schutt et al. IOVS, vol. 41, no. 8, 2303-2308, 2000). The internalization of oxidized lipoproteins also may occur through recognition and binding of the oxidized phospholipids on the surface of the oxidized lipoprotein molecule by receptor proteins, such as, for example, members of the LDL receptor gene family. In one embodiment, are methods and compositions for treating an ophthalmic condition in an eye of a mammal that includes administering to the mammal an effective amount of an agent that modulates the activity of a member of the LDL receptor gene family in the retina and/or retinal pigment epithelium cells in the eye of the mammal, wherein the activity of the member of the LDL receptor gene family is the uptake of lipoproteins and/or oxidized lipoproteins.

Further information regarding the anatomical organization of the vertebrate eye, the visual cycle for regeneration of rhodopsin, and the biogenesis of A2E-oxiranes is provided in U.S. patent application Ser. No. 11/150,641, filed Jun. 10, 2005, PCT Pat. App. No. US 2005/29455, filed Aug. 17, 2005; U.S. patent application Ser. No. 11/258,504, filed Oct. 25, 2005; U.S. patent application Ser. No. 11/296,909, filed Dec. 8, 2005; and U.S. patent application Ser. No. 11/267,395, filed Nov. 4, 2005; the contents of which are incorporated by reference in their entirety.

### Macular or Retinal Degenerations and Dystrophies

Macular degeneration (also referred to as retinal degeneration) is a disease of the eye that involves deterioration of the macula, the central portion of the retina. Approximately 85% to 90% of the cases of macular degeneration are the “dry” (atrophic or non-neovascular) type. In dry macular degeneration, the deterioration of the retina is associated with the formation of small yellow deposits, known as drusen, under the macula; in addition, the accumulation of lipofuscin in the RPE leads to photoreceptor degeneration and geographic atrophy. This phenomena leads to a thinning and drying out of the macula. Administration of at least one agent that modulates the activity of a member of the LDL receptor gene family in retina and/or RPE cells, such as for example, a megalin-modulating agent, to a mammal can reduce the formation of, or limit the spread of, photoreceptor degeneration and/or geographic atrophy in the eye of the mammal.

In “wet” macular degeneration new blood vessels form (i.e., neovascularization) to improve the blood supply to retinal tissue, specifically beneath the macula, a portion of the retina that is responsible for our sharp central vision. The new vessels are easily damaged and sometimes rupture, causing bleeding and injury to the surrounding tissue. Although wet macular degeneration only occurs in about 10 percent of all macular degeneration cases, it accounts for approximately 90% of macular degeneration-related blindness. Growth promoting proteins called vascular endothelial growth factor, or VEGF, have been targeted for triggering this abnormal vessel growth in the eye. This discovery has lead to aggressive research of experimental drugs that inhibit or block VEGF. Studies have shown that anti-VEGF agents can be used to block and prevent abnormal blood vessel growth. Such anti-VEGF agents stop or inhibit VEGF stimulation, so there is less growth of blood vessels. Such anti-VEGF agents may also be successful in anti-angiogenesis or blocking VEGF's ability to induce blood vessel growth beneath the retina, as well as blood vessel leakiness. Administration of at least one agent that modulates the activity of a member of the LDL receptor gene family in retina and/or RPE cells, such as for example, a megalin-modulating agent, to a mammal can reduce the formation of, or limit the spread of, wet-form age-related macular degeneration in the eye of the mammal. Similarly, an agent that modulates the activity of a member of the LDL receptor gene family in retina and/or RPE cells, such as, for example, a megalin-modulating agent, can be used to treat choroidal neovascularization and the formation of abnormal blood vessels beneath the macula of the eye of a mammal.

Stargardt Disease is a macular dystrophy that manifests as a recessive form of macular degeneration with an onset during childhood. Mutations in the human ABCA4 gene for Rim Protein (RmP) are responsible for Stargardt Disease. Histologically, Stargardt Disease is associated with deposition of lipofuscin pigment granules in RPE cells. Mutations in ABCA4 have also been implicated in recessive retinitis pigmentosa, recessive cone-rod dystrophy, and non-exudative age-related macular degeneration, although the prevalence of ABCA4 mutations in AMD is still uncertain. Similar to Stargardt Disease, these diseases are associated with delayed rod dark-adaptation. Lipofuscin deposition in RPE cells is also seen prominently in AMD, and some cases of retinitis pigmentosa. In addition, an autosomal dominant form of Stargardt Disease is caused by mutations in the ELOV4 gene.

In addition, there are several types of macular degenerations that affect children, teenagers or adults that are commonly known as early onset or juvenile macular degeneration. Many of these types are hereditary and are looked upon as macular dystrophies instead of degeneration. Some examples of macular dystrophies include: Cone-Rod Dystrophy, Corneal Dystrophy, Fuch's Dystrophy, Sorsby's Macular Dystrophy, Best Disease, and Juvenile Retinoschisis, as well as Stargardt Disease.

### Retinol Absorption in Ocular Tissues

Retinoids (vitamin A and its analogs) are required to maintain many essential physiologic processes, including normal reproduction, normal immunity, normal growth and cellular differentiation, and normal vision. All retinoids present in the body must be acquired from the diet. Following consumption of a vitamin-A rich meal, along with other dietary lipids, dietary retinoids (modified as retinyl esters) are packaged in chylomicrons and stored in hepatic stellate cells.

To meet the body's need for retinoids, the liver secretes into circulation retinol bound to a 21 kDa protein, retinol-binding protein (RBP). Retinol-RBP is found in a 1:1 molar complex with a 55 kDa protein, tranthyretin (TTR) Before the retinol-RBP holoprotein can be delivered to extra-hepatic target tissues, such as the eye, it must bind with transthyretin (TTR). (Zanotti and Bemi, Vitam. Horm., 69:271-95 (2004)). It is this secondary complex that allows retinol to remain in the circulation for prolonged periods. Association with TTR facilitates RBP release from hepatocytes, and prevents glomerular filtration and renal catabolism of RBP.

A mouse strain deficient in transthyretin is viable and fertile, yet exhibits significantly depressed levels of serum retinol, retinol-binding protein, and thyroid hormone, confirming transthyretin's role in maintaining normal levels of these metabolites in circulating plasma (Episkopou et al., Proc Natl Acad Sci USA, 1993, 90, 2375-2379). Furthermore, transthyretin reabsorption by the kidneys is mediated by the lipoprotein receptor megalin (Sousa et al., J Biol Chem, 2000, 275, 38176-38181). This reabsorption serves as a means for preventing hormone loss in urine.

Megalin, also known as gp330, is a member of the LDL receptor gene family and is located in the endocytic pathway in proximal tubule cells. Megalin is a 600 kDa (in its glycosylated form) membrane-bound endocytic protein that acts as a scavenger receptor for the absorption of proteins from tubular fluid (Christensen et al., J. Am. Soc. Nephrol. 10: 2224-2236, 1999). Among the ligands that bind to megalin with high affinity are vitamin carrier protein, such as, for example, retinoid binding proteins, such as, for example, retinol binding protein (RBP) and interphotoreceptor retinoid binding protein (IRBP). Megalin is the most abundant endocytic receptor protein in the endocytic pathway in proximal tubule cells and is responsible for the endocytic uptake of proteins, including RBP, from the glomerular ultrafiltrate.

The retinol-RBP-TTR complex is delivered to target tissues where retinol is taken up and utilized for various cellular processes. Delivery of retinol to cells through the circulation by the RBP-TTR complex is the major pathway through which cells and tissues acquire retinol. Unlike other tissues in the body, the eye takes up postprandial retinol very poorly. The eye must rely on retinol bound to RBP as its primary means for acquiring the retinoid needed for normal visual pigment formation (Vogel et al., Biochemistry, 2002, 41, 15360-15368).

### Retinol Binding Protein (RBP)

Retinol binding protein, or RBP, is a single polypeptide chain, with a molecular weight of approximately 21 kDa. RBP has been cloned and sequenced, and its amino acid sequence determined. Colantuni et al., Nuc. Acids Res., 11:7769-7776 (1983). The three-dimensional structure of RBP reveals a specialized hydrophobic pocket designed to bind and protect the fat-soluble vitamin retinol. In plasma, approximately 95% of the plasma RBP is associated with transthyretin (TTR) in a 1:1 mol/mol ratio, wherein essentially all of the plasma vitamin A is bound to RBP. TTR is a well-characterized plasma protein consisting of four identical subunits with a molecular weight of 54,980. The full three-dimensional structure, elucidated by X-ray diffraction, reveals extensive beta-sheets arranged tetrahedrally. Blake et al., J. Mol. Biol., 121:339-356 (1978). The complexation of TTR to RBP-retinol is thought to reduce the glomerular filtration of retinol, thereby increasing the half-life of retinol and RBP in plasma by about threefold.

Retinol uptake from its complexed retinol-RBP-TTR form into cells, such as retina and RPE cells, occurs by binding of RBP to cellular receptors, such as, for example, members of the LDL receptor gene family, on target cells. In some embodiments, the member of the LDL receptor gene family is megalin or a megalin-related protein. In some embodiments, the member of the LDL receptor gene family is megalin. This interaction leads to endocytosis of the RBP-receptor complex and subsequent release of retinol from the complex, or binding of retinol to cellular retinol binding proteins (CRBP), and subsequent release of apoRBP by the cells into the plasma. Other pathways contemplate alternative mechanisms for the entry of retinol into cells, including uptake of retinol alone into the cell. See Blomhoff (1994) for review.

In the kidneys, RBP has been shown to bind to purified megalin by BIAcore experiments and that the retinod binding protein and retinol is found in the urine of megalin-deficient mice but is absent in control mice (Christensen E I. et al. J. Am. Soc. Nephrol. 10:685-695, 1999). Endogenous RBP was found by immunocytochemistry in the proximal tubules of control mice but was absent in megalin knockout mice. Other tissues, such as, for example, the retina and RPE, also express megalin or megalin-related proteins and are capable of binding RBP and internalizing RBP.

A2E, the major fluorophore of lipofuscin, is formed in macular or retinal degeneration or dystrophy, including age-related macular degeneration and Stargardt Disease, due to excess production of the visual-cycle retinoid, all-trans-retinaldehyde, a precursor of A2E. Reduction of the amount of vitamin A, 11-cis-retinal and all-trans retinal in the retina and RPE, therefore, would be beneficial in reducing A2E and lipofuscin build-up, and treatment of age-related macular degeneration.

Reduction of serum retinol levels is one approach contemplated for the treatment of ocular disorders. Another approach for the treatment of ocular disorder is to modulate the uptake of retinol into ocular tissues. In one approach, the activity of a member of the LDL receptor gene family that is expressed in retina and/or RPE cells is modulated with an agent, such that the retinol, retinol-RBP, and/or retinol-RBP-TTR complex is/are prevented from binding to said receptor(s), thereby inhibiting entry of the retinoid into the RPE and/or retina.

In another approach, the activity of a member of the LDL receptor gene family that is expressed in retina and/or RPE cells is modulated with an agent, such that the retinol, retinol-RBP, retinol-RBP-TTR and/or retinol-IRBP complex is/are prevented from binding to said receptor(s). Inhibition of binding of retinol, retinol-RBP, retinol-RBP-TTR and/or retinol-IRBP complex to a member of the LDL receptor gene family in retina and/or RPE cells may disrupt the visual cycle. Disruption of the visual cycle can decrease the amount or accumulation of toxic chemicals that are present in retina and/or RPE cells in certain ophthalmic conditions.

Identified herein are receptor proteins belonging to the LDL receptor gene family in retina and retina pigment epithelial (RPE) cells. In one embodiment, the receptor protein belonging to the LDL receptor gene family is a retinoid binding protein receptor. In some embodiments, the receptor protein is megalin. In some embodiments, the receptor protein is a megalin-related protein. In some embodiments, the receptor protein is LRP. In some embodiments, the receptor protein is a LRP-related protein. In some embodiments, the receptor protein is STRA6 or a STRA6-related protein. In some embodiments, the receptor protein is STRA6. In some embodiments, the receptor protein is a STRA6-related protein. STRA6 has been identified as a membrane receptor for retinol binding protein and evidence is shown that STRA6 can mediate cellular uptake of vitamin A. Additional information regarding STRA6 can be found in U.S. Pat. No. 7,173,115, Kawaguchi R. et al., 2007, Science 315: 820-25, and Blaner W. 2007, Cell Metabolism 5: 164-66, which are all incorporated by reference in their entirety. Additional information regarding STRA6 related protein can be found in patent applications US 2007/0128188, US 2003/0021788, and US 2002/0156252, which are all incorporated by reference in their entirety.

Provided herein are methods of preventing, treating or curing visual defects by antagonizing, agonizing, and/or modulating the activity of transcytotic receptors in retina and RPE cells, which belong to the LDL receptor gene family. In some cases, a receptor belonging to the LDL receptor gene family on the basal membrane of the RPE is antagonized with an LDL receptor gene family binding agent, thus preventing binding and uptake of RBP-retinol, RBP-retinol-TTR, or retinol into the RPE. In other cases, a receptor belonging to the LDL receptor gene family on the apical membrane of the RPE is antagonized with a LDL receptor gene family binding agent, thus preventing binding and transcytosis of retinol, retinal, IRBP-retinol, IRBP-retinal, or IRBP into or out of RPE cells.

### Toxic Effects of Drugs and Toxins

A variety of ocular disorders or conditions are a result of treatment with drugs and toxins. For example, antibiotics, such as aminoglycosides, are used frequently in opthalmology to treat or prevent bacterial infections. These antibiotics are known to be ototoxic, nephrotoxic as well as retinal toxic. (D'Amico et al. Retinal Toxicity of Intravitreal Gentamicin Invest. Ophthalm. Vis. Sci. 25:564-572, 1984; Campochiaro et al. Arch. Opthalmol. 113(3):262-263, 1995; Grizzard, Arch Opthalmol. 112(1):48-53, 1994). Aminoglycosides such as, for example, arbekacin, gentamicin, kanamycin, neomycin, paramycin, ribostamycin, lividomycin, amikacin, dibekacin, butakacin, tobramycin, streptomycin, dihydrostroptomycin, sisomicin, verdamicin, netilmicin, and butikacin have been shown to accumulate in ocular tissue and/or exert toxic effects in the eye. In one embodiment, an agent prevents binding of an antibiotic to a member of the LDL receptor gene family in retina and/or RPE cells. In another embodiment, an agent provided herein prevents the binding of and transcytosis of an antibiotic by a member of the LDL receptor gene family in retina and/or RPE cells.

Other disorders of the eye are related to drugs that display ocular toxicity. Certain pharmaceutical drugs accumulate in retina and/or RPE cells in the eye. In certain cases, therapuetic drugs are metabolized in ocular tissues, such as, for example, retina and/or RPE cells in the eye. The retina, replete with cytochromes P450 and myeloperoxidase, may serve to activate xenobiotics to oxidants, resulting in ocular injury. These activated agents may directly form retinal adducts or may diminish ocular reduced glutathione concentrations. (Toler, Exp. Biol. Med. 229:607-615, 2004). In one embodiment, inhibition of the binding of therapuetic drugs to members of the LDL receptor gene family in retina and/or RPE cells reduces ocular toxicity related to the use of said therapuetic drugs. In another embodiment, binding and transcytosis of a therapuetic drug by a member of the LDL receptor gene family in retina and RPE cells is inhibited by an agent described herein.

Retinopathies are divided into two broad categories, simple or nonproliferative retinopathies and proliferative retinopathies. The simple retinopathies include the defects identified by bulging of the vessel walls, by bleeding into the eye, by small clumps of dead retinal cells called cotton wool exudates, and by closed vessels. This form of retinopathy is considered mild. The proliferative, or severe, forms of retinopathies include the defects identified by newly grown blood vessels, by scar tissue formed within the eye, by closed-off blood vessels that are badly damaged, and by the retina breaking away from the mesh of blood vessels that nourish it (retinal detachment).

A variety of therapeutic drug-induced retinal effects have been observed in the course of medical treatrnent. (LeBlanc et al. Regulatory Toxicology and Pharmacology 28, 124-132, 1998). Drugs in a variety of therapeutic classes have shown some toxic effects in the eye. Drugs that have shown some drug-induced retinal effects include:
antimalarials, such as, for example, chloroquine, quinine;
antibiotic drugs, such as for example, aminoglycosides, sparsomycin, clioquinol;
antituberculosis drugs, such as, for example, ethambutol;
antifungal drugs, such as, for example, miconazole;
CNS drugs, such as, for example, phenothiazines, such as, for example, chlorpromazine, amitriptyline, lysergide;
cardiovascular drugs, such as, for example, nifedipine, amiodarone;
antineoplastic drugs, such as, for example, 5-fluorouracil, tamoxifen, carmustine, chlorambucil, cis-platinum, mitotane, nitrogen mustard, nitroso ureas, vinblastine, vincristine, doxorubicin;
dermatological drugs, such as, for example, etretinate, canthaxanthin, isotretinoin;
anti-inflammatory drugs, such as, for example, corticosteroids, ibuprofen, indomethacin, phenylbutazone;
immunomodulator drugs, such as, for example, tilorone (antiviral) alpha interferon;
oral contraceptives
hormones, such as, for example, clomiphene;
others, such as, for example, deferoxamine, niacin, warfarin;
sympathomimetic drugs, such as, for example, dipivefrin, phenylephrine, epinephrine.

The RPE together with the capillary wall constitutes the blood-retinal barrier. Entry of therapeutic drugs into retina and/or RPE cells in the eye is accomplished by receptor mediated transcytosis. In some embodiments, therapeutic drugs bind to a member of the LDL receptor gene family in retina and/or RPE cells in the eye and undergo receptor mediated transcytosis. Provided herein are methods and compositions for the treatment and/or prevention of retinal toxic side effects attributed to therapuetic drugs. In one embodiment, the binding of a therapuetic drug, which exhibits ocular toxicity, to a member of the LDL receptor gene family in retina and/or RPE cells is inhibited by an LDL receptor gene family binding agent, such as, for example, a megalin-binding agent. In another embodiment, a member of the LDL receptor gene family in retina and/or RPE cells is antagonized with an LDL receptor gene family binding agent, thus preventing binding and uptake of a therapuetic drug into retina and/or RPE cells. In one embodiment, the therapeutic drug is an antibiotic drug. In another embodiment, the therapeutic drug is an aminoglycoside. In some embodiments, the retinal-toxic therapuetic drug is bound to a lipoprotein or a carrier protein, such as, for example, albumin or lactoferrin. Binding of the carrier protein or lipoprotein to the member of the LDL receptor gene family in retina and/or RPE cells provides another means for entry of the retinal-toxic therapuetic drug into retina and/or RPE cells. In one embodiment, a member of the LDL receptor gene family in retina and/or RPE cells is antagonized with an LDL receptor gene family binding agent, thus preventing binding and uptake of a carrier protein or liprotein into retina and/or RPE cells.

### LDL Receptor Gene Family

Individual proteins can possess one or more discrete monomer domains. These proteins are often called mosaic proteins. For example, members of the low density lipoprotein (LDL)-receptor gene family contain four major structural domains: the cysteine rich A-domain repeats, epidermal growth factor precursor-like repeats, a transmembrane domain and a cytoplasmic domain. The LDL-receptor gene family includes the low density lipoprotein (LDL) receptor, very-low-density lipoprotein receptors (VLDL-R), apolipoprotein E receptor 2, LDL receptor-related protein (LRP) and megalin. Family members have the following characteristics: 1) cell-surface expression; 2) extracellular ligand binding consisting of A-domain repeats; 3) requirement of calcium for ligand binding; 4) recognition of receptor-associated protein and apolipoprotein (apo) E; 5) epidermal growth factor (EGF) precursor homology domain containing YWTD repeats; 6) single membrane-spanning region; and 7) receptor-mediated endocytosis of various ligands. See Hussain et al., The Mammalian Low-Density Lipoprotein Receptor Family, (1999) Annu. Rev. Nutr. 19: 141-72. Yet, the members bind several structurally dissimilar ligands.

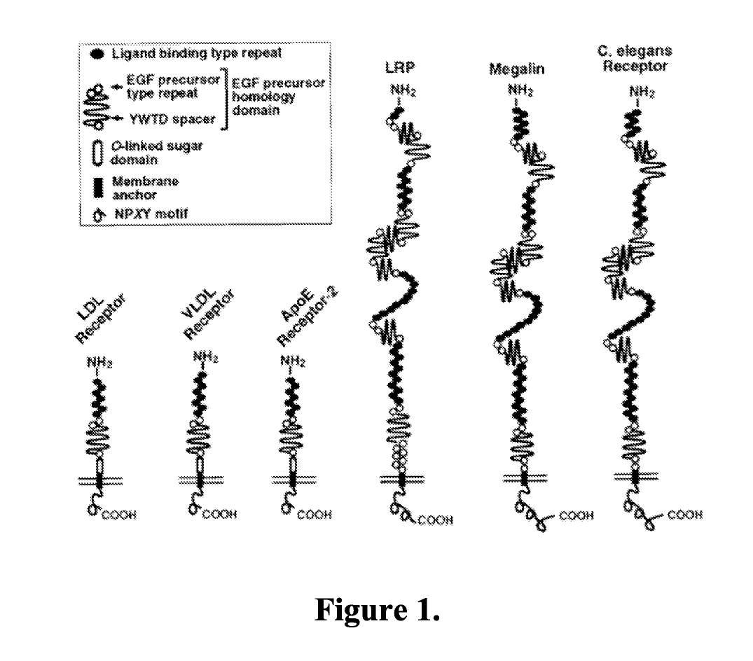

The proteins of the low density lipoprotein (LDL) receptor gene family (Neels, J. G. et al., Fibrinolysis Proteolysis 12, 219-240, 1998), are a group of related mosaic transmembrane receptors of similar structure and binding a diverse range of protein ligands in their ectodomains. Ligands bound to the any of the members of the LDL receptor gene family are internalized by classical endocytosis (Chen et al., J. Biol. Chem. 265, 3116-3123, 1990). In humans, the group of known LDL receptor gene family proteins includes, for example, the LDL receptor (Russell, D. et al., Cell 37, 577-585, 1984), the LDL receptor-related protein (LRP) (Herz, J. et al., EMBO J. 7, 4119-4127, 1988; Kristensen, T. et al., FEBS Lett. 276, 151-155, 1990), the very low density lipoprotein receptor (VLDLR) (Webb, J. C. et al. Hum. Mol. Genet. 3, 531-537, 1994), the apoe receptor2 (apoER2) (Kim et al. J. Biol. Chem. 271, 8373-8380, 1996), megalin/gp330/LRP2 (Hjaln, G. et al. Eur. J. Biochem. 239, 132-137, 1996), LRP6 (Brown et al. Biochem. Biophys. Res. Commun. 248, 879-888, 1998) and LRP7 (Hey, P. J. et al., Gene (Amst.) 216, 103-111, 1998; Dong, Y. et al., Biochem. Biophys. Res. Commun. 251, 784 790, 1998). See FIG. 1.

Members of the LDL receptor gene family are a family of single-pass type I membrane proteins that mediate uptake of various protein cargoes into cells via the endocytic pathway (Krieger, M. and Herz, J. Structures and functions of multiligand lipoprotein receptors: macrophage scavenger receptors and LDL receptor-related protein (LRP). Annu. Rev. Biochem. 63, 601-637, 1994). Each receptor binds many different cargo proteins, and continuously recycles to and from the cell surface. Some ligands can bind to different members of the LDL receptor gene family. Some ligands can bind to only one member of the LDL receptor gene family. In some embodiments, some members of the LDL receptor gene family can bind the same ligand. In other embodiments, only one member of the LDL receptor gene family can bind to a particular ligand. In other embodiments, members of the LDL receptor gene family that are expressed in different tissue types bind to the same ligand. In other embodiments, members of the LDL receptor gene family expressed in different tissue types do not bind to the same ligand. In some embodiments, members of the LDL receptor gene family that are expressed in different portions of a cell bind to the same ligand. In other embodiment, members of the LDL receptor gene family that are expressed in different portions of a cell bind to the same ligand. In some embodiments, a member of the LDL receptor gene family that is present on the basal membrane of RPE cells binds to the same ligand that also binds to a member of the LDL receptor gene family that is present on the apical membrane of RPE cells. In some embodiments, a member of the LDL receptor gene family that is present on the basal membrane of RPE cells does not bind to the same ligand that binds to a member of the LDL receptor gene family that is present on the apical membrane of RPE cells.

Members of the LDL receptor gene family are characterized as possessing five common structural motifs:
a) Ligand-binding (complement) type cysteine-rich repeats;
b) Epidermal growth factor (EGF) receptor-like cysteine rich repeats;
c) tyrosine-tryptophan-threonine-aspartic acid (YWTD) domains;
d) a single membrane-spanning segment; and
e) a cytoplasmic tail that include 1-3 NPxY motifs.

The amino-terminal region contains ligand-binding-type repeats, stretches of approximately 40 amino acids each that are characterized by three internal disulfide bonds, in clusters of between two and eleven individual repeats. Most of the ligands to these receptors interact with these ligand-binding domains. The presence of multiple ligand-binding domains leads to various modes of ligand binding to the receptors. In some members of the LDL receptor gene family, there are multiple, independent binding sites for a variety of ligands. For some ligands, there is only a single high-affinity binding site on the receptor. In some cases, two or more different ligands with different binding sites might be able to bind to the receptor simultaneously. In some cases, a receptor protein can bind numerous structurally distinct ligands with high affinity as a result of: the presence of multiple ligand-binding-type repeats in the receptor protein, the unique contour surface and charge distribution for each repeat, and from multiple interactions between both the ligand and the receptor. Some ligands can recognize different combinations of these repeats in a sequential fashion, while others appear to recognize repeats from separate clusters. It has been reported that RAP occupies two binding sites on the receptor protein megalin (Beeg, E J. Br. J. Clin. Pharmacol. 39, 597-603, 1995), whereas approximately 60-100 molecules of the low molecular weight organic compound gentamicin bind per megalin protein (Schmitz, C. J. Biol. Chem. 227, 618-622, 2002).

The ligand-binding (complement) type cysteine-rich repeats contain a number of negatively charged residues that are capable of binding to cationic ligands (see, for example, US 2003/0202974, incorporated by reference). In some embodiments, the binding of cationic ligands is accomplished by ionic interactions with the receptor protein.

Ligand binding domains are followed by cysteine-rich epidermal growth factor (EGF) precursor-type repeats, separated by cysteine-poor spacer regions. The spacer regions contain YWTD motifs responsible for pH-dependent release of ligands in endosomal compartments. YWTD motifs flanked by EGF precursor-type repeats are referred to as the EGF precursor homology domain. In LRP and gp330, EGF precursor homology domains are either followed by another ligand-binding domain or a spacer region.

In contrast to their extracellular domains, the cytoplasmic tails of the different receptors share very little sequence similarity, with the exception of a short amino acid motif characterized by the consensus sequence NPxY, which designates the tetra-amino acid motif asparagine-proline-X-tyrosine (where X represent any amino acid), which has been shown to mediate clustering of the LDL receptor in coated pits before endocytosis (Willnow T. E. et al. Nature Cell Biology, vol 1, E157-E162, 1999).

Some members of the LDL receptor gene family, such as the LDL receptor and VLDL receptor, contain an O-linked sugar domain in the extracellular space next to the single membrane-spanning segment.

Members of the LDL receptor gene family have been sequenced, such as, for example:

LRP (LDL receptor related protein; alpha-2-macroglobulin receptor)
cDNA:X13916 NM_{—}002332
gene: AH003324

LRP2 (megalin; gp330; gp600)
cDNA: U33837
gene: NT_{—}002176

apolipoprotein E receptor 2 (ApoE receptor 2; LRP8)
cDNA: D50678
gene: SEG_D86389S

very low density lipoprotein receptor (VLDL receptor)
cDNA: D16493
gene: SEG_HUMVLDLR

LRP1B
cDNA: NM_{—}018557

MGEF-7
cDNA: AB011540

### LDL Receptor

Cells take up cholesterol from the blood by the endocytosis of low-density lipoproteins (LDL) using the LDL receptor. After binding their ligand, LDL receptors cluster in the coated pits in the plasma membrane. This is then followed by the formation and internaliztion of endocytic vesicles, hydrolysis of the endocytosed lipoproteins in lysosomes and release of the lipids into the cytoplasm. (Brown et al. A receptor-mediated pathway for cholesterol homeostasis. Science. 232, 34-47 (1986)). The LDL receptor plays a key role in cholesterol homeostasis by mediating the cellular internalization of apolipoprotein B and/or apolipoprotein E (apoE) containing lipoproteins. The LDL receptor has a 50 residue cytoplasmic domain which contains an NPxY (Asn-Pro-x-Tyr, where x represents any amino acid) sequence that targets this receptor to clathrin-coated pits. The extracellular portion contains an O-linked sugar domain and two clusters of cysteine-rich repeats. The first cysteine-rich cluster, which is located next to the O-linked sugar domain, has homology with the epidermal growth factor like repeats that are separated by five copies of a repeat, each containing a common tetrapeptide, tyrosine-tryptophan-threonine-aspartic acid (YWTD). The epidermal growth factor homology appears necessary for the LDL receptor to undergo an acid-dependent conformational change that releases ligands within the endosomes, allowing unoccupied receptors to recycle back to the cell surface. The second cysteine rich cluster contains seven complement like repeats, which are responsible for binding the ligands apolipoproteins B and E.

LDL-Receptor Related Protein (LRP)

The LDL receptor related protein (LRP) is larger than but structurally similar to other members of the LDL receptor gene family. (Herz et al. J. Clin. Invest. 108:779-784, 2001; Willnow et al. Nature Cell Biology, vol. 1, E157-E162, 1999; Kreiger et al. Structures and functions of multiligand lipoprotein receptors:macrophage scavenger receptors and LDL receptor-related protein (LRP). Annu. Rev. Biochem. 63:601-637 (1994)). LRP is synthesized as a 600 kDa precursor (LRP 600) that is cleaved to generate an amino terminal 515 kDa fragment (LRP 515) and a carboxyl-terminal 85 kDa fragment (LRP85). LRP 515 harbors all known ligand binding sites and remains noncovalently associated with LRP 85, which contains the membrane anchor and the cytoplasmic domain.

As used herein, “LRP” refers to a protein whose cDNA encoding sequence has at least a 75% nucleotide identity with cDNA:X13916 NM_{—}002332; gene: AH003324.

As used herein, “LRP-related protein” refers to a protein that belongs to the LDL receptor gene family and has greater than 50% homology to LRP; or reacts with high speficity to anti-LRP antibodies (specific ones).

Whereas the LDL receptor appears to act solely in lipoprotein metabolism, LRP and other members of this family, appears to have other distinct functions. Among the many functions of LRP, it has been shown that chylomicrons are absorbed in the liver by the endocytotic action of LRP. Other LRP Binding Ligands include:
proteinases and inhibitor complexes: such, as for example, α₂M-proteinase complexes, Pregnancy Zone Protein (PZP)-proteinase complexes, t-PA, u-PA, t-PA:PAI-1, u-PA:PAI-1, uPA:proteinase nexin 1, tissue factor pathway inhibitor, elastase-α₁-antitrypsin, α₁-antitrypsin, C1 inhibitor;
lipoproteins: such as, for example, apo E, apo E-enriched β-VLDL, lipoprotein lipase, lipoprotein lipase-enriched VLDL, lipoprotein lipase-enriched β-VLDL, hepatic lipase;
blood coagulation or blood clotting agents: such as, for example, Factor IXa, Factor VIIIa, Factor VIIa/TFPI, antithrombin III, THPI, heparin cofactor II;
chaperone proteins: such as, for example, HSP-96, RAP;
matrix proteins: such as, for example, thrombospondin-1, thrombospondin-2;
other molecules: such as, for example, pseudomonas exotoxin A, lactoferrin, RAP, α2-macroglubulin, chylomicron remnants, complement C3, spingolipid activator protein (SAP), rhinovirus, HIV-Tat protein, MMP-13, MMP-9, the hormone thyrotropin, the cofactor cobalamin and the lysosomal protein saposin; RBP and iRBP.

### Megalin

Megalin, also known as gp330 or LRP2, is a 600-kDa cell surface protein in its glycosylated form, which is expressed on many epithelial surfaces of the human body including the renal proximal tubules, the cochlea of the inner ear, and the ciliary epithelium of the eye (Christensen et al. Essential Role of Megalin in Renal Proximal Tubule for Vitamin Homeostasis. J. Am. Soc. Nephrol. 10: 2224-2236, 1999). As shown herein, at least megalin protein or at least one megalin-related protein is also expressed in retina and RPE cells in the eye.

The deduced cDNA sequence from rat and human megalin encodes a protein of approximately 600 kDa, which exhibits all of the hallmarks of an endocytic receptor of the LDL receptor gene family. Megalin is a type 1 cell surface transcytosis receptor with a single transmembrane domain. Megalin belongs to low density lipoprotein (LDL) receptor gene family. Megalin is a type 1 cell surface endocytosis receptor with a single transmembrane domain, a short cytoplasmic tail, and a large amino-terminal portion extending into the extracellular space. The amino-terminal region contains ligand-binding (complement) type cysteine-rich repeats, which are stretches of approximately 40 amino acids each that are characterized by three internal disulfide bonds. These repeats constitute the binding sites for ligands, and it has been demonstrated that several ligands bind to the same or closely associated sites in the second cluster of ligand-binding repeats (Orlando RA. et al. Proc. Natl. Acad. Sci. USA 94:2368-2373, 1997). Furthermore, megalin harbors cysteine-rich epidemial growth factor (EGF) precursor-type repeats, separated by cysteine-poor spacer regions. The spacer regions contain YWTD motifs responsible for pH-dependent release of ligands in endosomal compartments. The cytoplasmic tail of megalin carries three copies of a NPxY motif, which directs receptors into coated pits. Megalin does not contain an O-linked sugar domain, which is found in some receptors of the gene family, such as, for example, the LDL receptor and VLDL receptor.

The extracellular portion of megalin and LRP resemble multiple copies of the LDL receptor domain. The overall amino acid sequence identity between megalin and other family members varies between 30 and 50%.

The sequence for the megalin receptor is shown as:
cDNA:U33837
gene:NT_{—}002176

The human megalin gene is located on chromosome 2q24-q31 (Korenberg J R. et al. Genomics 22:88-93, 1994)

Unlike the LDL receptor, whose primary role is to mediate cellular uptake of cholesterol-loaded lipoproteins, megalin, LRP and other members of the LDL receptor gene family, bind and/or recognize a variety of structurally distinct ligands with high affinity. Megalin has been shown to function as a promiscuous scavenger receptor primarily involved in uptake of proteins, lipid-soluble vitamins and steroid hormones into tissues that express the receptor. Megalin Binding ligands include a long list of diverse proteins and chemical substances.

Megalin Binding ligands include:
vitamin-binding proteins, which include, for example, transcobalamin-vitamin B12, vitamin-D-binding protein, retinol-binding protein, interphotoreceptor retinoid binding protein;
lipoproteins, which include, for example, apolipoprotein B, apolipoprotein E, apolipoprotein J/clusterin, apolipoprotein H/β₂-glycoprotein-I;
immune- and stress related proteins, which include, for example, immunoglobulin light chains, PAP-1, β2-microglobulin;
steroid hormone binding proteins, which include, for example, sex hormone binding protein-estrogens, androgen binding protein-androgens;
hormones and precursors, which include, for example, parathyroid hormone, insulin, epidermal growth factor, prolactin, thyroglobulin;
enzyme and enzyme inhibitors, which include, for example, PAI-1, PAI-1-urokinase, PAI-1-tPA, Pro-urokinase, lipoprotein lipase, plasminogen, β-amylase, β1-microglobulin, lysozyme, aprotinin;
Other carrier proteins, which include, for example, albumin, lactoferrin, hemoglobin, odorant-binding protein, transthyretin;
low molecular weight peptides and hormones, which include, for example, PTH, insulin, β2-microglobulin, epidermal growth factor, prolactin, lysozyme, cytochrome c;
Drugs and toxins, which include, for example, Aminoglycosides, gentamicin, polymyxin B, aprotinin, trichosanthin;
antibodies, which include, for example anti megalin antibody, rabbit anti-rat megalin antibody, rabbit pre-immune IgG;
Other ligands include, for example, RAP, Ca²⁺, cytochrome c, retinol, retinal, EDTA, thyroglobulin, plasminogen, albumin, lactoferrin.

Megalin interacts with its ligands through the extracellular domains of the receptor. Binding occurs either through complex protein-protein interactions (if the ligand is a protein), or through simple ionic interaction of positively charged substances with arrays of negatively charged amino acids in the complement type repeats (if the ligand is a chemical compound). Binding of lipid-soluble vitamins and steroid hormones to megalin are indirect and mediated though interaction of the receptor with specific carrier proteins that transport these substances in plasma, such as, for example, retinol binding protein or interphotoreceptor retinoid binding protein.

“Megalin,” as used herein, refers to a protein that is expressed in the retina or retinal pigment epithelial cells of a mammal, whose cDNA encoding sequence has at least a 75% nucleotide identity with either the human megalin cDNA sequence having gene accession number U04441 disclosed in Korenberg, J. R. et al. (Genomics. 1994 Jul. 1; 22(1):88-93, 1994), gene accession number U33837 disclosed in Hjalm, G., et al. (Eur J Biochem. 239(1):132-7, 1996)) or the rat megalin cDNA sequence having gene accession number L34049 disclosed in Saito et al. (Proc. Natl. Acad. Sci. USA, 91:9725-9729, 1994).

As used herein, “megalin-related protein” refers to a protein that belongs to the LDL receptor gene family and has greater than 50% homology to megalin; or reacts with high speficity to anti-megalin antibodies (specific ones);

“Megalin binding ligand” means: (1) a substance that binds with megalin, (2) a substance that is incorporated into a cell by endocytosis by a mechanism that is mediated by megalin or (3) a substance that itself binds to a substance described in (1) or (2) of this definition.

The term “endogenous megalin binding ligand” means a megalin binding ligand that originates or is produced within a mammal.

“Nucleotide identity” means the sequence alignment of a nucleotide sequence calculated against another nucleotide sequence, e.g. the nucleotide sequence of human megalin. Specifically, the term refers to the percentage of residue matches between at least two nucleotide sequences aligned using a standardized algorithm Such an algorithm may insert gaps in the sequences being compared in a standardized and reproducible manner in order to optimize alignment between the sequences, thereby achieving a more meaningful comparison. Percent identity between nucleotide sequences is preferably determined using the default parameters of the CLUSTAL W algorithm as incorporated into the version 5 of the MEGALIGN™ sequence alignment program. This program is part of the LASERGENE™ suite of molecular biological analysis programs (DNASTAR, Madison Wis.). CLUSTAL W is described in Thompson 1994).

“Nucleotide sequence” and “polynucleotide” refer to DNA or RNA, whether in single-stranded or double-stranded form. The term “complimentary nucleotide sequence” refers to a nucleotide sequence that anneals (binds) to a another nucleotide sequence according to the pairing of a guanidine nucleotide (G) with a cytidine nucleotide (C) and adenosine nucleotide (A) with thymidine nucleotide (T), except in RNA where a T is replaced with a uridine nucleotide (U) so that U binds with A.

Members of the LDL receptor gene family are expressed in different tissue types. Members of the LDL receptor gene family are expressed in retina and RPE cells, as well as in the kidney. Megalin is expressed in retina and RPE cells in the eye, as well as in the kidney.

### Cubilin

Cubilin, a 460 kDa membrane-associated protein colocalizing with megalin in some tissue types, may facilitate the endocytic process by sequestering a ligand on the cellular surface before megain-mediated internalization of the cubilin-bound ligand. The ligand may bind to cubilin as well as directly to megalin. Cubilin, however, appears not to be able to mediated endocytosis on its own but megalin can physically associate with cubilin and mediate its internalization.

The sequence of cubilin is shown as:
cDNA:XM_{—}011904
gene:NT_{—}008682 (Homo sapiens chromosome 10 working draft sequence segment)

### Receptor-Associated Protein (RAP)

The normal processing of LRP, megalin and other members of the LDL receptor gene family requires the presence of RAP, a 39 kDa protein (Bu, G. et al., J. Biol. Chem. 271, 22218-22224, 1996; Strickland, D. K. e t al., J. Biol. Chem. 266, 13364-13369, 1991). RAP appears to consist of three homologous domains (Bu, G., et al., EMBO J. 14, 2269-2280, 1995; Ellgaard, L. et al., Eur. J. Biochem. 244, 544-551, 1997; Rall, S. C. et al., J. Biol. Chem. 273, 24152-24157, 1998; Medved, L. V. et al., J. Biol. Chem. 274, 717-727, 1999.) of which domain 1 has been shown to consist of a three-helix bundle (Nielsen, P. R. et al., Proc. Natl. Acad. Sci. U.S.A. 94, 7521-7525, 1997). RAP interacts with all members of the LDL receptor gene family and is a universal antagonist for all receptor/ligand interactions. RAP domains 1 and 3 (RAPd3) are both receptor-binding (Warshawsky, I. et al. J. Biol. Chem. 268, 22046-22054, 1993), but only domain 3 is sufficient to mimic the chaperone-like functions of RAP in cells (Obermoeller, L. M. et al., J. Biol. Chem. 272, 10761-10768, 1997; Savonen, R. et al., J. Biol. Chem. 274, 25877-25882, 1999.). RAP domain 2 is a substrate for cAMP-dependent protein kinase (Petersen, C. M. et al., EMBO J. 15, 4165-4173, 1996) but has only a very low affinity for LRP and megalin compared with RAP domains 1 and 3 (Tauris, J. et al., FEBS Lett. 429, 27-30, 1998.). The autonomous regions of human RAP include domain 1 (amino acid positions 18-112), domain 2 (amino acid positions 113-218) and domain 3 (amino acid positions 219-323).

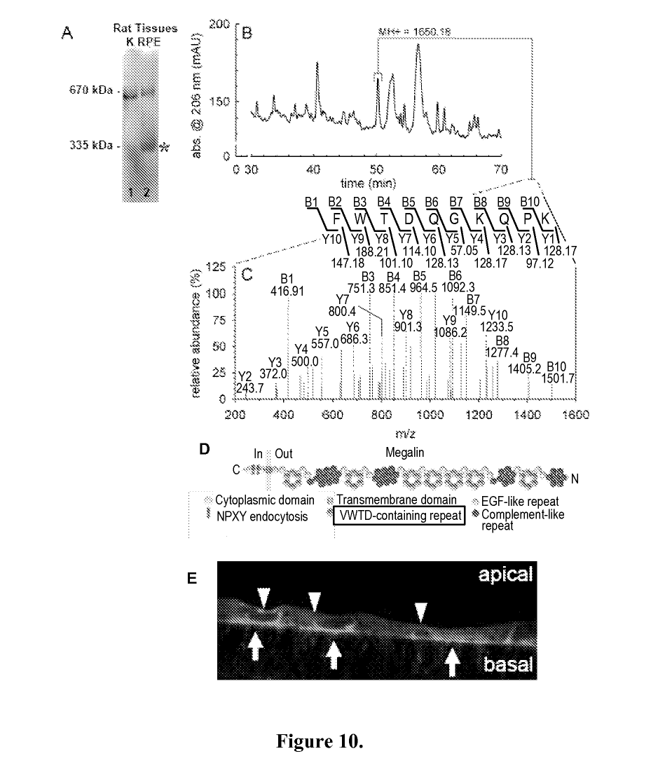

RAP has been shown to have a sequence shown in: XM_{—}003315, Gene: AH006949. RAP binds with high affinity to LRP (KD=4 nM) and antagonizes the ligand binding properties of this receptor, preventing it from mediating the cellular internalization of ligands (Williams et al. A novel mechanism for controlling the activity of α2-macroglobulin receptor/low density lipoprotein receptor-related protein. Multiple regulatory sites for 39-kDa receptor-associated protein. J. Biol. Chem. 267, 9035-9040, 1992). LRP contains multiple ligand binding sites, each independently regulated by RAP. RAP also binds with high affinity to gp330 (KD=8 nM) (Kounnas et al. The 39 kDa receptor-associated protein interacts with two members of the low density lipoprotein receptor family, α2-macroglobulin receptor and glycoprotein gp330. J. Biol. Chem. 267, 21162-21166, 1992) and the VLDL receptor (KD=0.7 nM) (Battey et al. The 39 kDa receptor-associated protein regulates ligand binding by the very low density lipoprotein receptor. J. Biol. Chem. 269, 23268-23273, 1994), but with lower affinity to the LDL receptor (KD=500 nM), and antagonizes the ligand binding properties of these receptors as well. Exogeneously added RAP or overexpression of RAP both inhibit the acitivity of members of the LDL receptor gene family (Willnow et al. Inhibition of chylomicron remnant uptake by gene transfer of a receptor anatagonist. Science, 264, 1471-1474, 1994). See also, FIG. 10, which shows that exogenous RAP inhibits the uptake of RBP-retinol in RPE cells.

It is possible to isolate minimal domains of RAP, such as peptides, that carry out the minimal functional domains of the receptor binding and inhibition and thus also function as antagonists of the members of the LDL receptor gene family. In one embodiment, a RAP derived substance is a peptide that includes a minimal functional domain having at most 104 amino acids, preferably from 20 to 60 amino acids. In particular, they are minimal functional protein domains. These peptides have at the most 104 amino acids, preferably from 20 to 60 amino acids. A preferred domain is amino acid positions 219-323 of RAP. Another preferred domain is amino acid positions 18-112 of RAP.

Gene knock-out studies have shown that cells lacking RAP exhibit a reduction in the expression of members of the LDL receptor gene family, presumably because RAP prevents premature binding of newly synthesized ligands to members of the LDL receptor gene family and precipitation of the receptor within the endoplasmic reticulum (ER). In a mouse model with an induced RAP gene defect (knockout mouse), uptake of therapeutic agents is reduced up to 50% compared to mouse models not possessing the RAP gene defect. (Willnow et al. Proc. Natl. Acad. Sci. 92:4537-4541, 1995). In one embodiment, a method for evaluating whether members of the LDL receptor gene family in the retina and RPE are responsible for cellular uptake of ligands and/or agents contemplated herein, which includes:
Administering a ligand or agent to a RAP gene defective mouse (knockout mouse)
Administering a ligand or agent to a wild-type mouse (not possesing the RAP gene defect)
Evaluating the amount of ligand or agent in retina and/or RPE cells of the animal model and control animal

In this model, the contribution of the processes responsible for uptake of the ligands or agents into the retina and/or RPE cells carried out by the members of the LDL receptor gene family may be quantified. The amount of intracellular accumulation of the ligand or agent in the RAP gene defective mice as compared to RAP sufficient mice indicates whether the mechanism of intracellular accumulation is mediated by members of the LDL receptor gene family or by some other mechanism.

The above-mentioned experiment may also be carried out by using a mouse model with an induced megalin gene defect (knockout mouse; Nykjaer et al. Cell, 96, 507-515). In these animal models, the contribution of megalin or other receptor-mediated or receptor-independent processes to the ligand or agent uptake into the retina and RPE cells may be tested. The amount of intracellular accumulation of the ligand or agent as compared to a control having sufficient megalin indicates whether the mechanism of intracellular accumulation is through megalin binding or some other mechanism.

In some embodiments, the ligand is retinol, RBP-retinol complex, or RBP-retinol-TTR complex. In some embodiments, the ligand is IRBP, IRBP-retinol, or IRBP-retinal. In some embodiments, the ligand is a drug or toxin. In another embodiment, the ligand is an antiobiotic. In another embodiment, the ligand is an aminoglycoside.

In some embodiments, agents contemplated herein can be conjugated to RAP or a RAP polypeptide, in the diagnosis, prophylaxis, or treatment of diseases and conditions associated with the retina and RPE cells, see, for example, US 20060029609, which is incorporated by reference.

### Chemical and Biochemical Terminology

Unless defined otherwise, all technical and scientific terms used herein have the same meaning as is commonly understood by one of skill in the art to which the claimed subject matter belongs. Unless stated otherwise, the claim terms and chemical terms used herein are as defined in U.S. patent application Ser. No. 11/150,641, filed Jun. 10, 2005, which is incorporated by reference herein for that purpose. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that both the foregoing general description and the following detailed description are exemplary and explanatory only and are not restrictive of the subject matter claimed. In this application, the use of the singular includes the plural unless specifically stated otherwise. In this application, the use of “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester derivatives as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base- protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

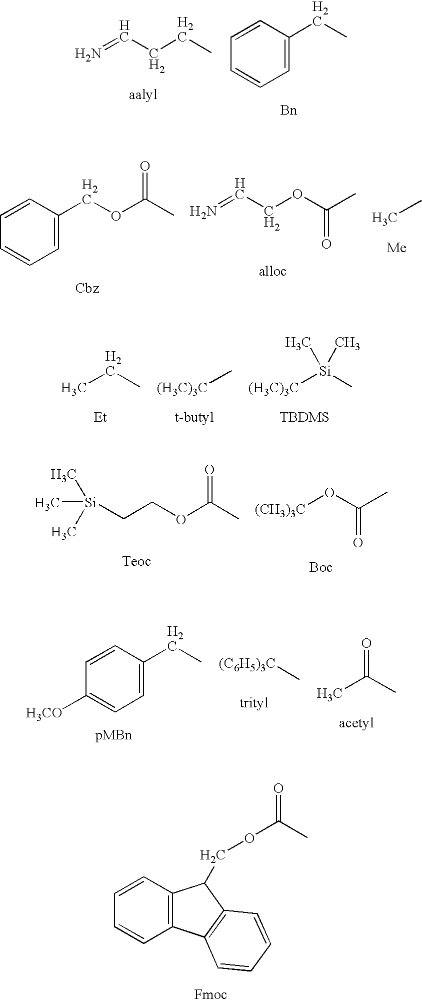

Other protecting groups are described in Greene and Wuts, Protective Groups in Organic Synthesis, 3rd Ed., John Wiley & Sons, New York, N.Y., 1999, which is incorporated herein by reference in its entirety.

The term “optionally substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. The protecting groups that may form the protective derivatives of the above substituents are known to those of skill in the art and may be found in references such as Greene and Wuts, above.

The compounds presented herein may possess one or more chiral centers and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of an agent that modulates the activity of a member of the LDL receptor gene family, such as, for example, a Megalin-modulating agent, as well as active metabolites of these compounds having the same type of activity. In some situations, compounds may exist as tautomers. All tautomers are included within the scope of the compounds presented herein. In addition, the agent that modulate members of the LDL receptor gene family described herein can exist in unsolvated as well as solvated forms with pharmaceutically acceptable solvents such as water, ethanol, and the like. The solvated forms of the compounds presented herein are also considered to be disclosed herein.

As used herein, the amino acids, which occur in the various amino acid sequences appearing herein, are identified according to their well-known, three-letter or one-letter abbreviations. The nucleotides, which occur in the various DNA fragments, are designated with standard single-letter designations used routinely in the art (see, Table 1).

As used herein, amino acid residue refers to an amino acid formed upon chemical digestion (hydrolysis) of a polypeptide at its peptide linkages. The amino acid residues described herein are, in certain embodiments, in the “L” isomeric form. Residues in the “D” isomeric form can be substituted for any “L” amino acid residue, as long as the a desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552 59 (1969) and adopted at 37 C.F.R. §§ 1.821-1.822, abbreviations for amino acid residues are shown in the following Table:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino terminus to carboxyl terminus. In addition, the phrase “amino acid residue” is broadly defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino terminal group such as NH₂ or to a carboxyl terminal group such as COOH.

In a peptide or protein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

Such substitutions can be made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Asp (D) | Glu |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and can be determined empirically or in accord with known conservative substitutions.

As used herein, the term “selective binding compound” refers to an agent that selectively binds to any portion of one or more target receptors.

As used herein, the term “selectively binds” refers to the ability of a selective binding agent to bind to a target receptor with greater affinity than it binds to a non-target receptor. In certain embodiments, specific binding refers to binding to a target with an affinity that is at least 10, 50, 100, 250, 500, 1000 or more times greater than the affinity for a non-target.

As used herein, the term “target receptor” refers to a receptor or a portion of a receptor capable of being bound by a selective binding compound. In certain embodiments, a target receptor is a member of the LDL receptor gene family. In some embodiments, the target receptor is a retinoid binding protein receptor. In some embodiments, the retinoid binding protein receptor is a member of the LDL receptor gene family.

As used herein, “agent” refers to any substance that is capable of interacting with a member of the LDL receptor gene family, thereby modulating the activity of said receptor protein.

As used herein, the term “modulator” refers to a compound that alters an activity of a molecule. For example, a modulator can cause an increase or decrease in the magnitude of a certain activity of a molecule, such as, for example, a member of the LDL receptor gene family, compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

An agent which modulates a biological activity of a subject polypeptide, such as a member of the LDL receptor gene family, increases or decreases the activity at least about 10%, at least about 15%, at least about 20%, at least about 25%, at least about 50%, at least about 100%, or at least about 2-fold, at least about 5-fold, or at least about 10-fold or more when compared to a suitable control.

The term “ligand” refers to any molecule that binds to a specific site on another molecule, such as, for example, a member of the LDL receptor gene family.

The term “endogenous ligand” or “endogenous binding ligand” means a ligand that originates or is produced within a mammal.

The term “modulate” encompasses an increase or a decrease, a stimulation, inhibition, or blockage in the measured activity when compared to a suitable control.

An agent that “modulates the level of expression of a nucleic acid” in a cell is one that brings about an increase or decrease of at least about 1.25-fold, at least about 1.5-fold, at least about 2-fold, at least about 5-fold, at least about 10-fold, or more in the level (i.e., an amount) of mRNA and/or polypeptide following cell contact with a candidate agent compared to a control lacking the agent.

Agents that bind to members of the LDL receptor gene family in retina and RPE cells will generally have a greater affinity to the receptor protein than a naturally occurring ligand, such as, for example retinoid binding protein. The agent will have at least 2 times greater affinity to the member of the LDL receptor gene family in retina and RPE cells than retinoid binding protein. In another embodiment, the agent will have at least 5 times greater affinity to the member of the LDL receptor gene family in retina and RPE cells than a retinoid binding protein. In another embodiment, the agent will have at least 10 times greater affinity for a member of the LDL receptor gene family in retina and RPE cells than a retinoid binding protein. Affinity for the receptor is measured by standard methods known in the art.

As used herein, “retinoid binding protein” refers to any carrier protein that is able to bind to retinoids. Unless specifically designating a particular retinoid binding protein, retinoid binding proteins include, for example, retinol-binding protein (RBP), interstitial retinoid binding protein (IRBP), retinaldehyde-binding protein (RALBP), cellular retinol-binding protein (CRBP), and cellular retinaldehyde-binding protein (CRALBP).

As used herein, “interphotoreceptor retinoid binding protein” and “interstitial retinol binding protein” are used interchangeably and refer to the same protein.

As used herein, the term “receptor mediated activity” refers any biological activity that results, either directly or indirectly, from binding of a ligand to a receptor.

As used herein, the term “agonist” refers to a compound, the presence of which results in a biological activity of a receptor that is the same as the biological activity resulting from the presence of a naturally occurring ligand for the receptor.

As used herein, the term “partial agonist” refers to a compound the presence of which results in a biological activity of a receptor that is of the same type as that resulting from the presence of a naturally occurring ligand for the receptor, but of a lower magnitude.

As used herein, the term “antagonist” refers to a compound, the presence of which results in a decrease in the magnitude of a biological activity of a receptor. In certain embodiments, the presence of an antagonist results in complete inhibition of a biological activity of a receptor.

As used herein, the IC₅₀ refers to an amount, concentration or dosage of a particular test compound that achieves a 50% inhibition of a maximal response, such as modulation of androgen receptor activity, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

The terms “polypeptide,” “peptide,” and “protein,” used interchangeably herein, refer to a polymeric form of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic, or depsibicyclic peptide backbones. The term also includes conjugated proteins, fusion proteins, including, but not limited to, GST fusion proteins, fusion proteins with a heterologous amino acid sequence, fusion proteins with heterologous and homologous leader sequences, fusion proteins with or without N-terminal methionine residues, pegylated proteins, and immunologically tagged proteins. Also included in this term are variations of naturally occurring proteins, where such variations are homologous or substantially similar to the naturally occurring protein, as well as corresponding homologs from different species. Variants of polypeptide sequences include insertions, additions, deletions, or substitutions compared with the subject polypeptides. The term also includes peptide aptamers.

As used herein, the term “tissue-selective” refers to the ability of an agent to modulate a biological activity in one tissue to a greater or lesser degree than it modulates a biological activity in another tissue. The biological activities in the different tissues can be the same or they can be different. The biological activities in the different tissues can be mediated by the same type of target receptor. For example, in certain embodiments, a tissue-selective compound can modulate biological activity associated with a member of the LDL receptor gene family in one tissue and fail to modulate, or modulate to a lesser degree, biological activity associated with a member of the LDL receptor gene family in another tissue type.

An “active fragment” is a fragment having structural, regulatory, or biochemical functions of a naturally occurring molecule or any function related to or associated with a metabolic or physiological process. For example, a fragment demonstrates activity when it participates in a molecular interaction with another molecule, when it has therapeutic value in alleviating a disease condition, or when it has prophylactic value in preventing or reducing the occurrence of disease, or when it induces an immune response to the molecule. Active polypeptide fragments include those exhibiting activity similar, but not necessarily identical, to an activity of a polypeptide set forth herein. The activity may include an improved desired activity, or a decreased undesired activity.

“Expression” of a nucleic acid molecule refers to the conversion of the information into a gene product. A gene product can be the direct transcriptional product of a gene (e.g., mRNA, tRNA, rRNA, antisense RNA, ribozyme, structural RNA, or any other type of RNA) or a protein produced by translation of an mRNA. Gene products also include RNAs that are modified, e.g., by processes such as capping, polyadenylation, methylation, and editing, and proteins modified by, for example, methylation, acetylation, phosphorylation, ubiquitination, ADP-ribosylation, myristilation, and glycosylation.

A “gene,” for the purposes of the present disclosure, includes a DNA region encoding a gene product, as well as all DNA regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

The term “antibody” refers to protein generated by the immune system that is capable of recognizing and binding to a specific antigen. Antibodies, and methods of making antibodies, are commonly known in the art.

As used herein, the term “antibody” encompasses polyclonal and monoclonal antibody preparations, as well as preparations including hybrid antibodies, altered antibodies, chimeric antibodies and, humanized antibodies, as well as: hybrid (chimeric) antibody molecules; F(ab′)₂ and F(ab) fragments; Fv molecules (noncovalent heterodimers; single-chain Fv molecules (sFv); dimeric and trimeric antibody fragment constructs; minibodies; humanized antibody molecules; and, any functional fragments obtained from such molecules, wherein such fragments retain specific-binding.

An “antigen” is a substance that provokes an immune response.

An “epitope” is the site of an antigenic molecule to which an antibody binds.

An “agonist antibody” is one that mimics, enhances, stimulates, or activates the function of a molecule with which the agonist interacts.

An “antagonist antibody” is one that competes, inhibits, or interferes with the activity of a molecule with which the antagonist interacts. For example, an antagonist antibody may bind to the receptor without inducing an active response.

An “antigen-binding fragment (Fab fragment)” is a disulfide-linked heterodimer, each chain of which contains one immunoglobulin constant region (C) domain and one variable region (V) domain; the juxtaposition of the V domains forms the antigen-binding site. The two Fab fragments of an intact immunoglobulin molecule correspond to its two arms, which typically contain light chain regions paired with the V and Cl domains of the heavy chains.

A “Fragment crystallizable fragment (Fc fragment)” is the portion of an antibody molecule that interacts with effector molecules and cells. It includes the carboxy-terminal portions of the immunoglobulin heavy chains. The functional differences between heavy-chain isotypes lie mainly in the Fc fragment.

The “constant region” of an antibody is its effector region, and determines the functional class of the antibody. The constant region of a heavy or light chain is located at or near the carboxyl terminus.

The “variable region” of an antibody is the region that binds to the antigen; it provides antibody specificity. The variable region of a heavy or light chain is located at or near the amino terminus. A “VH” ftagment contains the variable region of a heavy chain; a “VL” fragment contains the variable region of a light chain.

An “immunoglobulin” is an antibody molecule.

A “heavy chain” is the larger of the two classes of polypeptide chains that combine to form immunoglobulin molecules. The class of the heavy chain determines the class of the immunoglobulin, e.g., IgG, IgA, IgE, IgD, or IgM.

A “light chain” is the smaller of the two classes of polypeptide chains that combine to form immunoglobulin molecules. Light chains are generally classified into two classes, kappa and lambda, on the basis of structural differences in their constant regions.

The “complementarity-determining region (cdr)” is the three dimensional structure of an antibody that provides antigenic specificity.

A “framework fragment” is that region of the variable domain that contains relatively invariant sequences and lies between the hypervariable regions. Framework regions provide a protein scaffold for the hypervariable regions.

A “humanized” antibody is an antibody that contains mostly human immunoglobulin sequences. This term is generally used to refer to a non-human immunoglobulin that has been modified to incorporate portions of human sequences, and may include a human antibody that contains entirely human immunoglobulin sequences.

A “single chain antibody” is a Fab fragment that includes only the V domain of a heavy chain linked by a peptide to a V domain of a light chain.

A “polyclonal antibody” a mixture of antibodies of different specificities, as in the serum of an animal immunized to various antigens or epitopes.

A “monoclonal antibody” is an antibody composition having a homogeneous antibody population. The term is not limited with regard to the species or source of the antibody, nor by the manner in which it is made. The term encompasses whole immunoglobulins and immunoglobulin fragments.

Methods of making polyclonal and monoclonal antibodies are known in the art. Polyclonal antibodies are generated by immunizing a suitable animal, such as a mouse, rat, rabbit, sheep or goat, with an antigen of interest, such as a stem cell transformed with a gene encoding an antigen. In order to enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as toxoid from diphtheria, tetanus, cholera, etc., in order to enhance the immunogenicity thereof.

The term “binds specifically,” in the context of antibody binding, refers to high avidity and/or high affinity binding of an antibody to a specific polypeptide, or more accurately, to an epitope of a specific polypeptide. Antibody binding to such epitope on a polypeptide can be stronger than binding of the same antibody to any other epitopes, particularly other epitopes that can be present in molecules in association with, or in the same sample as the polypeptide of interest. For example, when an antibody binds more strongly to one epitope than to another, adjusting the binding conditions can result in antibody binding almost exclusively to the specific epitope and not to any other epitopes on the same polypeptide, and not to any other polypeptide, which does not comprise the epitope. Antibodies that bind specifically to a subject polypeptide may be capable of binding other polypeptides at a weak, yet detectable, level (e.g., 10% or less of the binding shown to the polypeptide of interest). Such weak binding, or background binding, is readily discernible from the specific antibody binding to a subject polypeptide, e.g., by use of appropriate controls. In general, antibodies of the invention bind to a specific polypeptide with a binding affinity of 10⁻⁷ M or greater (e.g., 10⁻⁸ M, 10⁻⁹ M, 10⁻¹⁰ M, 10⁻¹¹ M, etc.).

A “disease” is a pathological, abnormal, and/or harmful condition of an organism. The term includes conditions, syndromes, and disorders.

“Treatment,” “treating,” and the like, as used herein, refer to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse affect attributable to the disorder. That is, “treatment” includes (1) preventing the disorder from occurring or recurring in a subject who may be predisposed to the disorder but has not yet been diagnosed as having it, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter.

As used herein, “fragment” is intended a polypeptide, e.g., protein domains, consisting of only a part of the intact full-length protein sequence and structure. The fragment can include a C-terminal deletion, an N-terminal deletion, and/or an internal deletion of the native polypeptide. A fragment of a protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence.

As noted above, a “biologically active” entity, or an entity having “biological activity,” is one having structural, regulatory, or biochemical functions of a naturally occurring molecule or any function related to or associated with a metabolic or physiological process. Biologically active polypeptide fragments are those exhibiting activity similar, but not necessarily identical, to an activity of a full-length polypeptide. The biological activity can include an improved desired activity, or a decreased undesirable activity. For example, an entity demonstrates biological activity when it participates in a molecular interaction with another molecule, or when it has therapeutic value in alleviating a disease condition, or when it has prophylactic value in inducing an immune response to the molecule, or when it has diagnostic value in determining the presence of the molecule. A biologically active polypeptide or fragment thereof includes one that can participate in a biological reaction, for example, as a transcription factor that combines with other transcription factors for initiation of transcription, or that can serve as an epitope or immunogen to stimulate an immune response, such as production of antibodies, or that can transport molecules into or out of cells, or that can perform a catalytic activity, for example polymerization or nuclease activity, or that can participate in signal transduction by binding to receptors, proteins, or nucleic acids, activating enzymes or substrates.

An “isolated,” “purified,” or “substantially isolated” polypeptide, or a polypeptide in “substantially pure form,” in substantially purified form,” in “substantial purity,” or as an “isolate,” is one that is substantially free of the materials with which it is associated in nature or other polypeptide sequences that do not include a sequence or fragment of the subject polypeptides. By substantially free is meant that less than about 90%, less than about 80%, less than about 70%, less than about 60%, or less than about 50% of the composition is made up of materials other than the isolated polypeptide. Where at least about 99% of the total macromolecules is the isolated polypeptide, the polypeptide is at least about 99% pure, and the composition comprises less than about 1% contaminant. Such isolated polypeptides may be recombinant polypeptides, modified, tagged and fusion polypeptides, and chemically synthesized polypeptides, which by virtue or origin or manipulation, are not associated with all or a portion of the materials with which they are associated in nature, are linked to molecules other than that to which they are linked in nature, or do not occur in nature.

Detection methods provided herein can be qualitative or quantitative. Thus, as used herein, the terms “detecting,” “identifying,” “determining,” and the like, refer to both qualitative and quantitative determinations, and include “measuring.” For example, detection methods include methods for detecting the presence and/or level of polynucleotide or polypeptide in a biological sample, and methods for detecting the presence and/or level of biological activity of polynucleotide or polypeptide in a sample.

“Biological sample,” as used herein, includes biological fluids such as blood, serum, plasma, urine, cerebrospinal fluid, tears, saliva, lymph, dialysis fluid, lavage fluid, semen, and other liquid samples or tissues of biological origin. It includes cells or cells derived therefrom and the progeny thereof, including cells in culture, cell supernatants, and cell lysates. It includes organ or tissue culture derived fluids, tissue biopsy samples, tumor biopsy samples, stool samples, and fluids extracted from physiological tissues. Cells dissociated from solid tissues, tissue sections, and cell lysates are included. The definition also includes samples that have been manipulated in any way after their procurement, such as by treatment with reagents, solubilization, or enrichment for certain components, such as polynucleotides or polypeptides. Also included in the term are derivatives and fractions of biological samples. A biological sample can be used in a diagnostic or monitoring assay.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Nucleic acid molecules are linear polymers of nucleotides, linked by 3′,5′ phosphodiester linkages. In DNA, deoxyribonucleic acid, the sugar group is deoxyribose and the bases of the nucleotides are adenine, guanine, thymine and cytosine. RNA, ribonucleic acid, has ribose as the sugar and uracil replaces thymine. Also included in the term “nucleic acid” are analogs of nucleic acids such as peptide nucleic acid (PNA), phosphorothioate DNA, and other such analogs and derivatives or combinations thereof.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a methylphosphonate diester bond, a phosphorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond, or the like, and is capable of being extended by a polymerase. Peptide nucleic acid sequences can be prepared using well known methods (see, for example, Weiler et al. (1997) Nucleic acids Res. 25:2792-2799).

A polynucleotide can be a portion of a larger nucleic acid molecule, for example, a portion of a gene, which can contain a polymorphic region, or a portion of an extragenic region of a chromosome, for example, a portion of a region of nucleotide repeats such as a short tandem repeat (STR) locus, a variable number of tandem repeats (VNTR) locus, a microsatellite locus or a minisatellite locus. A polynucleotide also can be single stranded or double stranded, including, for example, a DNA-RNA hybrid, or can be triple stranded or four stranded. Where the polynucleotide is double stranded DNA, it can be in an A, B, L or Z configuration, and a single polynucleotide can contain combinations of such configurations.

As used herein, a DNA or nucleic acid homolog refers to a nucleic acid that includes a preselected conserved nucleotide sequence, such as a sequence encoding a therapeutic polypeptide. By the term “substantially homologous” is meant having at least 80%, at least 90% or at least 95% homology therewith or a less percentage of homology or identity and conserved biological activity or function.

The terms “homology” and “identity” are often used interchangeably. In this regard, percent homology or identity can be determined, for example, by comparing sequence information using a GAP computer program. The GAP program uses the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (e.g., nucleotides or amino acids) that are similar, divided by the total number of symbols in the shorter of the two sequences. The default parameters for the GAP program can include: (1) a unary comparison matrix (containing a value of 1 for identities and 0 for non identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353 358 (1979); (2) a penalty of 3.0 for each gap and an additional 0.10 penalty for each symbol in each gap; and (3) no penalty for end gaps.

Whether any two nucleic acid molecules have nucleotide sequences that are at least 80%, 85%, 90%, 95%, 96%, 97%, 98% or 99% “identical” can be determined using known computer algorithms such as the “FASTA” program, using for example, the default parameters as in Pearson and Lipman, Proc. Natl. Acad. Sci. USA 85:2444 (1988). Alternatively the BLAST function of the National Center for Biotechnology Information database can be used to determine identity.

In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec. Biol. 215:403 (1990)).

Therefore, as used herein, the term “identity” represents a comparison between a test and a reference polypeptide or polynucleotide. For example, a test polypeptide can be defined as any polypeptide that is 90% or more identical to a reference polypeptide.

As used herein, the term at least “90% identical to” refers to percent identities from 90 to 99.99 relative to the reference polypeptides. Identity at a level of 90% or more is indicative of the fact that, assuming for exemplification purposes a test and reference polypeptide length of 100 amino acids are compared. No more than 10% (e.g., 10 out of 100) amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, or deletions.

The term substantially identical or substantially homologous or similar varies with the context as understood by those skilled in the relevant art and generally means at least 60% or 70%, preferably means at least 80%, 85% or more preferably at least 90%, and most preferably at least 95% identity.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers. In such instances, further purification might increase the specific activity of the compound.

Synthesis of Certain Agents that Modulate the Activity of Members of the LDL Receptor Gene Family

The synthesis of agents that modulate the activity of members of the LDL receptor gene family, for example, retinoid binding protein receptors, may be synthesized using standard synthetic techniques known to those of skill in the art or using methods known in the art in combination with methods described herein. See, e.g., U.S. Patent Application Publication 2004/0102650; Urn, S. J., et al., Chem. Pharm. Bull., 52:501-506 (2004). In addition, several of the agents that modulate the activity of members of the LDL receptor gene family may be purchased from various commercial suppliers. As a further guide the following synthetic methods may also be utilized.

In some embodiments, members of the LDL receptor gene family bind similar ligands. In some embodiments, members of the LDL receptor gene family that are expressed in different tissues or cells are able to bind to the same ligands or agents. In some embodiments, an agent that interacts with a member of the LDL receptor gene family in a tissue or cell other than the retina and/or RPE cells, is also able to interact with a member of the LDL receptor gene family in retina and/or RPE cells. Agents capable of interacting with member of the LDL receptor gene family are known in the art and are contemplated herein. For example, US 2003/0202974, WO 06/037335, WO 03/080103, US 2004/0198705, WO 04/084876, US 2006/0029609, US 2005/0026823, US 2005/0100986, US 2005/0089932, US 2005/0042227, US 2004/0204357, US 2004/0198705, US 2004/0049010, US 2003/0202974, US 2003/0181660, US 2003/0082640, US 2003/0157561 and US 2003/0077672 (all incorporated by reference).

The agents, which modulate the activity of members of the LDL receptor gene family in the retina and/or retinal pigment epithelium cells in the eye are preferably identified by the methods outlined herein. The agents could, for example, be antibodies, polypeptides, nucleic acids, polynucleic acids, polymers, endogenous binding ligands, low molecular weight organic compounds, Ca²⁺ scavengers, reducing agents, and fragments and derivatives of any of these.

In one embodiment the agent competitively inhibits the binding or complexing of a retinol to a retinol binding protein (RBP), or retinol to or an interphotoreceptor retinoid binding protein (IRBP). Such a compound could, for example, be a compound that specifically interacts with either the retinol compound or with the retinol binding protein or with the interphotoreceptor retinoid binding protein in a way that sterically inhibits further association with either the retinol compound or with the retinoid binding protein or with the interphotoreceptor retinoid binding protein. (see, for example, US patent publication No. 2006/0094063, incorporated by reference).

In another embodiment, the agent competitively inhibits the binding of a retinoid binding protein or interphotoreceptor retinoid binding protein to a member of the LDL receptor gene family in retina and/or retinal pigment epithilial cells. Such an agent could for example be a compound that specifically interacts with retinoid binding protein or interphotoreceptor retinoid binding protein or with the member of the LDL receptor gene family in retina and/or retinal pigment epithelial cells in a way that sterically inhibits association or further association of either the retinoid binding protein or the interphotoreceptor retinoid binding protein with the member of the LDL receptor gene family in retina and/or retinal pigment epithelial cells.

In yet another embodiment, the agent competitively inhibits the binding of a retinoid binding protein to a co-receptor of a member of the LDL receptor gene family in retina and/or retina pigment epithelial cells.

In another embodiment, the agent inhibits the binding of a retinoid binding protein to a member of the LDL receptor gene family either by blocking a sufficient amount of binding sites on the receptor protein, and/or blocking the retinoid binding protein so that it maintains the normal therapuetic effect but is inhibited from binding to the receptor protein in retina and/or RPE cells. In some embodiments, the agent is able to bind to a sufficient amount of binding sites on the receptor protein in retina and/or RPE cells, thereby inhibiting binding of the retinoid binding protein to the receptor proteins in retina and/or RPE cells. In some embodiments, the agent is able to bind to the receptor protein in retina and/or RPE cells and therefore inhibit the binding of a retinoid binding protein to the receptor protein. In some embodiments, the agent is able to bind to the retinoid binding protein, thereby preventing it from binding to a receptor protein in retina and RPE cells.

In another embodiment, the agent competitively inhibits the binding of a therapuetic drug to a member of the LDL receptor gene family in retina and/or retinal pigment epithilial cells. In another embodiment, the agent competitively inhibits the binding of an antibiotic drug to a member of the LDL receptor gene family in retina and/or retinal pigment epithilial cells. In another embodiment, the agent competitively inhibits the binding of an aminoglycoside drug to a member of the LDL receptor gene family in retina and/or retinal pigment epithilial cells.

In a still further embodiment, the agent increases the uptake of the retinoid in the retina and/or retinal pigment epithelial cells. In a further embodiment, the agent increases the binding of RBP or IRBP to the member of the LDL receptor gene family.

In another embodiment, the agent prevents the binding of retinol, RBP, RBP-retinol complex, IRBP, IRBP-retinol, TTR or RBP-retinol-TTR to the member of the LDL receptor gene family in retina and/or RPE cells.

In another embodiment, the agent prevents the uptake of retinoid in retina and/or RPE cells.

In another embodiment, the agent prevents the uptake of a therapuetic drug in retina and/or RPE cells.

In another embodiment, the agent prevents the uptake of an antibiotic drug in retina and/or RPE cells. In another embodiment, the agent prevents the uptake of an aminoglycoside drug in retina and/or RPE cells. In another embodiment, the agent prevents the uptake of gentamicin in retina and/or RPE cells.

In still another embodiment, the agent has the potential to alter the expression of a member of the LDL receptor gene family in retina and/or retinal pigment epithelial cells. For example, the agent may decrease the expression of a member of the LDL receptor gene family in a cell normally expressing such a member of the LDL receptor gene family, or alternatively the agent may increase the expression of a member of the LDL receptor gene family in a cell. For example, MPR is known to reduce retinol and RBP levels in serum. Chronic treatment of mice with MPR any result in decreased expression of identified LDL receptor gene family proteins in the RPE that are responsible for transcytosis of RBP, thus establishing a relationship between LDL receptor gene family proteins in the RPE and serum RBP-retinol.

Other methods for altering the expression of a member of the LDL receptor gene family are known in the art. For example, a nucleic acid sequence can be used to alter the expression of a member of the LDL receptor gene family. See for example, US 2004/0198705, paragraphs [0176] through [0186], and WO 2005/070965.

Furthermore, the agent may have the potential to alter the expression of a co-receptor of a member of the LDL receptor gene family in a cell. For example, the agent may decrease the expression of a co-receptor of a member of a LDL receptor gene family in a cell normally expressing such a member of the LDL receptor gene family or alternatively the agent may increase the expression of a co-receptor of a member of the LDL receptor gene family in a cell.

The agent contemplated herein can be selected from a library of naturally occurring and synthetic compounds, which are randomly tested for alteration of the binding.

### Polypeptides and Proteins

In one embodiment, the agent is a polypeptide. For example, such polypeptides could be selected from the group consisting of RBP binding protein receptor domains and fragments thereof, RBP binding protein co-receptor domains and fragments thereof, endogenous ligands that bind to members of the LDL receptor gene, modified retinoid binding proteins or fragments thereof, fragments of retinoid binding proteins, LDL receptor gene family antagonists, such as receptor associated protein (RAP), and functional homologues of any of these.

In one embodiment, the agent is a domain of a member of the LDL receptor gene family that can bind to a retinoid binding protein. Preferably, said domain of a member of the LDL receptor gene family is capable of binding a retinoid binding protein, such as RBP or IRBP. In one embodiment, the domain is a megalin domain. In one embodiment, the domain is a LRP domain.

It has been shown that complement type repeats of LRP are capable of binding a protein designated RAP. More specifically, everyone of the 8 complement type repeats of cluster 11 of LRP are able to bind RAP with the exception of repeat 8, which differs from the rest in that it lacks a negatively charged amino acid (Andersen et al., 2000, J. Biol. Chem. 275:21017-21024).

Accordingly, it is preferred that the domain of the member of the LDL receptor gene family includes at least one complement type repeat, more preferably, at least two complement type repeats. Other domains of the LDL receptor gene family are contemplated, such as a domain of the LDL receptor gene family that includes, for example, 2 complement type repeats, 3 complement type repeats, 4 complement type repeats, 5 complement type repeats, 6 complement type repeats, 7 complement type repeats, 8 complement type repeats, 9 complement type repeats, 10 complement type repeats, 11 complement type repeats, or more than 11 complement type repeats. In a preferred embodiment the domain of the member of the LDL receptor gene family includes 2 complement-type repeats. For specific domains of members of the LDL receptor gene family, see, for example, US 2004/0198705, paragraphs [0143] through [0164].

In another embodiment, the polypeptide is a fragment of a retinoid binding protein. Preferably, such a fragment is capable of associating with a member of the LDL receptor gene family in retina and RPE cells. Furthermore, such a fragment of a retinoid binding protein is not capable of binding or associating with a retinoid, such as, for example, retinol. In such a case, the fragment of a retinoid binding protein can bind the member of the LDL receptor gene family in retina and/or RPE cells and thereby inhibit binding of a retinol-RBP complex or a retinol-RBP-TTR complex with said member of the LDL receptor gene family.

In one embodiment, the agent is a fragment of RAP that can associate with a member of the LDL receptor gene family in retina and/or RPE cells that can bind retinoid binding proteins.

In another embodiment, the agent is an endogenous ligand to any of the members of the LDL receptor gene family. Members of the LDL receptor gene family are known to share common endogenous ligands (see above). Examples of endogenous ligands to members of the LDL receptor gene family, such as LRP and megalin, are presented above.

In one embodiment, the polypeptide is a light chain (Klassen et al. Light Chains are a Ligand for Megalin. J. Appl. Physiol. 98:257-263, 2005).

In one embodiment, the polypeptide is an antagonist to a member of the LDL receptor gene family in retina and/or RPE cells. Polypeptides can be screened for their ability to modulate the activity of members of the LDL receptor gene family in retina and/or RPE cells. In one embodiment, the polypeptides are screened for their ability to inhibit retinoid uptake into RPE cells. In another embodiment, the polypeptides are screened for their ability to inhibit IRBP-retinol and/or RBP-retinol uptake into RPE cells. In another embodiment, the polypeptides are screened for their ability to inhibit therapuetic drug uptake into retina and/or RPE cells. In another embodiment, the polypeptides are screened for their ability to inhibit antibiotic drug, such as, for example, aminoglyside drug, uptake into retina and/or RPE cells.

### Nucleic Acids

In one embodiment, the agent is a nucleic acid sequence. Preferably, such a nucleic acid sequence potentially alters the expression of a member of the LDL receptor gene family in retina and/or RPE cells. In one embodiment, the member of the LDL receptor gene family is a retinoid binding protein receptor.

In one embodiment, a nucleic acid sequence includes a DNA sequence encoding for an anti-sense RNA or a small interfering RNA (siRNA) of a member of the LDL receptor gene family that is present in retina and RPE cells or the nucleic acid sequence is an antisense RNA of a member of the LDL receptor gene family in retina and RPE cells. Homologues thereof are also within the scope of the present disclosure. In some embodiments, the member of the LDL receptor gene family is a retinoid binding protein.

Furthermore, the nucleic acid sequence may include an-antigene nucleic acid sequence, which is capable of hybridising with a gene encoding a member of the LDL receptor gene family in retina and/or RPE cells and thereby inhibiting transcription of said gene. Said antigene nucleic acid sequence may be capable of hybridising to any part of said gene, for example to the promotor and/or to introns and/or to exons of said gene. The antigene nucleic acid may be any kind of nucleic acid, for example DNA, RNA, LNA or PNA or siRNA.

As used herein, the term “antisense RNA” is intended to encompass an RNA sequence transcribed from the non-coding DNA strand of a member of the LDL receptor gene family in retina and/or RPE cells or an RNA sequence that is capable of hybridising to a member of the LDL receptor gene family mRNA under stringent conditions or fragments thereof.

If the nucleic acid sequence is a DNA sequence encoding an antisense RNA of a member of the LDL receptor gene family in retina and/or RPE cells or homologues thereof, such a nucleotide sequence is preferably operably linked to nucleotide sequences that directs transcription of said DNA sequence in the cell of the particular embodiment disclosed herein.

In another embodiment the nucleic acid sequence includes sequences encoding a member of the LDL receptor gene family in retina and/or RPE cells or homologues thereof or fragments thereof. Such a nucleic acid sequence is preferably operably linked to nucleotide sequences that directs transcription of said DNA sequence in the cell of the particular embodiment of the invention.

A variety of nucleotide sequences that directs transcription of DNA sequences are known to the person skilled in the art and such sequences should be selected according to the specific need in the individual case. For example such sequences could be promoter sequences and enhancer sequences of prokaryotic, eukaryotic or viral origin or they could be synthetic sequences.

The nucleic acid sequence may be included within a vector and any suitable vector known to the person skilled in the art may be employed. A vector is capable of delivering the nucleic acid molecule into a host cell. Such a vector contains nucleic acid sequences that are not naturally found adjacent to the nucleic acid sequences of the member of the LDL receptor gene family inretina and/or RPE cells.

A vector is a plasmid that can be used to transfer DNA sequences from one organism to another. A vector is a replicable construct which could be any nucleic acid including DNA, RNA, LNA and PNA. Once transformed into a suitable host, the vector replicates and functions independently of the host genome, or may, in some instances, integrate into the genome itself.

Typically the vector is a viral derived vector, a retroviral derived vector, a phage, a plasmid, a cosmid, an integratable DNA fragment (i.e., integratable into the host genome by recombination), bacteria or eukaryotic cells.

### Low Molecular Weight Organic Compounds

In some embodiments, the agent that modulates the activity of the member of the LDL receptor gene family in retina and/or RPE cells is a low molecular weight organic compound. Is some embodiments, the low molecular weight organic compound has a positive charge. In some embodiments, the low molecular weight organic compound has more than one positive charge. In some embodiments, the low molecular weight organic compound has 2 positive charges. In some embodiments, the low molecular weight organic compound has 3 positive charges. In some embodiments, the low molecular weight organic compound has 4 positive charges. In some embodiments, the low molecular weight organic compound has 5 positive charges. In some embodiments, the low molecular weight organic compound has 1, 2, 3, 4, or more than 4 positive charges. By selecting a low molecular weight organic compound with positive charges, it is possible to block a sufficient number of binding sites on the receptor protein. It is known that the binding sites in members of the LDL receptor gene family contain anionic amino acid residues that are capable of interacting with cationic species (see above).

In some embodiments, the low molecular weight compounds provided herein have an amino group. In some embodiments, the low molecular weight compound has two amino groups. In some embodiments, the low molecular weight compound has more than one amino group. In some embodiments, the low molecular weight compound has a functionality (group) that can accept a proton. In some embodiments, the low molecular weight compound has more than one functionality (group) that can accept a proton. In some embodiments, the low molecular weight compound has more than one functionality (group) that can accept more than one proton. Suitable functionalities that can accept a proton are amino groups.

In some embodiments, the low molecular weight organic compound has the structure of Formula (I):

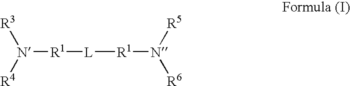

wherein, L is a bond, aryl, heteroaryl containing 0-3 N atoms, C₃-C₈ carbocycloalkyl, C₃-C₈ heterocycloalkyl containing 0-3 N atoms, wherein the aryl, heteroaryl, carbocycloalkyl or heterocycloalkyl is optionally substituted with O (oxo), OH, phenyl, halide, C₁-C₄ alkyl, C₂-C₄ alkenyl, C₂-C₄ alkynyl, heteroaryl, aryl-(C₁-C₄ alkyl), heteroaryl-(C₁-C₄ alkyl), heterocycloalkyl-(C₁-C₄ alkyl), cycloalkylalkyl, O—(C₁-C₄ alkyl), O(COR¹⁰), —CO₂H, —CO₂R¹⁰, —C(O)R¹⁰, —CON(R¹⁰)₂, —NHC(O)R¹⁰, —C(OH)(R¹⁰)₂, tetrazolyl, —C(O)NHSO₂R¹⁰, —CHOHCF₃, —COCF₃, —SO₂NHC(O)R¹⁰, or —N(R¹⁰)₂, where each R¹⁰ is independently H, or an optionally substituted group selected from lower alkyl, lower fluoroalkyl, lower alkenyl, lower alkynyl, C₃-C₆ cycloalkyl, phenyl, or benzyl; R¹ and R² are each independently selected from a bond and C₁-C₁₀ alkyl, wherein the C₁-C₁₀ alkyl is optionally substituted at least once with a substituent selected from among 0, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁-C₄)-alkyl, heteroaryl-(C₁-C₄)-alkyl, heterocyclyl-(C₁-C₄)-alkyl, cycloalkylalkyl, cycloalkyl, alkoxy, carboxy, trifluoromethyl, cyano, amino, or nitro, wherein any of the carbons in said C₁-C₁₀ alkyl is optionally replaced by oxygen, nitrogen, sulphur, or silicon; R³, R⁴, R⁵, and R⁶ individually are selected from among hydrogen, OH, trifluoromethyl, C(NH)NH₂, cyano, amino, nitro, an optionally substituted group selected from among alkyl, heteroalkyl, alkenyl, alkynyl, phenyl, benzyl, aryl, heterocycloalkyl, heteroaryl, aryl-(C₁-C₄)-alkyl, heteroaryl-(C₁-C₄)-alkyl, heterocyclyl-(C₁-C₄)-alkyl, cycloalkylalkyl, cycloalkyl, wherein the groups that are optionally substituted have a substituent selected from among H, O (oxo), OH, phenyl, imine(NH), halogen, C₁-C₄alkyl, C₂-C₄alkenyl or C₂-C₄alkynyl, aryl, heterocyclyl, heteroaryl, aryl-(C₁-C₄)-alkyl, heteroaryl-(C₁-C₄)-alkyl, heterocyclyl-(C₁-C₄)-alkyl, cycloalkylalkyl, cycloalkyl, alkoxy, carboxy, trifluoromethyl, cyano, amino, and or nitro; or
one or more of R³, R⁴, R⁵, and R⁶ is a bond to L; or
one or more of R³, R⁴, R⁵, and R⁶ is a linked to another R¹, R², R³, R⁴, R⁵, R⁶ and/or to L, thereby forming a ring;
wherein N′ and N″ optionally have a further group attached thus forming a quaternary ammonium salt; and
pharmaceutically acceptable salts, pharmaceutically acceptable N-oxides, pharmaceutically active metabolites, pharmaceutically acceptable prodrugs, and pharmaceutically acceptable solvates thereof.

In some embodiments, N′ and N″ are separated by at least 4 atoms.

In some embodiments, L is selected from among cyclopentyl, furan, thienyl, pyrrole, imidazole, oxazole, pyrrolidine, tetrahydrofuran, and tetahydrothiophene. In some embodiments, L is furan or pyrrole. In some embodiments, L is tetrahydrofuran. In some embodiments, L is selected from among pyridine, pyrimidine, tetrahydropyran, piperidine, piperazine, cyclohexyl, and phenyl. In some embodiments, L is cyclohexyl or phenyl. In some embodiments, L is a bond.

In some embodiments, the low molecular weight organic compound has the structure of Formula (II):

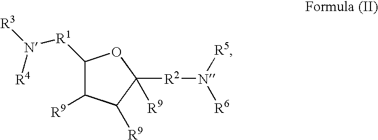

wherein:
each R⁹ is independently selected from among H, OH, O—(C₁-C₄ alkyl), O(COR¹⁰), halide, C₁-C₄ alkyl, (C₁-C₄ alkyl)-amino, —N(R¹⁰)₂ and aryl; and
the other variables are as herein described.

In some embodiments, the low molecular weigh organic compound has the structure of Formula (III):

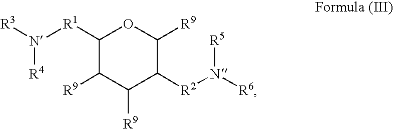

wherein:
each R⁹ is independently selected from among H, OH, O—(C₁-C₄ alkyl), O(COR¹⁰), halide, C₁-C₄ alkyl, (C₁-C₄ alkyl)-amino, —N(R¹⁰)₂ and aryl; and
the other variables are as herein described.

In some embodiments, the low molecular weight organic compound has the structure of Formula (IV):

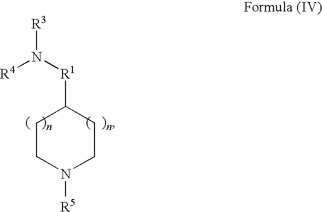

wherein:
each n is independently 0, 1, 2, or 3; and
the variables are as described herein above.

In some embodiments, the low molecular weight organic compound has the structure of Formula (V):

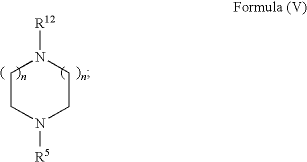

wherein:

### R¹² is H or

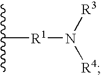

each n is independently 0, 1, 2, or 3; and
the other variables are as herein described.

In some embodiments, each n is 1.

In some embodiment, the low molecular weight organic compound is selected from among 1,2,-diaminoethane; 1,3-diaminopropane; 1,4-diaminobutane; 1,5-diaminopentane; 1,6-diaminohexame; 1,7-diaminoheptane; 1,8-diaminooctane; 3-methylamino-1-(4-methylpiperazino)-propan-2-ol; 4-piperazinoaniline; 1-(3-chlorophenyl)piperazine dihydrochloride; piperazin-2-one HCl; 2-[4-(2-aminoethyl)-piperazin-1-yl]-ethylamine; piperazine; 2,4-diamino-6-phenyl-1,3,5-triazine; 3,5-diamino-1,2,4-triazole; melonamide; arginine HCl; piperidine; 2,5-piperazinedione; piperazine anhydrous; piperazin-2-one HCl; and 1-(2-pyrimidyl)piperazine dihydrochloride; and pharmaceutically acceptable salts thereof.

In some embodiments, the low molecular weight organic compound is selected from among 2-[4-(2-aminoethyl)piperazin-1-yl]-ethylamine; 3-methylamino-1-(4-methylpiperazino)-propan-2-ol; and piperazine.

In some embodiments, the low molecular weight organic compound is piperazine.

In some embodiments, the low molecular weight organic compound is selected from among 1,2-diaminoethane; 1,3-diaminopropane; 1,4-diaminobutane; 1,5-diaminopentane; 1,6-diaminohexane; 1,7-diaminoheptane; and 1,8-diaminooctane.

In some embodiments, the low molecular weight organic compound is selected from among 1,2-diaminoethane; 1,4-diaminobutane; 1,5-diaminopentane; 1,6-diaminohexane; and 1,7-diaminoheptane.

In some embodiments, the low molecular weight organic compound is 1,6-diaminohexane.

In one embodiment, the agent is selected from among 4,4′-diaminodicyclohexylmethane

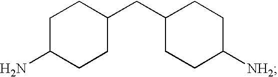

trans-1,4-diaminocyclohexane

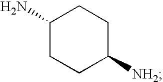

1,3-bis(aminomethyl)-cyclohexane

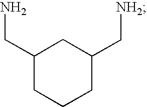

1,4-bis(aminomethyl)-cyclohexane

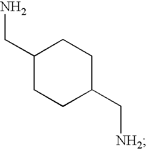

p-Xylylene diamine

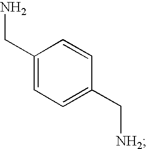

Xylylene diamine

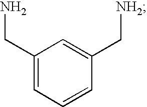

1-(pyrid-4-yl)-piperazine

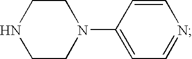

1-[4-((piperidin-1
yl)methyl)benzyl]piperidine

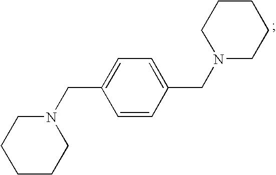

2,3,5,6-tetramethyl-1,4-xylylenediamine, dihydrochloride

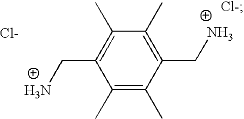

2,5-dimethyl-1,4-xylylenediamine, dihydrochloride

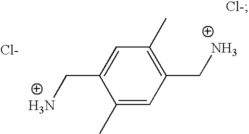

α,α′-(dimethylamino)-p-xylene, dihydrobromide

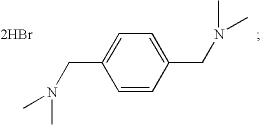

α,α′-(trimethylammonium)-2,5-dimethyl-p-xylene, dichloride

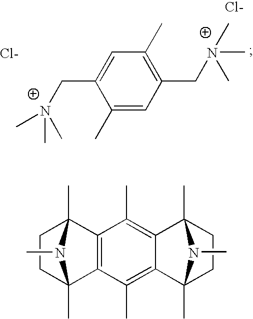

### (Sigma/Aldrich S111333); N-(4-Guanidinomethyl-benzyl)-guanidine

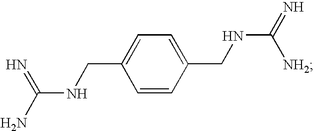

3-aminomethylbenzamidine, dihydrochloride

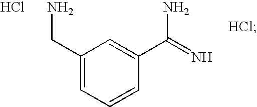

4-(aminomethyl)benzamidine dihydrochloride

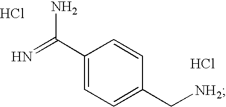

4-Aminomethyl-2,3,5,6-tetrachloro-benzylamine, dihydrochloride

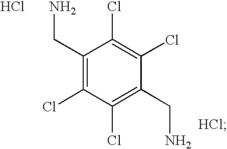

4-Aminomethyl-2,3,5,6-tetrafluoro-benzylamine, dihydrochloride

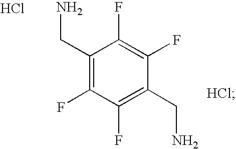

2,6-Diallyl-1,2,3,5,6,7-hexahydropyrrolo[3,4f]isoindole, dihydrochloride

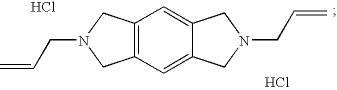

2-(4-Aminomethyl-phenyl)-ethylamine, dihydrochloride

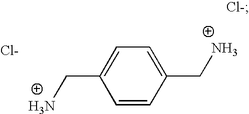

1,4-(Diamidino)benzene, dihydrochloride

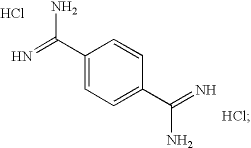

2-(4-(2-aminoethyl)phenyl)ethanamine, dihydrochloride

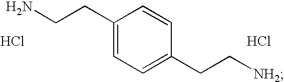

3-(4-methylpiperazin-1-yl)propan-1-amine

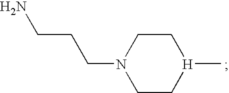

2-amino-1-(4-(aminomethyl)phenyl)ethanol, dihydrochloride

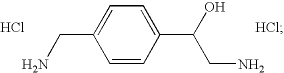

1,4-di(2-amino-1-hydroxyethyl)benzene, dihydrochloride

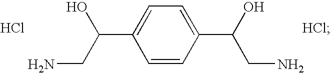

2-(4-methylpiperazin-1-yl)ethanamine, trihydrochloride

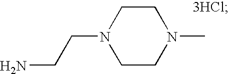

4-(4-methylpiperazin-1-yl)butan-1-amine, trihydrochloride

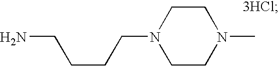

2-(piperazin-1-yl)ethanamine, trihydrochloride

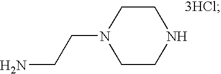

3-(piperazin-1-yl)propan-1-amine, trihydrochloride

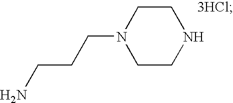

3-(4,4-dimethylpiperazin-1-yl)-propan-1-amine

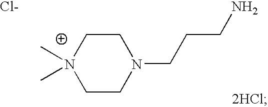

1 (2-aminoethyl)piperidin-4-amine, trihydrochloride

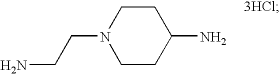

1-(3-aminopropyl)piperidin-4-amine, trihydrochloride

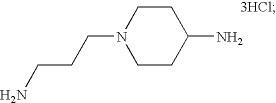

2-(piperidin-4-yl)ethanamine, dihydrochloride

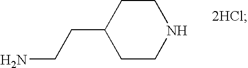

3-(piperidin-4-yl)propan-1-amine, dihydrochloride

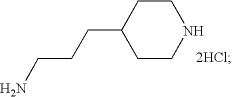

(2R,3S,4S,5R)-2,5-bis(aminomethyl)-tetrahydrofuran-3,4-diol, dihydrochloride

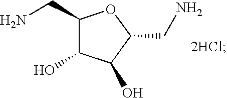

(2R,3S,4S,5R)-2,5-bis(guanidinomethyl)-tetrahydrofuran-3,4-diol, dihydrochloride

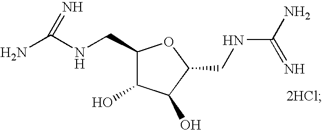

(2R,3S,4S,5R)-2,5-bis(2-aminoethyl)-tetrahydrofuran-3,4-diol, dihydrochloride

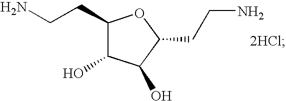

(2R,3S,4S,5R)-2,5-bis(2-guanidinoethyl)tetrahydrofuran-3,4-diol, dihydrochloride

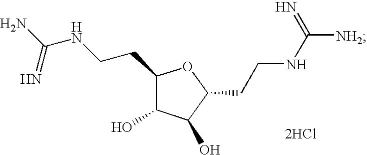

4-(piperidin-4-yl)piperidine dihydrochloride

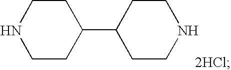

(2R,3S,4S,5R)-2-(2-aminoethyl)-5-(aminomethyl)-tetrahydrothran-3,4-diol dihydrochloride

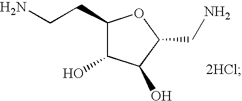

(2S,3S,4R,5S,6S)-5-amino-2-(aminomethyl)-6-methoxy-tetrahydro-2H-pyran-3,4-diol, dihydrochloride

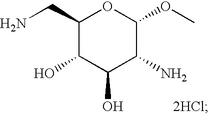

(2R,3S,4R,5R,6S)-5-amino-2-(2-aminoethyl)-6-methoxy-tetrahydro-2H-pyran-3,4-diol, dihydrochloride

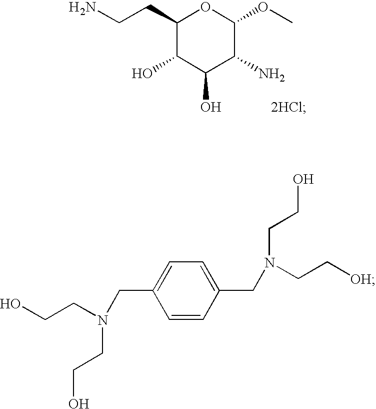

(2R,3R,4R,5R)-1,6-diaminohexane-2,3,4,5-tetraol, dihydrochloride

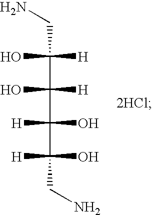

(2S,3R,4R,5R)-1,6-diaminohexane-2,3,4,5-tetraol, dihydrochloride

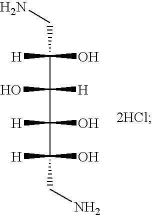

and (2S,3R,4S,5R)-1,6-diaminohexane-2,3,4,5-tetraol, dihydrochloride

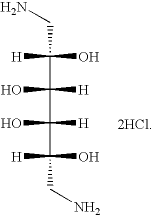

In another embodiment, the agent is selected from among 4,4′-diaminodicyclohexylmethane

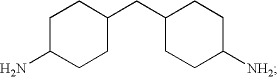

trans-1,4-diaminocyclohexane

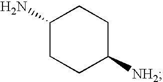

1,3-bis(aminomethyl)-cyclohexane

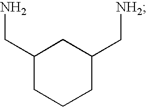

1,4-bis(aminomethyl)-cyclohexane

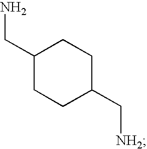

p-Xylylene diamine

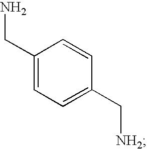

m-Xylylene diamine

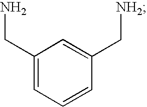

1-[4-((piperidin-1-yl)methyl)benzyl]piperidine

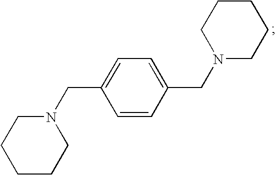

2,3,5,6-tetramethyl-1,4-xylylenediamine, dihydrochloride

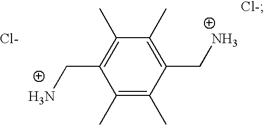

2,5-dimethyl-1,4-xylylenediamine, dihydrochloride

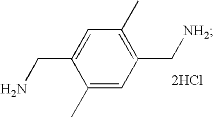

α,α′-(dimethylamino)-p-xylene, dihydrobromide

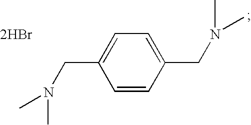

α,α′-(trimethylammonium)-2,5-dimethyl-p-xylene, dichloride

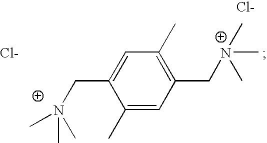

### N-(4-Guanidinomethyl-benzyl)-guanidine

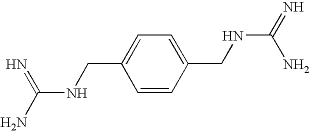

3-aminomethylbenzamidine, dihydrochloride

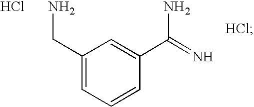

4-(aminomethyl)benzamidine dihydrochloride

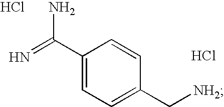

4-Aminomethyl-2,3,5,6-tetrachloro-benzylamine, dihydrochloride

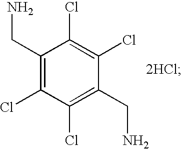

4-Aminomethyl-2,3,5,6-tetrafluoro-benzylamine, dihydrochloride

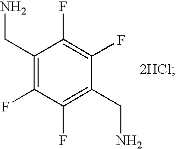

2-(4-Aminomethyl-phenyl)-ethylamine, dihydrochloride

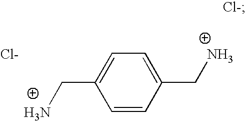

1,4-(Diamidino)benzene, dihydrochloride

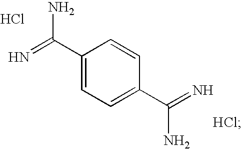

2-(4-(2-aminoethyl)phenyl)ethanamine, dihydrochloride

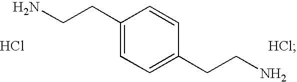

2-amino-1-(4-(aminomethyl)phenyl)ethanol, dihydrochloride

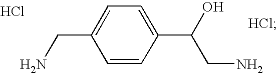

1,4-di(2-amino-1-hydroxyethyl)benzene, dihydrochloride

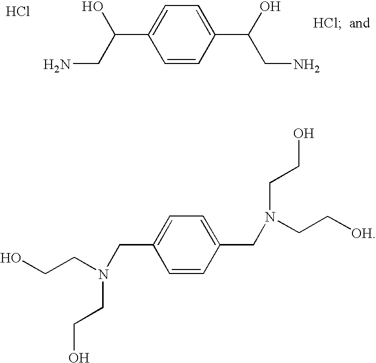

In one embodiment, the agent is selected from among trans-1,4-diaminocyclohexane

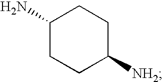

1,3-bis(aminomethyl)-cyclohexane

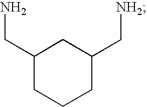

1,4-bis(aminomethyl)-cyclohexane

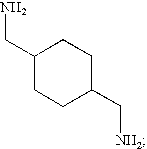

p-Xylylene diamine

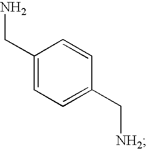

m-Xylylene diamine

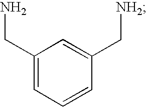

2,5-dimethyl-1,4-xylylenediamine, dihydrochloride

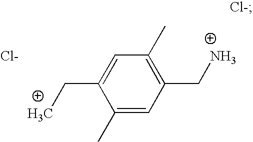

α,α′-(dimethylamino)-p-xylene, dihydrobromide

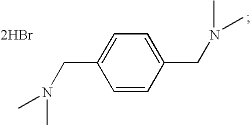

α,α′-(trimethylammonium)-2,5-dimethyl-p-xylene, dichloride

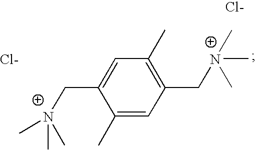

### N-(4-Guanidinomethyl-benzyl)-guanidine

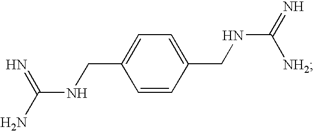

3-aminomethylbenzamidine, dihydrochloride

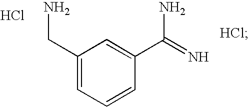

4-(aminomethyl)benzamidine dihydrochloride

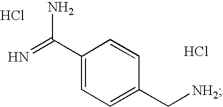

2-(4-Aminomethyl-phenyl)-ethylamine, dihydrochloride

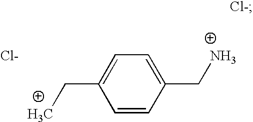

2-(4-(2-aminoethyl)phenyl)ethanamine, dihydrochloride

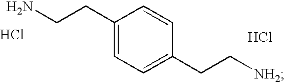

and 1,4-di(2-amino-1-hydroxyethyl)benzene, dihydrochloride

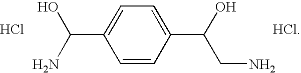

In one embodiment, the agent is selected from among (2R,3S,4S,5R)-2,5-bis(aminomethyl)-tetrahydrofuran-3,4-diol, dihydrochloride

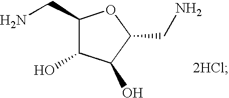

(2R,3S,4S,5R)-2,5-bis(guanidinomethyl)-tetrahydrofuran-3,4-diol, dihydrochloride

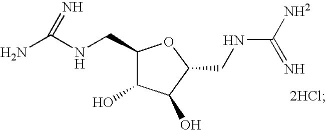

(2R,3S,4S,5R)-2,5-bis(2-aminoethyl)-tetrahydrofuran-3,4-diol, dihydrochloride

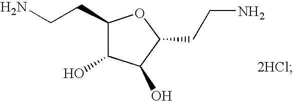

(2R,3S,4S,5R)-2,5-bis(2-guanidinoethyl)tetrahydrofuran-3,4-diol, dihydrochloride

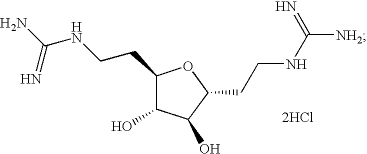

(2R,3S,4S,5R)-2-(2-aminoethyl)-5-(aminomethyl)-tetrahydrothran-3,4-diol dihydrochloride

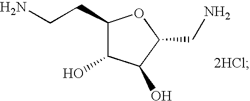

(2S,3S,4R,5S,6S)-5-amino-2-(aminomethyl)-6-methoxy-tetrahydro-2H-pyran-3,4-diol, dihydrochloride

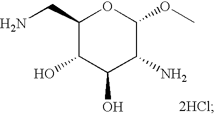

and (2R,3S,4R,5R,6S)-5-amino-2-(2-aminoethyl)-6-methoxy-tetrahydro-2H-pyran-3,4-diol, dihydrochloride

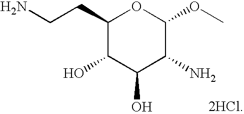

In one embodiment, the agent is (2R,3S,4S,5R)-2,5-bis(aminomethyl)-tetrahydrofuran-3,4-diol, dihydrochloride

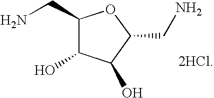

In one embodiment, the agent is selected from among

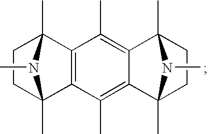

and 2,6-Diallyl-1,2,3,5,6,7-hexahydropyrrolo[3,4-f]isoindole, dihydrochloride

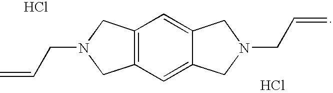

In one embodiment, the agent is selected from among 1-(pyrid-4-yl)-piperazine

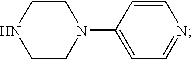

3-(4-methylpiperazin-1-yl)propan-1-amine

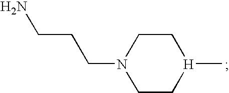

2-(4-methylpiperazin-1-yl)ethanamine, trihydrochloride

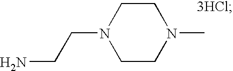

4-(4-methylpiperazin-1-yl)butan-1-amine, trihydrochloride

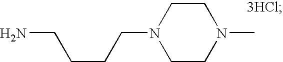

2-(piperazin-1-yl)ethanamine, trihydrochloride

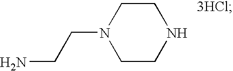

3-(piperazin-1-yl)propan-1-amine, trihydrochloride

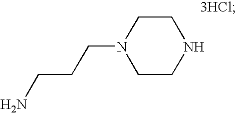

3-(4,4-dimethylpiperazin-1-yl)-propan-1-amine

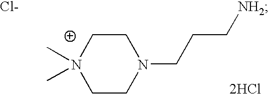

1-(2-aminoethyl)piperidin-4-amine, trihydrochloride

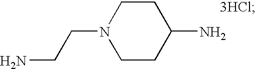

1-(3-aminopropyl)piperidin-4-amine, trihydrochloride

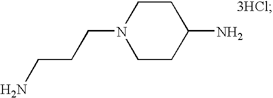

2-(piperidin-4-yl)ethanamine, dihydrochloride

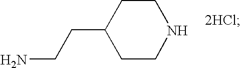

3-(piperidin-4-yl)propan-1-amine, dihydrochloride

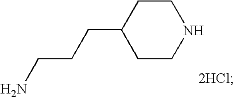

and 4-(piperidin-4-yl)piperidine dihydrochloride

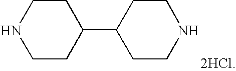

In one embodiment, the agent is selected from among 3-(4-methylpiperazin-1-yl)propan-1-amine

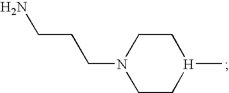

4-(4-methylpiperazin-1-yl)butan-1-amine, trihydrochloride

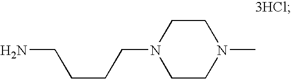

3-(4,4-dimethylpiperazin-1-yl)-propan-1-amine

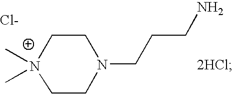

2-(piperidin-4-yl)ethanamine, dihydrochloride

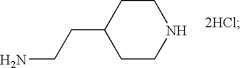

and 4-(piperidin-4-yl)piperidine dihydro chloride

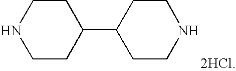

In one embodiment, the agent is selected from among (2R,3R,4R,5R)-1,6-diaminohexane-2,3,4,5-tetraol, dihydrochloride

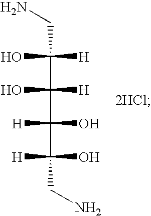

(2S,3R,4R,5R)-1,6-diaminohexane-2,3,4,5-tetraol, dihydrochloride

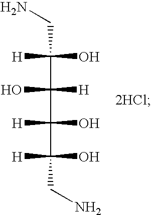

and (2S,3R,4S,5R)-1,6-diaminohexane-2,3,4,5-tetraol, dihydrochloride

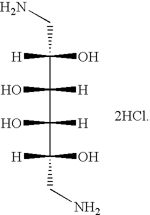

In another embodiment, the agent is selected from among trans 1,4-diaminocyclohexane; 1,3-bis(aminomethyl)-cyclohexane; 1,4-bis(aminomethyl)-cyclohexane; p-xylylene diamine; m-xylylene diamine; 1-(4-(pyrid-4-yl)-piperazine; 2,5-dimethyl-1,4-xylylene-diamine dihydrochloride; α,α′-(dimethylamino)-p-xylene dihydrobromide; and

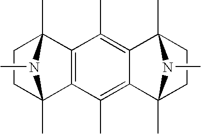

### (Sigma/Aldrich S111333).

In one embodiment, the compound is selected from among trans 1,4-diaminocyclohexane; 1,3-bis(aminomethyl)-cyclohexane; 1,4-bis(aminomethyl)-cyclohexane; p-xylylene diamine; m-xylylene diamine; 2,5-dimethyl-1,4-xylylene-diamine dihydrochloride; α,α′-(dimethylamino)-p-xylene dihydrobromide.

In another embodiment, the compound is selected from among trans 1,4-diaminocyclohexane; p-xylylene diamine; m-xylylene diamine; 2,5-dimethyl-1,4-xylylene-diamine dihydrochloride; 1-(pyrid-4-yl)-piperazine, α,α′-(dimethylamino)-p-xylene dihydrobromide.

In another embodiment, the compound is selected from among p-xylylene diamine; 1-(pyrid-4-yl)-piperazine, α,α′-(dimethylamino)-p-xylene dihydrobromide.

In another embodiment, the compound is selected from among trans p-xylylene diamine and α,α′-(dimethylamino)-p-xylene dihydrobromide.

In one embodiment, the low molecular weight organic compounds inhibits the uptake of retinoids into RPE cells. In another embodiment, the low molecular weight organic compounds inhibits the uptake of RBP-retinol and/or IRBP-retinol into RPE cells. In another embodiment, the low molecular weight organic compounds inhibits the uptake of retinal-toxic theraputic drugs into RPE cells.

### Derivatives of Aminoglycosides

In another embodiment, an agent that modulates the activity of a member of the LDL receptor gene family in retina and/or RPE cells is a derivative of aminoglycosides. Aminoglycosides have been shown to bind to members of the LDL receptor gene family. See for example, WO 2004/084876. Derivatives of aminoglycosides have been shown to have potential as antagonists of members of the LDL receptor gene family. See for, example, WO 2004/084876. In one embodiment, the agent is a derivative of gentamicin, Polymyxin B, Aprotinin, Trichosanthin, amikacin, kanamycin, neomycin, netilmicin, paromomycin, streptomycin, tobramycin or apramycin. In one embodiment, the agent is a derivative of gentamicin, Polymyxin B, Aprotinin, or Trichosanthin. In another embodiment, the agent is a derivative of gentamicin. In another embodiment, the agent is a derivative of gentamicin selected from among garoseamine, purposamine, and 2-deoxystreptamine.

### Ca²⁺ Scavengers

Members of the LDL receptor family are known to bind Ca²⁺, which is thought to contribute to receptor stability and maintain the receptor in its the native conformation, which is crucial for the binding of certain ligands to the receptor (Andersen et al. J. Biol. Chem. Vol 275, no. 28, 21017-21024, 2000). Certain protein ligands, such as, for example, RBP, IRBP and RAP bind to the receptor protein in its native conformation. In some embodiments, the agent is a Ca²⁺ scavenger used to modulate the activity of the member of the LDL receptor gene family in retina and/or RPE cells. In some embodiments, a Ca²⁺ scavenger decreases the stability of the receptor protein. In some embodiments, a Ca²⁺ scavenger is EDTA. In some embodiments, the Ca²⁺ scavenger is added with a second agent.

### Disulfide Reducing Agents

The Ligand-binding (complement) type cysteine-rich repeats that are present in the members of the LDL receptor gene family contain multiple disulfide bridges that contribute to the three-dimensional structure of the receptor protein (Andersen et al. J. Biol. Chem. Vol 275, no. 28, 21017-21024, 2000). Certain protein ligands, such as, for example, RBP and IRBP, recognize and bind to members of the LDL receptor gene family only when the receptor protein is in its native form. Reduction of the disulfide bridges disrupts the native conformation of the receptor proteins, and significantly inhibits the binding of protein ligands (see, for example, US 2003/0202974). In one embodiment, the agent is a reducing agent. In another embodiment, the agent reduces the disulfide groups in the receptor protein.

### Polymers

In some embodiments, the agent is a polymer. In some embodiments, the polymer has at least one positive charge. In some embodiments, the polymer has more than one positive charge. In one embodiment, the polymer is polylysine. In another embodiment, the polymer is a derivative of polylysine. Other polymers contemplated herein include those disclosed in WO 2004/084876 and WO 2006/037335. Polymers of any of the peptides or proteins disclosed herein are also contemplated.

### Antibodies

In some embodiments, the agent used herein to modulate the activity of a member of the LDL receptor gene family in retina and/or RPE cells is an antibody. Described herein are antibodies, and methods of making antibodies, that specifically recognize a member of the LDL receptor gene family in retina and/or RPE cells. Antibodies are obtained through commercial vendors, such as, for example Fitzgerald Industries International, Inc. (Concord, Mass.), Santa Cruz Technologies (Santa Cruz, Calif.), Oxford Biomedical Research (Oxford, Mich.). Alternatively, antibodies specific to members of the LDL receptor gene family in retina and/or RPE cells are obtained by methods known in the art.

The immunogen for producing an appropriate antibody can include the complete member of the LDL receptor gene family that is expressed in retina and/or RPE cells, or fragments and derivatives thereof, or members of the LDL receptor gene family that are expressed on the surface of retina and/or RPE cells. Immunogens include all or a part of one of the member of the LDL receptor gene family, where these amino acids contain post-translational modifications, such as glycosylation, found on the native target protein. Immunogens including protein extracellular domains are produced in a variety of ways known in the art, e.g., expression of cloned genes using conventional recombinant methods, or isolation from tumor cell culture supernatants, etc. The immunogen can also be expressed in vivo from a polynucleotide encoding the immunogenic peptide introduced into the host animal.

Antibody molecules contemplated herein include immunoglobulin molecules, which are typically composed of heavy and light chains, each of which have constant regions that display similarity with other immunoglobulin molecules and variable regions that convey specificity to particular antigens. Most immunoglobulins can be assigned to classes, e.g., IgG, IgM, IgA, IgE, and IgD, based on antigenic determinants in the heavy chain constant region; each class plays a different role in the immune response.

Antibodies can be used to modulate biological activity, either by increasing or decreasing a stimulation, inhibition, or blockage in the measured activity when compared to a suitable control.

Antibody modulators include antibodies that specifically bind a member of the LDL receptor gene family in retina and/RPE cells and activate the receptor protein, such as receptor-ligand binding that initiates signal transduction; antibodies that specifically bind a member of the LDL receptor gene family and inhibit binding of another molecule to the polypeptide, thus preventing activation of a signal transduction pathway; antibodies that bind a member of the LDL receptor gene family to modulate transcription; and antibodies that bind a member of the LDL receptor gene family to modulate translation. An antibody that modulates a biological activity of a member of the LDL receptor gene family, or polynucleotide thereof, increases or decreases the activity or binding at least about 10%, at least about 15%, at least about 20%, at least about 25%, at least about 50%, at least about 100%, or at least about 2-fold, at least about 5-fold, or at least about 10-fold or more when compared to a suitable control. In one embodiment, an antibody specifically interferes with the activity of a member of the LDL receptor gene family in retina and/or RPE cells. More specifically, the antibody specifically binds to the extracellular domain of a member of the LDL receptor gene family in retina and/or RPE cells.

In one embodiment, the agent is an intrabody. The intrabodies are intracellularly expressed single-chain antibody molecules designed to specifically bind and inactivate target molecules inside cells. Intrabodies have been used in cell assays and in whole organisms (Chen et al., Hum. Gene Ther. 5:595 (1994); Hassanzadeh et al., FEBS Lett. 437:75 (1998). Inducible expression vectors can be constructed with intrabodies that react specifically with a protein receptor that belongs to the LDL receptor gene family that is expressed in retina and/or RPE cells. These vectors can be introduced into host cells and model organisms.

In one embodiment, the agent is an “artificial” antibodies, e.g., antibodies and antibody fragments produced and selected in vitro. In some embodiments, these antibodies are displayed on the surface of a bacteriophage or other viral particle, as described above. In other embodiments, artificial antibodies are present as fusion proteins with a viral or bacteriophage structural protein, including, but not limited to, M13 gene III protein. Methods of producing such artificial antibodies are well known in the art (U.S. Pat. Nos. 5,516,637; 5,223,409; 5,658,727; 5,667,988; 5,498,538; 5,403,484; 5,571,698; and 5,625,033). The artificial antibodies, selected for example, on the basis of phage binding to selected antigens, can be fused to a Fc fragment of an immunoglobulin for use as a therapeutic, as described, for example, in U.S. Pat. No. 5,116,964 or WO 99/61630. Antibodies can be used to modulate biological activity of cells, either directly or indirectly. A subject antibody can modulate the activity of a target cell, with which it has primary interaction, or it can modulate the activity of other cells by exerting secondary effects, i.e., when the primary targets interact or communicate with other cells. The antibodies provided herein can be administered to mammals, particularly for therapeutic and/or diagnostic purposes in humans.

In one embodiment, the agent is an antibody. In another embodiment, the antibody is a human or humanized antibody. In another embodiment, the antibody is a polyclonal antibody, monoclonal antibody, single chain antibody, agonist antibody, an antagonist antibody, a neutralizing antibody, or active fragments thereof. In one embodiment, the active fragment of an antibody is a fragment that specifically binds to an antigen or an epitope. In one embodiment, the active fragment is an antigen-binding fragment, a Fc fragment, a cdr fragment, a VH fragment, a VL fragment or a framework fragment. In one embodiment, the antibody includes at least one domain selected from a variable region of an immunoglobulin, a constant region of an immunoglobulin, a heavy chain of an immunoglobulin, a light chain of an immunoglobulin and an antigen-binding region of an immunoglobulin. In one embodiment, the antibody includes at least one light chain of an immunoglobulin.

### Peptide Aptamers

Another suitable agent for modulating an activity of a member of the LDL receptor gene family in retina and/or RPE cells is a peptide aptamer. Peptide aptamers are peptides or small polypeptides that act as dominant inhibitors of protein function. Peptide aptamers specifically bind to target proteins, blocking their functional ability (Kolonin et al., Proc. Natl. Acad. Sci. USA 95:14266 (1998). Due to the highly selective nature of peptide aptamers, they can be used not only to target a specific protein, but also to target specific functions of a given protein (e.g., a signaling function). Further, peptide aptamers can be expressed in a controlled fashion by use of promoters that regulate expression in a temporal, spatial or inducible manner. Peptide aptamers act dominantly, therefore, they can be used to analyze proteins for which loss-of-function mutants are not available.

Peptide aptamers that bind with high affinity and specificity to a target protein can be isolated by a variety of techniques known in the art. Peptide aptamers can be isolated from random peptide libraries by yeast two-hybrid screens (Xu et al., Proc. Natl. Acad. Sci. USA 94:12473 (1997). They can also be isolated from phage libraries (Hoogenboom et al., Immunotechnology 4:1 (1998) or chemically generated peptides/libraries.

### Endogenous Binding Ligands to the Members of the LDL Receptor Gene Family

As described above, the term “endogenous binding ligand” is meant to include an endogenous primary substance that binds to a member of the LDL receptor gene family, including megalin and megalin-like proteins, as well as a secondary endogenous substance that binds to the primary binding ligand of the member of the LDL receptor gene family when the primary binding substance is bound to the member of the LDL receptor gene family. Those with skill in the art will appreciate based upon the present description, that the particular endogenous binding ligand detected and measured will depend upon a number of factors, including, for example, the ability of the ligand to be readily detectable if taken up into retina and/or RPE cells. A variety of megalin binding ligands are known to exist, including, for example, those listed above (see also, Chistensen, I. L. and Willnow, T. E. J. Am. Soc. Nephrol. 10, 2224-2236, 1999). Preferred endogenous megalin binding ligand as presented herein include retinoid binding protein and interphotoreceptor retinoid binding protein. Additional endogenous megalin binding ligands may be identified by one or more of the methods described in Christensen et al. (1992), Chistensen, I. L. and Willnow, T. E. (1999) J. Am. Soc. Nephrol. 10, 2224-2236; Cui, S. et al. (1996) Am. J. Physiol. 271, F900-F907; Gburek, J. et al. (2002) J. Am. Soc. Nephrol. 13, 423-430; Hilpert et al. (1999), J. Biol. Chem. 274, 5620-5625; Kanalas, J. J. and Makker, S. P. (1991) J. Biol. Chem. 266, 10825-10829; Kounnas, M. Z. et al. (1992) J. Biol. Chem. 267, 21162-21166; Kounnas, M. Z. et al. (1993) J. Biol. Chem. 268, 14176-14181; Kounnas, M. Z. et al (1995) J. Biol. Chem. 270, 13070-13075; Moestrup, S. K. et al. (1993) J. Biol. Chem. 268, 16564-16570; Moestrup, S. K. et al. (1995) J. Clin. Invest. 96, 1404-1413; Moestrup, S. K. et al. (1996) Proc. Natl. Acad. Sci. USA 93, 8612-8617; Moestrup, S. K. et al. (1998) J. Clin. Invest. 102, 902-909; Nykjaer, A. et al. (1999) Cell 96, 507-515; Orlando, R. A. et al. (1992) Proc. Natl. Acad. Sci. USA 89, 6698-6702; Orlando, R. A. et al. (1998) J. Am. Soc. Nephrol. 9, 1759-1766; Stefansson, S. et al. (1995-A) J. Cell Sci. 108, 2361-2368; Stefansson, S. et al. (1995-B) J. Biol. Chem. 270, 19417-19421; Wilnow, T. E. et al. (1992) J. Biol. Chem. 267, 26172-26180; Wilnow, T. E. et al. (1996) Proc. Natl. Acad. Sci. USA 93, 8460-8464; and Zheng, G. et al. (1998) Endocrinology 139, 1462-1465.

It will be appreciated by those with skill in the art based upon the present description, that the method of detection and quantification of endogenous megalin binding ligands may include any of a number of available analytical tools. For example, such methods may include the use of HPLC, NMR, or by using standard immunoassay methods known in the art. Such immunoassays include, but are not limited to, competitive and non-competitive assay systems using techniques such as RIAs, ELISAs, “sandwich” immunoassays, immunoradiometric assays, gel diffusion precipitin reactions, immunodiffusion assays, in situ immunoassays (using, for example, colloidal gold, enzymatic, or radioisotope labels), Western blots, 2-dimensional gel analysis, precipitation reactions, immunofluorescence assays, protein A assays, and immunoelectrophoresis assays.

### Identification of Ligand Binding Regions

The complement-type ligand-binding repeats within the LDL receptor gene family are responsible for recognition of ligands. (Herz et al., LRP:a multifunctional scavenger and signaling receptor. The Journal of Clinical Investigation, vol 108, no. 6, pp 779-784, 2001). Ligand recognition properties of the LDL receptor gene family identified herein can be accomplished by methods known in the art. Briefly, and by way of example only, regions responsible for binding a number of ligands may be accomplished using the following methods. “Minireceptors” of the identified receptors can be prepared by fusing various clusters of ligand binding repeats to the membrane spanning and cytoplasmic domains of the receptor and measuring their ability to mediate the cellular internalization of ligands following expression in cells. (Willnow et al., Molecular dissection of ligand binding sites on the low density lipoportein receptor related protein. J. Biol. Chem. 269:15827-15832, 1994). Another approach may involve testing soluble recombinant receptor fragments representing each of the clusters in the receptor for the ability to bind various ligands in vitro (Springer, T A. An extracellular beta-propellar module predicted in lipoprotein and scavenger receptors, tyrosine kinases, epidermal growth factor precursor, and extra cellular matrix components. J. Mol. Biol. 283:837-862, 1998).

Binding of numerous structurally distinct ligands with high affinity arises from the presence of multiple ligand-binding-type repeats in the receptors, from the unique contour surface and charge distribution for each repeat, and from multiple interactions between both the ligand and the receptor. Some ligands can recognize different repeats in a sequential fashion, while others appear to recognize repeats from separate clusters. (Herz et al., LRP:a multifunctional scavenger and signaling receptor. The Journal of Clinical Investigation, vol 108, no. 6, pp 779-784, 2001).

### Assays

Methods of determining whether agents bind to and/or modulate the activity of members of the LDL receptor gene family in retina and/or RPE cells are known in the art. For example, assays described in the art include those outlined in: US 2003/0202974, WO 06/037335, WO 03/080103, US 2004/0198705, WO 04/084876, US 2006/0029609, US 2005/0026823, US 2005/0100986, US 2005/0089932, US 2005/0042227, US 2004/0204357, US 2004/0198705, US 2004/0049010, US 2003/0202974, US 2003/0181660, US 2003/0082640, US 2003/0157561, US 2003/0077672, Chistensen, et al. (1999) J. Am. Soc. Nephrol. 10, 2224-2236; Cui, S. et al. (1996) Am. J. Physiol. 271, F900-F907; Gburek, J. et al. (2002) J. Am. Soc. Nephrol. 13, 423-430; Hilpert et al. (1999), J. Biol. Chem. 274, 5620-5625; Kanalas, J. J. and Malker, S. P. (1991) J. Biol. Chem. 266, 10825-10829; Kounnas, M. Z. et al. (1992) J. Biol. Chem. 267, 21162-21166; Kounnas, M. Z. et al. (1993) J. Biol. Chem. 268, 14176-14181; Kounnas, M. Z. et al (1995) J. Biol. Chem. 270, 13070-13075; Moestrup, S. K. et al. (1993) J. Biol. Chem. 268, 16564-16570; Moestrup, S. K. et al. (1995) J. Clin. Invest. 96, 1404-1413; Moestrup, S. K. et al. (1996) Proc. Natl. Acad. Sci. USA 93, 8612-8617; Moestrup, S. K. et al. (1998) J. Clin. Invest. 102, 902-909; Nykjaer, A. et al. (1999) Cell 96, 507-515; Orlando, R. A. et al. (1992) Proc. Natl. Acad. Sci. USA 89, 6698-6702; Orlando, R. A. et al. (1998) J. Am. Soc. Nephrol. 9, 1759-1766; Stefansson, S. et al. (1995-A) J. Cell Sci. 108, 2361-2368; Stefansson, S. et al. (1995-B) J. Biol. Chem. 270, 19417-19421; Wilnow, T. E. et al. (1992) J. Biol. Chem. 267, 26172-26180; Wilnow, T. E. et al. (1996) Proc. Natl. Acad. Sci. USA 93, 8460-8464; and Zheng, G. et al. (1998) Endocrinology 139, 1462-1465.

Identification of an agent that interacts with (i.e. binds and/or modulates the activity of) member of the LDL receptor gene family in the retina and/or RPE cells can be detected using any known method. Suitable methods include: a yeast two-hybrid system (Zhu et al., Proc. Natl. Acad. Sci. USA 94:13,063 (1997); Fields et al., Nature 340:245 (1989); U.S. Pat. No. 5,283,173; Chien et al., Proc. Natl. Acad. Sci. USA 88:9578 (1991); a mammalian cell two-hybrid method; a fluorescence resonance energy transfer (FRET) assay; a bioluminescence resonance energy transfer (BRET) assay; a fluorescence quenching assay; a fluorescence anisotropy assay (Jameson et al., Methods Enzymol. 246:283 (1995); an immunological assay; and an assay involving binding of a detectably labeled protein to an immobilized protein.

Methods of detecting the presence and biological activity of members of the LDL receptor gene family in a biological sample are known. The assays used will be appropriate to the biological activity of the particular member of the LDL receptor gene family. Thus, e.g., where the biological activity is binding to a second macromolecule, the assay detects protein-protein binding, protein-DNA binding, protein-carbohydrate binding, or protein-lipid binding, as appropriate, using well known assays. Where the biological activity is signal transduction (e.g., transmission of a signal from outside the cell to inside the cell) or transport, an appropriate assay is used, such as measurement of intracellular calcium ion concentration, measurement of membrane conductance changes, or measurement of intracellular potassium ion concentration.

Provided herein are methods for detecting the presence or measuring the level of normal or abnormal retinoid binding protein receptors that belong to the LDL receptor gene family in a biological sample using a specific antibody. The methods generally include contacting the sample with a specific antibody and detecting binding between the antibody and molecules of the sample. Specific antibody binding, when compared to a suitable control, is an indication that a member of the LDL receptor gene family of interest is present in the sample.

A variety of methods to detect specific antibody-antigen interactions are known in the art, e.g., standard immunohistological methods, immunoprecipitation, enzyme immunoassay, and radioimmunoassay. Briefly, antibodies are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, specific-binding pairs may be used, involving, e.g., a second stage antibody or reagent that is detectably-labeled, as described above. Such reagents and their methods of use are well known in the art

Methods of identifying agents that modulate a biological activity of a member of the LDL receptor gene family are known. The methods generally include contacting a test agent with a sample containing the subject polypeptide, such as a member of the LDL receptor gene family, and assaying a biological activity of the subject member of the LDL receptor gene family in the presence of the test agent. An increase or a decrease in the assayed biological activity in comparison to the activity in a suitable control (e.g., a sample comprising a subject member of the LDL receptor gene family in the absence of the test agent) is an indication that the substance modulates a biological activity of the subject polypeptide. The mixture of components is added in any order that provides for the requisite interaction.

Methods for identifying an agent, particularly a biologically active agent that modulates the level of expression of nucleic acid of a member of the LDL receptor gene family in cells are known. The method includes: combining a candidate agent to be tested with a cell comprising a nucleic acid that encodes the member of the LDL receptor gene family, and determining the agent's effect on expression of the member of the LDL receptor gene family.

Agents that decrease a biological activity of a member of the LDL receptor gene family in retina and/or RPE cells can find use in treating conditions or disorders associated with the biological activity of the molecule. For example, regulation of the expression of members of the LDL receptor gene family in retina and/or RPE cells can be used to treat ophthalmic disorders. A decreased level of expression of members of the LDL receptor gene family in retina and/or RPE cells can reduce the amount of retinol, retinol-RBP, and/or retinol-RBP-TTR that is taken up in the retina and RPE cells that normally express the members of the LDL receptor gene family. A decreased level of expression of members of the LDL receptor gene family in retina and/or RPE cells can reduce the amount of therapeutic drug (i.e., those which produce undesired ocular-toxic side effects) that is taken up into retina and/or RPE cells that normally express the members of the LDL receptor gene family. A decreased level of expression of members of the LDL receptor gene family in retina and/or RPE cells can reduce the amount of antibiotics, such as, for example, aminoglycosides, that is taken up in the retina and RPE cells that normally express the members of the LDL receptor gene family.

Alternatively, some embodiments will detect agents that increase a biological activity.

In one embodiment, RPE cells are treated with RBP-retinol and/or IRBP-retinol and an agent presented herein. After a period of time, the cells are isolated and assayed for retinol content (including RBP and IRBP content). The amount of retinol that is found within the RPE cells as compared to a control (RPE cells that are treated with RBP-retinol and/or IRBP-retinol and without an agent presented herein) will provide an indication of the effect of the agent on receptor mediated activity (i.e. inhibition of RBP-retinol or IRBP-retinol receptor-mediated transcytosis).

In another embodiment, RPE cells are treated with a therapuetic drug, such as, for example, an antibiotic drug and an agent presented herein. The therapuetic drug will contribute to toxic effects in the ocular tissues, such as, for example, retina and/or RPE cells. After a period of time, the cells are isolated and assayed for therapuetic drug content. The amount of therapuetic drug that is found within the retina and/or RPE cells as compared to a control (retina and/or RPE cells that are treated with the therapuetic drug, without an agent presented herein) will provide an indication of the effect of the agent on receptor mediated activity (i.e. inhibition of therapuetic drug receptor-mediated transcytosis).

Agents that increase a biological activity of a member of the LDL receptor gene family in retina and/or RPE cells can find use in treating ophthalmic conditions associated with a deficiency in the biological activity. For example, increased biological activity of a member of the LDL receptor gene family and lead to increased transcytosis in retina and/or RPE cells and thus either increase retinoid concentrations in said cells or prevent accumulation of retinoids and/or toxic chemicals in said cells.

A variety of different candidate agents can be screened by the above methods. Candidate agents encompass numerous chemical classes, as described above.

Candidate agents are obtained from a wide variety of sources including libraries of synthetic or natural compounds. Numerous means are available for random and directed synthesis of a wide variety of organic compounds and biomolecules, including expression of randomized oligonucleotides and oligopeptides. For example, random peptide libraries obtained by yeast two-hybrid screens (Xu et al, Proc. Natl. Acad. Sci. USA 94:12473 (1997), phage libraries (Hoogenboom et al., Immunotechnology 4:1 (1998), or chemically generated libraries.

Alternatively, libraries of natural compounds in the form of bacterial, fungal, plant and animal extracts are available or readily produced, including antibodies produced upon immunization of an animal with subject polypeptides, or fragments thereof, or with the encoding polynucleotides. Additionally, natural or synthetically produced libraries and compounds are readily modified through conventional chemical, physical and biochemical means, and can be used to produce combinatorial libraries. Further, known pharmacological agents can be subjected to directed or random chemical modifications, such as acylation, alkylation, esterification, and amidification, etc, to produce structural analogs.

In one embodiment, a method for evaluating whether an agent is modulating the activity of a member of the LDL receptor gene family is carried out with an animal model.

### Pharmaceutical Compositions

Another aspect are pharmaceutical compositions comprising an agent that modulates the activity of a member of the LDL receptor gene family in retina and/or RPE cells, such as, for example, Megalin-modulating agents, and a pharmaceutically acceptable diluent, excipient, or carrier.

Another aspect are pharmaceutical compositions comprising a Megalin-modulating agent and a pharmaceutically acceptable diluent, excipient, or carrier.

The term “pharmaceutical composition” refers to a mixture of an agent that modulates the activity of a member of the LDL receptor gene family in retina and/or RPE cells, such as, for example, a Megalin-modulating agent, with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “carrier” refers to relatively nontoxic chemical compounds or agents that facilitate the incorporation of a compound into cells or tissues.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The term “physiologically acceptable” refers to a material, such as a carrier or diluent, that does not abrogate the biological activity or properties of the compound, and is nontoxic.

The term “pharmaceutically acceptable salf” refers to a formulation of a compound that does not cause significant irritation to an organism to which it is administered and does not abrogate the biological activity and properties of the compound. Pharmaceutically acceptable salts may be obtained by reacting an agent that modulates a member of the LDL receptor gene family in retina and/or RPE cells, such as, for example, a Megalin-modulating agent, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts may also be obtained by reacting an agent that modulates a member of the LDL receptor gene family in retina and/or RPE cells, such as, for example, a Megalin-modulating agent with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art

A “metabolite” of a compound disclosed herein is a derivative of that compound that is formed when the compound is metabolized. The term “active metabolite” refers to a biologically active derivative of a compound that is formed when the compound is metabolized. The term “metabolized” refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be an agent that modulates a member of the LDL receptor gene family in retina and/or RPE cells, such as, for example, a Megalin-modulating agent, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

The agents that modulate the activity of a member of the LDL receptor gene family in retina and/or RPE cells described herein can be administered to a human patient per se, or in pharmaceutical compositions where they are mixed with other active ingredients, as in combination therapy, or suitable carrier(s) or excipient(s). Techniques for formulation and administration of the compounds of the instant application may be found in “Remington: The Science and Practice of Pharmacy,” 20th ed. (2000).

### Routes of Administration

Suitable routes of administration may, for example, include oral, rectal, transmucosal, transdermal, pulmonary, ophthalmic or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into an organ, often in a depot or sustained release formulation. Furthermore, one may administer the drug in a targeted drug delivery system, for example, in a liposome coated with organ-specific antibody. The liposomes will be targeted to and taken up selectively by the organ. In addition, the drug may be provided in the form of a rapid release formulation, in the form of an extended release formulation, or in the form of an intermediate release formulation.

### Composition/Formulation

Pharmaceutical compositions comprising an agent that modulates a member of the LDL receptor gene family in retina and/or RPE cells, such as, for example, a Megalin-modulating agent, may be manufactured in a manner that is itself known, e.g., by means of conventional mixing, dissolving, granulating, dragee-making, levigating, emulsifying, encapsulating, entrapping or compression processes.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

The agents that modulate a member of the LDL receptor gene family in retina and/or RPE cells, such as, for example, Megalin-modulating agents, can be administered in a variety of ways, including all forms of local delivery to the eye. Additionally, the agents that modulate a member of the LDL receptor gene family in retina and/or RPE cells, such as, for example, Megalin-modulating agents, can be administered systemically, such as orally or intravenously. The agents that modulate a member of the LDL receptor gene family in retina and/or RPE cells, such as, for example, Megalin-modulating agents, can be administered topically to the eye and can be formulated into a variety of topically administrable ophthalmic compositions, such as solutions, suspensions, gels or ointments. Thus, “ophthalmic administration” encompasses, but is not limited to, intraocular injection, subretinal injection, intravitreal injection, periocular administration, subconjuctival injections, retrobulbar injections, intracameral injections (including into the anterior or vitreous chamber), sub-Tenon's injections or implants, ophthalmic solutions, ophthalmic suspensions, ophthalmic ointments, ocular implants and ocular inserts, intraocular solutions, use of iontophoresis, incorporation in surgical irrigating solutions, and packs (by way of example only, a saturated cotton pledget inserted in the formix).

Administration of a composition to the eye generally results in direct contact of the agents with the cornea, through which at least a portion of the administered agents pass. Often, the composition has an effective residence time in the eye of about 2 to about 24 hours, more typically about 4 to about 24 hours and most typically about 6 to about 24 hours.

A composition comprising an agent that modulates a member of the LDL receptor gene family in retina and/or RPE cells, such as, for example, a Megalin-modulating agent, can illustratively take the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition may include a gel formulation. In other embodiments, the liquid composition is aqueous. Alternatively, the composition can take the form of an ointment.

Useful compositions can be an aqueous solution, suspension or solution/suspension, which can be presented in the form of eye drops. A desired dosage can be administered via a known number of drops into the eye. For example, for a drop volume of 25 μl, administration of 1-6 drops will deliver 25-1501 of the composition. Aqueous compositions typically contain from about 0.01% to about 50%, more typically about 0.1% to about 20%, still more typically about 0.2% to about 10%, and most typically about 0.5% to about 5%, weight/volume of an agent that modulates the activity of a member of the LDL receptor gene family in retina and/or RPE cells, such as, for example, a Megalin-modulating agent.

Typically, aqueous compositions have ophthalmically acceptable pH and osmolality. “Ophthalmically acceptable” with respect to a formulation, composition or ingredient typically means having no persistent detrimental effect on the treated eye or the functioning thereof, or on the general health of the subject being treated. Transient effects such as minor irritation or a “stinging” sensation are common with topical ophthalmic administration of agents and consistent with the formulation, composition or ingredient in question being “ophthalmically acceptable.”

Useful aqueous suspension can also contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions can also comprise an ophthalmically acceptable mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful compositions may also include ophthalmically acceptable solubilizing agents to aid in the solubility of an agent that modulates a member of the LDL receptor gene family in retina and/or RPE cells, such as, for example, a Megalin-modulating agent. The term “solubilizing agent” generally includes agents that result in formation of a micellar solution or a true solution of the agent. Certain ophthalmically acceptable nonionic surfactants, for example polysorbate 80, can be useful as solubilizing agents, as can ophthalmically acceptable glycols, polyglycols, e.g., polyethylene glycol 400, and glycol ethers.

Useful compositions may also include one or more ophthalmically acceptable pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

Useful compositions may also include one or more ophthalmically acceptable salts in an amount required to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful compositions may also include one or more ophthalmically acceptable preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions may include one or more ophthalmically acceptable surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions may include one or more antioxidants to enhance chemical stability where required. Suitable antioxidants include, by way of example only, ascorbic acid and sodium metabisulfite.

Aqueous suspension compositions can be packaged in single-dose non-reclosable containers. Alternatively, multiple-dose reclosable containers can be used, in which case it is typical to include a preservative in the composition.

The ophthalmic composition may also take the form of a solid article that can be inserted between the eye and eyelid or in the conjunctival sac, where it releases the agent. Release is to the lacrimal fluid that bathes the surface of the cornea, or directly to the cornea itself, with which the solid article is generally in intimate contact. Solid articles suitable for implantation in the eye in such fashion are generally composed primarily of polymers and can be biodegradable or non-biodegradable.

For intravenous injections, the agents that modulate a member of the LDL receptor gene family in retina and/or RPE cells, such as, for example, Megalin-modulating agents, may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

For oral administration, the agents that modulate a member of the LDL receptor gene family in retina and/or RPE cells, such as, for example, Megalin-modulating agents, can be formulated readily by combining the active compounds with pharmaceutically acceptable carriers or excipients well known in the art. Such carriers enable the agents described herein to be formulated as tablets, powders, pills, dragees, capsules, liquids, gels, syrups, elixirs, slurries, suspensions and the like, for oral ingestion by a patient to be treated. Pharmaceutical preparations for oral use can be obtained by mixing one or more solid excipient with one or more of the agents described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner.

Another useful formulation for administration of agents that modulate a member of the LDL receptor gene family in retina and/or RPE cells, such as, for example, Megalin-modulating agents, employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252. Such patches may be constructed for continuous, pulsatile, or on demand delivery of pharmaceutical agents. Still further, transdermal delivery of the agents can be accomplished by means of iontophoretic patches and the like. Transdermal patches can provide controlled delivery of the compounds. The rate of absorption can be slowed by using rate-controlling membranes or by trapping the compound within a polymer matrix or gel. Conversely, absorption enhancers can be used to increase absorption. Formulations suitable for transdermal administration can be presented as discrete patches and can be lipophilic emulsions or buffered, aqueous solutions, dissolved and/or dispersed in a polymer or an adhesive. Transdermal patches may be placed over different portions of the patient's body, including over the eye.

Additional iontophoretic devices that can be used for ocular administration of agents that modulate a member of the LDL receptor gene family in retina and/or RPE cells, such as, for example, Megalin-modulating agents, are the Eyegate applicator, created and patented by Optis France S. A., and the Ocuphor™ Ocular iontophoresis system developed Iomed, Inc.

For administration by inhalation, the agents that modulate the activity of a member of the LDL receptor gene family in retina and/or RPE cells, such as, for example, Megalin-modulating agents, are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as rectal gels, rectal foam, rectal aerosols, suppositories or retention enemas, e.g., containing conventional suppository bases such as cocoa butter or other glycerides. The compounds may also be formulated in vaginal or urethral compositions, including vaginal or urethral suppositories (bougies), medicated tampons, and vaginal tablets.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Injectable depot forms may be made by forming microencapsulated matrices (also known as microencapsule matrices) of an agent that modulates a member of the LDL receptor gene family in retina and/or RPE cells, such as, for example, a Megalin-modulating agent, in biodegradable polymers. Depending upon the ratio of drug to polymer and the nature of the particular polymer employed, the rate of drug release can be controlled. Depot injectable formulations may be also prepared by entrapping the drug in liposomes or microemulsions. By way of example only, posterior juxtascleral depots may be used as a mode of administration for agents that modulate a member of the LDL receptor gene family in retina and/or RPE cells, such as, for example, Megalin-modulating agents. The sclera is a thin avascular layer, comprised of highly ordered collagen network surrounding most of vertebrate eye. Since the sclera is avascular it can be utilized as a natural storage depot from which injected material cannot rapidly removed or cleared from the eye. The formulation used for administration of the compound into the scleral layer of the eye can be any form suitable for application into the sclera by injection through a cannula with small diameter suitable for injection into the scleral layer. Examples for injectable application forms are solutions, suspensions or colloidal suspensions.

Alternatively, other delivery systems for hydrophobic pharmaceutical compounds may be employed. Liposomes and emulsions are well known examples of delivery vehicles or carriers for hydrophobic drugs. Certain organic solvents such as N-methylpyrrolidone also may be employed, although usually at the cost of greater toxicity. Additionally, the compounds may be delivered using a sustained-release system, such as semipermeable matrices of solid hydrophobic polymers containing therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of therapeutic reagent, additional strategies for protein stabilization may be employed.

All of the formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

Many of the agents may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

### Diagnostic Methods for Detection of Fluorescent Compounds

The early diagnosis of retinal diseases, such as, for example, macular degenerations and/or macular dystrophies is important in order to initiate prompt therapeutic interventions. Detecting and/or measuring the presence of fluorescent compounds in ocular tissues is provided in US patent publication 2006/0099714, incorporated by reference. Provided herein are techniques and methods for detection of toxic fluorescent compounds, such as, for example, oxidized phospholipids and oxidized fatty acids, in ocular tissues.

Phospholipids and fatty acids are abundantly found in retina and/or RPE cells and are essential for the proper functioning of RPE and retina cells. The presence of and accumulation of toxic compounds in retina and/or RPE cells provides the basis for ocular diseases, such as, for example, macular degenerations and/or macular dystrophies. The early detection of such toxic compounds in ocular tissues is important in order to initiate prompt therapeutic interventions. The presence of oxidized phospholipids and fatty acids in RPE and/or retina cells has been correlated to ocular disease. Phospholipids and fatty acids can undergo light-induced and/or chemical-induced oxidation in retina and/or RPE cells. Oxidized phospholipids and oxidized fatty acids are fluorescent compounds that are capable of being detected by fluorescence spectrometry. Methods for the detection of fluorescent compounds in ocular tissues is presented in US patent publication 2006/0099714, incorporated by reference.

Oxidized phospholipids and oxidized fatty acids have different fluorescent emission spectra than other retinal toxic compounds, such as, for example, N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, and N-retinylidene-phosphatidylethanolamine.

In one embodiment, provided herein is a method for the early diagnosis of retinal diseases, such as, for example, macular degenerations and/or macular dystrophies that includes measuring the presence and/or amount of oxidized phospholipids (including oxidized phosphatidyl serine) and oxidized fatty acids (including docosahexanoic acid) oxidized in ocular tissues.

In one embodiment, provided herein is a method for measuring the presence of oxidized phospholipids and/or fatty acids in a sample. In some embodiment, the presence of oxidized phospholipids and/or fatty acids in a sample is determined by illuminating the sample with light having a wavelength between 300 and 400 nm, and measuring the emission fluorescence from the sample between 400 and 500 nm.

Phospholipids and fatty acids are taken up by RPE cells slowly. However, oxidized phopholipids and oxidized fatty acids are taken up at a rate approximately 10 times that of unoxidized phospholipids and fatty acids. Oxidized phopholipids and oxidized fatty acids are taken by RPE cells by receptor mediated trancytosis. In one embodiment, oxidized phopholipids and oxidized fatty acids are taken by RPE cells by members of the LDL receptor gene family.

### Treatment Methods, Dosages and Combination Therapies

The term “mammal” means all mammals including humans. Mammals include, by way of example only, humans, non-human primates, cows, dogs, cats, goats, sheep, pigs, rats, mice and rabbits.

The term “effective amount” as used herein refers to that amount of the compound being administered which will relieve to some extent one or more of the symptoms of the disease, condition or disorder being treated.

The compositions containing the compound(s) described herein can be administered for prophylactic and/or therapeutic treatments. The term “treating” is used to refer to either prophylactic and/or therapeutic treatments. In therapeutic applications, the compositions are administered to a patient already suffering from a disease, condition or disorder, in an amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician. It is considered well within the skill of the art for one to determine such therapeutically effective amounts by routine experimentation (e.g., a dose escalation clinical trial).

In prophylactic applications, compositions containing the agents that modulate a member of the LDL receptor gene family in retina and/or RPE cells, such as, for example, Megalin-modulating agents, described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial).

The terms “enhance” or “enhancing” means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system. When used in a patient, amounts effective for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered simultaneously (or over a short period of time) or at appropriate intervals, for example as two, three, four or more sub-doses per day.

In certain instances, it may be appropriate to administer at least one of the agents that modulate a member of the LDL receptor gene family in retina and/or RPE cells, such as, for example, Megalin-modulating agents, described herein (or a pharmaceutically acceptable salt, ester, amide, prodrug, or solvate) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Or, by way of example only, therapeutic effectiveness of one of the an agent that modulates a member of the LDL receptor gene family in retina and/or RPE cells, such as, for example, Megalin-modulating agents, described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the agents that modulates a member of the LDL receptor gene family in retina and/or RPE cells, such as, for example, Megalin-modulating agents, described herein with another therapeutic agent (which also includes a therapeutic regimen) that also has therapeutic benefit. By way of example only, in a treatment for macular degeneration involving administration of one of the agents that modulates a member of the LDL receptor gene family in retina and/or RPE cells, such as, for example, Megalin-modulating agents, described herein, increased therapeutic benefit may result by also providing the patient with other therapeutic agents or therapies for macular degeneration. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of at least one an agent that modulates a member of the LDL receptor gene family in retina and/or RPE cells, such as, for example, Megalin-modulating agent, with nitric oxide (NO) inducers, statins, negatively charged phospholipids, anti-oxidants, minerals, anti-inflammatory agents, anti-angiogenic agents, matrix metalloproteinase inhibitors, carotenoids, 13-cis-retinoic acid, or a compound having the structure of Formula (A):

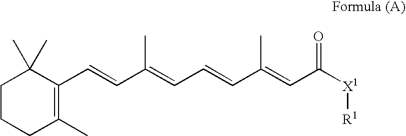

wherein

X₁ is selected from the group consisting of NR², O, S, CHR²;

R¹ is (CHR²)ₓ-L¹-R³, wherein
x is 0, 1, 2, or 3; L¹ is a single bond or —C(O)—;
R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and
R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle.

In several instances, suitable combination agents may fall within multiple categories (by way of example only, lutein is an anti-oxidant and a carotenoid). Further, the agent that modulates a member of the LDL receptor gene family in retina and/or RPE cells, such as, for example, Megalin-modulating agents, may also be administered with additional agents that may provide benefit to the patient, including by way of example only cyclosporin A.

In addition, the agents that modulate a member of the LDL receptor gene family in retina and/or RPE cells, such as, for example, Megalin-modulating agents, may also be used in combination with procedures that may provide additional or synergistic benefit to the patient, including, by way of example only, the use of extracorporeal rheopheresis (also known as membrane differential filtration), the use of implantable miniature telescopes, laser photocoagulation of drusen, and microstimulation therapy.

The use of anti-oxidants has been shown to benefit patients with macular degenerations and dystrophies. See, e.g., Arch. Opthalmol., 119: 1417-36 (2001); Sparrow, et al., J. Biol. Chem., 278:18207-13 (2003). Examples of suitable anti-oxidants that could be used in combination with an agent that modulates a member of the LDL receptor gene family in retina and/or RPE cells, such as, for example, a Megalin-modulating agent, include vitamin C, vitamin E, beta-carotene and other carotenoids, coenzyme Q, 4-hydroxy-2,2,6,6-tetramethylpiperidine-N-oxyl (also known as Tempol), lutein, butylated hydroxytoluene, resveratrol, a trolox analogue (PNU-83836-E), and bilberry extract.

The use of certain minerals has also been shown to benefit patients with macular degenerations and dystrophies. See, e.g., Arch. Opthalmol., 119: 1417-36 (2001). Examples of suitable minerals that could be used in combination with at least one an agent that modulates a member of the LDL receptor gene family in retina and/or RPE cells, such as, for example, Megalin-modulating agent, include copper-containing minerals, such as cupric oxide (by way of example only); zinc-containing minerals, such as zinc oxide (by way of example only); and selenium-containing compounds.

The use of certain negatively-charged phospholipids has also been shown to benefit patients with macular degenerations and dystrophies. See, e.g., Shaban & Richter, Biol. Chem., 383:537-45 (2002); Shaban, et al., Exp. Eye Res., 75:99-108 (2002). Examples of suitable negatively charged phospholipids that could be used in combination with at least one an agent that modulates a member of the LDL receptor gene family in retina and/or RPE cells, such as, for example, Megalin-modulating agent, include cardiolipin and phosphatidylglycerol. Positively-charged and/or neutral phospholipids may also provide benefit for patients with macular degenerations and dystrophies when used in combination with Megalin-modulating agents.

The use of certain carotenoids has been correlated with the maintenance of photoprotection necessary in photoreceptor cells. Carotenoids are naturally-occurring yellow to red pigments of the terpenoid group that can be found in plants, algae, bacteria, and certain animals, such as birds and shellfish. Carotenoids are a large class of molecules in which more than 600 naturally occurring carotenoids have been identified. Carotenoids include hydrocarbons (carotenes) and their oxygenated, alcoholic derivatives (xanthophylls). They include actinioerythrol, astaxanthin, canthaxanthin, capsanthin, capsorubin, β-8′-apo-carotenal (apo-carotenal), β-12′-apo-carotenal, α-carotene, β-carotene, “carotene” (a mixture of α- and β-carotenes), γ-carotenes, β-cyrptoxanthin, lutein, lycopene, violerythrin, zeaxanthin, and esters of hydroxyl- or carboxyl-containing members thereof. Many of the carotenoids occur in nature as cis- and trans-isomeric forms, while synthetic compounds are frequently racemic mixtures.

In humans, the retina selectively accumulates mainly two carotenoids: zeaxanthin and lutein. These two carotenoids are thought to aid in protecting the retina because they are powerful antioxidants and absorb blue light. Studies with quails establish that groups raised on carotenoid-deficient diets had retinas with low concentrations of zeaxanthin and suffered severe light damage, as evidenced by a very high number of apoptotic photoreceptor cells, while the group with high zeaxanthin concentrations had minimal damage. Examples of suitable carotenoids for in combination with at least one an agent that modulates a member of the LDL receptor gene family in retina and/or RPE cells, such as, for example, Megalin-modulating agent, include lutein and zeaxanthin, as well as any of the aforementioned carotenoids.

Suitable nitric oxide inducers include compounds that stimulate endogenous NO or elevate levels of endogenous endothelium-derived relaxing factor (EDRF) in vivo or are substrates for nitric oxide synthase. Such compounds include, for example, L-arginine, L-homoarginine, and N-hydroxy-L-arginine, including their nitrosated and nitrosylated analogs (e.g. nitrosated L-arginine, nitrosylated L-arginine, nitrosated N-hydroxy-L-arginine, nitrosylated N-hydroxy-L-arginine, nitrosated L-homoarginine and nitrosylated L-homoarginine), precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid) and the substrates for nitric oxide synthase, cytokines, adenosine, bradykinin, calreticulin, bisacodyl, and phenolphthalein. EDRF is a vascular relaxing factor secreted by the endothelium, and has been identified as nitric oxide or a closely related derivative thereof (Palmer et al, Nature, 327:524-526 (1987); Ignarro et al, Proc. Natl. Acad. Sci. USA, 84:9265-9269 (1987)).

Statins serve as lipid-lowering agents and/or suitable nitric oxide inducers. In addition, a relationship has been demonstrated between statin use and delayed onset or development of macular degeneration. G. McGwin, et al., British Journal of Opthalmology, 87:1121-25 (2003). Statins can thus provide benefit to a patient suffering from an ophthalnic condition (such as the macular degenerations and dystrophies, and the retinal dystrophies) when administered in combination with the Megalin-modulating agents. Suitable statins include, by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin.

Suitable anti-inflammatory agents with which the agents that modulate a member of the LDL receptor gene family in retina and/or RPE cells, such as, for example, Megalin-modulating agents, may be used include, by way of example only, aspirin and other salicylates, cromolyn, nedocromil, theophylline, zileuton, zafirlukast, montelukast, pranlukast, indomethacin, and lipoxygenase inhibitors; non-steroidal antiinflammatory drugs (NSAIDs) (such as ibuprofen and naproxin); prednisone, dexamethasone, cyclooxygenase inhibitors (i.e., COX-1 and/or COX-2 inhibitors such as Naproxen™, or Celebrex™); statins (by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin); and disassociated steroids.

Suitable matrix metalloproteinases (MMPs) inhibitors may also be administered in combination with the agents that modulate a member of the LDL receptor gene family in retina and/or RPE cells, such as, for example, Megalin-modulating agents, in order to treat ophthalmic conditions or symptoms associated with macular or retinal degenerations. MMPs are known to hydrolyze most components of the extracellular matrix. These proteinases play a central role in many biological processes such as normal tissue remodeling, embryogenesis, wound healing and angiogenesis. However, excessive expression of MMP has been observed in many disease states, including macular degeneration. Many MMPs have been identified, most of which are multidomain zinc endopeptidases. A number of metalloproteinase inhibitors are known (see for example the review of MMP inhibitors by Whittaker M. et al, Chemical Reviews 99(9):2735-2776 (1999)). Representative examples of MMP Inhibitors include Tissue Inhibitors of Metalloproteinases (TIMPs) (e.g., TIMP-1, TIMP-2, TIMP-3, or TIMP-4), α₂-macroglobulin, tetracyclines (e.g., tetracycline, minocycline, and doxycycline), hydroxamates (e.g., BATIMASTAT, MARIMISTAT and TROCADE), chelators (e.g., EDTA, cysteine, acetylcysteine, D-penicillamine, and gold salts), synthetic MMP fragments, succinyl mercaptopurines, phosphonamidates, and hydroxaminic acids. Examples of MMP inhibitors that may be used in combination with an agent that modulates a member of the LDL receptor gene family in retina and/or RPE cells, such as, for example, Megalin-modulating agents, include, by way of example only, any of the aforementioned inhibitors.

The use of antiangiogenic or anti-VEGF drugs has also been shown to provide benefit for patients with macular degenerations and dystrophies. Examples of suitable antiangiogenic or anti-VEGF drugs that could be used in combination with at least one an agent that modulates a member of the LDL receptor gene family in retina and/or RPE cells, such as, for example, Megalin-modulating agent, include Rhufab V2 (Lucentis™), Tryptophanyl-tRNA synthetase (TrpRS), Eye001 (Anti-VEGF Pegylated Aptamer), squalamine, Retaane™ 15 mg (anecortave acetate for depot suspension; Alcon, Inc.), Combretastatin A4 Prodrug (CA4P), Macugen™, Mifeprex™ (mifepristone-ru486), subtenon triamcinolone acetonide, intravitreal crystalline triamcinolone acetonide, Prinomastat (AG3340-synthetic matrix metalloproteinase inhibitor, Pfizer), fluocinolone acetonide (including fluocinolone intraocular implant, Bausch & Lomb/Control Delivery Systems), VEGFR inhibitors (Sugen), and VEGF-Trap (Regeneron/Aventis).

Other pharmaceutical therapies that have been used to relieve visual impairment can be used in combination with at least one agent that modulates a member of the LDL receptor gene family in retina and/or RPE cells, such as, for example, Megalin-modulating agent. Such treatments include but are not limited to agents such as Visudyne™ with use of a non-thermal laser, PKC 412, Endovion (NeuroSearch A/S), neurotrophic factors, including by way of example Glial Derived Neurotrophic Factor and Ciliary Neurotrophic Factor, diatazem, dorzolamide, Phototrop, 9-cis-retinal, eye medication (including Echo Therapy) including phospholine iodide or echothiophate or carbonic anhydrase inhibitors, AE-941 (AEterna Laboratories, Inc.), Sirna-027 (Sirna Therapeutics, Inc.), pegaptanib (NeXstar Pharmaceuticals/Gilead Sciences), neurotrophins (including, by way of example only, NT-4/5, Genentech), Cand5 (Acuity Pharmaceuticals), ranibizumab (Genentech), INS-37217 (Inspire Pharmaceuticals), integrin antagonists (including those from Jerini A G and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celltech Group plc), ATX-S10 (Hamamatsu Photonics), TGF-beta 2 (Genzyme/Celtrix), tyrosine kinase inhibitors (Allergan, SUGEN, Pfizer), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), and cyclosporin A. See U.S. Patent Application Publication No. 20040092435.

In any case, the multiple therapeutic agents (one of which is one of the agents that modulates a member of the LDL receptor gene family in retina and/or RPE cells, such as, for example, Megalin-modulating agents, described herein) may be administered in any order or even simultaneously. If simultaneously, the multiple therapeutic agents may be provided in a single, unified form, or in multiple forms (by way of example only, either as a single pill or as two separate pills). One of therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; we envision the use of multiple therapeutic combinations. By way of example only, a Megalin-modulating agent may be provided with at least one antioxidant and at least one negatively charged phospholipid; or an agent that modulates a member of the LDL receptor gene family in retina and/or RPE cells, such as, for example, a Megalin-modulating agent, may be provided with at least one antioxidant and at least one inducer of nitric oxide production; or an agent that modulates a member of the LDL receptor gene family in retina and/or RPE cells, such as, for example, a Megalin-modulating agent, may be provided with at least one inducer of nitric oxide productions and at least one negatively charged phospholipid; and so forth.

In addition, the agents that modulate a member of the LDL receptor gene family in retina and/or RPE cells, such as, for example, Megalin-modulating agents, may also be used in combination with procedures that may provide additional or synergistic benefit to the patient. Procedures known, proposed or considered to relieve visual impairment include but are not limited to ‘limited retinal translocation’, photodynamic therapy (including, by way of example only, receptor-targeted PDT, Bristol-Myers Squibb, Co.; porfimer sodium for injection with PDT; verteporfin, QLT Inc.; rostaporfin with PDT, Miravent Medical Technologies; talaporfin sodium with PDT, Nippon Petroleum; motexafin lutetium, Pharmacyclics, Inc.), antisense oligonucleotides (including, by way of example, products tested by Novagali Pharma SA and ISIS-13650, Isis Pharmaceuticals), laser photocoagulation, drusen lasering, macular hole surgery, macular translocation surgery, implantable miniature telescopes, Phi-Motion Angiography (also known as Micro-Laser Therapy and Feeder Vessel Treatment), Proton Beam Therapy, microstimulation therapy, Retinal Detachment and Vitreous Surgery, Scleral Buckle, Submacular Surgery, Transpupillary Thermotherapy, Photosystem I therapy, use of RNA interference (RNAi), extracorporeal rheopheresis (also known as membrane differential filtration and Rheotherapy), microchip implantation, stem cell therapy, gene replacement therapy, ribozyme gene therapy (including gene therapy for hypoxia response element, Oxford Biomedica; Lentipak, Genetix; PDEF gene therapy, GenVec), photoreceptor/retinal cells transplantation (including transplantable retinal epithelial cells, Diacrin, Inc.; retinal cell transplant, Cell Genesys, Inc.), and acupuncture.

Further combinations that may be used to benefit an individual include using genetic testing to determine whether that individual is a carrier of a mutant gene that is known to be correlated with certain ophthalmic conditions. By way of example only, defects in the human ABCA4 gene are thought to be associated with five distinct retinal phenotypes including Stargardt disease, cone-rod dystrophy, age-related macular degeneration and retinitis pigmentosa. In addition, an autosomal dominant form of Stargardt Disease is caused by mutations in the ELOV4 gene. See Karan, et al., Proc. Natl. Acad. Sci. (2005). Patients possessing any of these mutations are expected to find therapeutic and/or prophylactic benefit in the methods described herein.

### ILLUSTRATIVE EXAMPLES

RPE Cultures—Human RPE cells were collected from post-mortem tissue and grown in Eagle's minimum essential medium without calcium (EMEM; Sigma Chemical, St. Louis, Mo.) with additives until the resident cells proliferated, reached confluence and were released into the medium. These non-attached cells were collected and grown on Millicell chambers with polycarbonate filters (Millipore, Bedford, Mass., USA) coated with mouse laminin (Collaborative Research, Bedford, Mass.). The Millicell chambers were maintained in multiwell plates, which allowed the separation of apical and basal medium compartments.

Reagents—MEM media w/Earle's salts and glutamine were from Cellgro (Hemdon, Va.). Rabbit, anti-rat megalin was a gift from Dr. Michele Marino (University of Pisa, Italy). Rabbit, anti-human gp330 was obtained from Fitzgerald Industries International, Inc (Concord, Mass.). Rabbit pre-immune IgG was obtained from Santa Cruz Technologies (Santa Cruz, Calif.) and receptor-associated protein (RAP) was obtained from Oxford Biomedical Research (Oxford, Mich.). Human retinol binding protein (RBP) was expressed in E. coli. The protein was purfied by ion exchange and size exclusion chromatography following denaturation and re-folding in the presence of retinol (Sigma, St Louis, Mo.). Bovine interstitial retinoid binding protein (IRBP) was prepared from frozen bovine retinas. Briefly, frozen retinas were placed in an isotonic buffer and mildly agitated overnight at 4° C. Soluble proteins were removed from the homogenate by centrifugation and IRBP was purified from the supernatant by sequential ConA sepharose and ion exchange chromatography. SDS-PAGE was used to check RBP and IRBP.

Immunocytochemistry—For confocal microscopy, RPE cultures on filters were fixed in 4% paraformaldehyde, serially dehydrated in ethanol and embedded in Epon. In some cases, cells were permeabilized with methanol for 5 min at −20° C. after fixation. Sections were analyzed using a Leica laser scanning confocal microscope (TCS—SP2, Leica, Exton, Pa.). A series of 1 μm x-y (en face) sections were collected. Each individual x-y image of the stained RPE cell cultures represents a three-dimensional projection of the entire optical section (sum of all images in the stack). Microscopic panels were composed using AdobePhotoshop 5.5. Bar=40 μm. Antibodies used are described above. Secondary antibodies included goat anti-mouse and rabbit Alexa 488 and Alexa 594 (Molecular Probes, Eugene, Oreg.).

Megalin-mediated Uptake of Retinol Binding Protein and Interstitial Retinoid Binding Protein in Human RPE Cells—A megalin-specific antibody (0.2 μg/ml), or RAP (0.5 μg/ml), was added to either the basal or apical compartment of the RPE cell culture and the samples were incubated for 2 hours at 4° C. Either RBP-retinol (30 μM) or IRBP-retinol (10 μM) was added to the appropriate compartment and incubation was resumed at 37° C. for 1 hour. Following incubation, the media was removed from both compartments and decanted. The RPE cells were removed from the filter support and processed as described below.

Tissue Preparation and Extraction—Two-hundred and fifty microliters of PBS containing 5 mM EDTA (pH 7.2) was added to the apical compartment and used to agitate the cells away from the filter insert. The cell suspension was transferred to a 1.5 ml centrifuge tube and the cells were centrifuged at 14,000×g for 5 minutes. The supernatant was discarded and the cell pellet was washed with an additional 100 μl of PBS. Following a second centrifugation, the RPE cell pellet was suspended in 50 μl of ddH₂O. Cell membranes were then treated with 100 μl of MeOH and 10 μl of 1M NH₂OH. The samples were incubated at room temperature for 5 minutes. Retinoids were extracted into 300 μl of dichloromethane (CH₂Cl₂). Following mixing and centrifugation (14,000×g, 1 min) the upper (organic phase) was removed and decanted. The aqueous (lower) phase was re-extracted twice more with 300 μl aliquots of CH₂Cl₂. The organic phases were pooled and the solvent was taken to dryness under a steam of nitrogen gas. Sample residues were resuspended in 210 μl of hexane for analysis by HPLC.

HPLC—Retinoid extracts were analyzed with an Agilent 1100 series high-performance liquid chromatograph (HPLC) equipped with a photodiode array detector using a silica column (Agilent Zorbax Rx-Sil 4.6 mm×250 mm, Agilent, Palo Alto, Calif.) and a gradient of dioxane in n-hexane at a flow rate of 2 mL/min.

Preparation of Megalin-enriched Membranes from Whole Tissue—Sucrose density centrifugation was used to separate the membranous constituents from kidney, retina and RPE-eyecup (RPE plus choroid and sclera) samples. Following dissection, tissue samples were homogenized in a buffer containing 0.25 M sucrose (pH 7.5). The homogenates were centrifuged at 27,000×g for 20 min. The supernatant fraction was discarded and the resulting pellet (P1) was homogenized in a buffer containing 0.5% CHAPS. Centrifugation was repeated to generate an insoluble pellet, which was discarded, and a CHAPS-soluble protein fraction (CS). The CS fraction was used as the protein source for all immunoblot experiments. This fraction was also used in de-glysosylation studies (treatment with 1 unit endoglycosidase F per μg of protein) to examine the effect of carbohydrate removal on electrophoretic mobility. In some experiments, P1 was resuspended in 1% triton in order to optimally immunoprecipitate megalin-immunoreactive proteins.

Peptide Sequencing—Megalin-immunoreactive proteins were cut from a SDS-PAGE gel and placed in siliconized Eppendorf tubes. The gel samples were destained with 200 μl of destaining solution (Sigma). After destaining, the gel pieces were dried and 5 units of PNGase F (Sigma) was added followed by incubation at 37° C. for 30 minutes. Water was added to cover the gel pieces and incubation at 37° C. was resumed for 12-16 hours. The incubation solution was discarded and the gel pieces were washed with water and sonicated at room temperature. Gel pieces were taken to dryness under vacuum. Trypsin (0.4 μg, Sigma) was added and sample was incubated for 30 minutes at 37° C. Following incubation, 50 μl of the Trypsin Reaction Buffer (Sigma) was added to the gel sample and incubation at 37° C. was resumed for 12-16 hours. After the incubation, the reaction solution was decanted and 50 μl of the Peptide Extraction Solution (Sigma) was added to elute the peptides. Following incubation at 37° C. for 30 min and intermittent vigorous agitation, the peptide-containing solution was removed and combined with the decanted reaction solution. The sample volume was reduced to ˜10 μl by evaporation under vacuum. Samples prepared in this manner were analyzed by mass spectroscopy on a capillary liquid chromatograph coupled to an electrospray ionization mass spectrometer (ESI-LC/MS) as described below.

ESI-LC/MS—An Agilent 1100 series capillary liquid chromatograph was used for chromatography. Peptides were separated by reverse phase chromatography using a Zorbax 300SB-C18 column (0.5×250 mm). A gradient of acetonitrile, containing 0.2% acetic Acid and 0.005% heptafluorobutyric acid, was pumped through the column at 5 μl/min. Column temperature was maintained at 50° C. The column eluate was delivered to an in-line electrospray ionization mass spectrometer (LCQ Deca XP plus, Thermo, San Jose, Calif.). The ESI source was programmed with the following parameters: spray voltage=4.04 kV, capillary voltage=42.34 V, capillary temperature=275.20° C., tube lens=20 V. Helium fragmentation energy varied between 25-30% to optimally dissociate the peptide fragments.

Immunoblot Analyses—Protein samples which were used for immunoblot analyses were resuspended in SDS loading buffer. These samples were electrophoresed on 3-8% Tris-Acetate gels (Invitrogen, Carlsbad, Calif.), and then transferred to PVDF membrane. The membrane was blocked with 5% milk in 0.1% Tween 20 dissolved in Tris-buffered saline (TBST), and then incubated with appropriate primary antibodies at 4° C. for 12-16 hours. The antibodies used for western blot included rabbit anti-rat megalin polyclonal antibody (5 μg/ml), and rabbit, anti-human RAP polyclonal anti-serum (1:500 dilution). After four washes with TBST, the membrane was incubated with horseradish proxidase-conjugated goat anti-rabbit IgG (1:100,000 dilution). The membrane was washed four times, developed with ChemiGlow West Substrate (Alpha Innotech, San Leandro, Calif.) and then visualized by a luminescence imager (Fluor Chem from Alpha Innotech).

ABCA4 Knockout Mice. ABCA4 encodes rim protein (RmP), an ATP-binding cassette (ABC) transporter in the outer-segment discs of rod and cone photoreceptors. The transported substrate for RmP is unknown. Mice generated with a knockout mutation in the abca4 gene, see Weng et al., Cell, 98:13-23 (1999), are useful for the study of RmP function as well as for an in vivo screening of the effectiveness for candidate substances. These animals manifest the complex ocular phenotype: (i) slow photoreceptor degeneration, (ii) delayed recovery of rod sensitivity following light exposure, (iii) elevated atRAL and reduced atROL in photoreceptor outer-segments following a photobleach, (iv) constitutively elevated phosphatidylethanolamine (PE) in outer-segments, and (v) accumulation of lipofuscin in RPE cells. See Weng et al., Cell, 98:13-23 (1999).

Rates of photoreceptor degeneration can be monitored in treated and untreated wild-type and abca4⁻/⁻ mice by two techniques. One is the study of mice at different times by ERG analysis and is adopted from a clinical diagnostic procedure. See Weng et al., Cell, 98:13-23 (1999). An electrode is placed on the corneal surface of an anesthetized mouse and the electrical response to a light flash is recorded from the retina. Amplitude of the α-wave, which results from light-induced hyperpolarization of photoreceptors, is a sensitive indicator of photoreceptor degeneration. See Kedzierski et al., Invest. Opthalmol. Vis. Sci., 38:498-509 (1997). ERGs are done on live animals. The same mouse can therefore be analyzed repeatedly during a time-course study. The definitive technique for quantitating photoreceptor degeneration is histological analysis of retinal sections. The number of photoreceptors remaining in the retina at each time point will be determined by counting the rows of photoreceptor nuclei in the outer nuclear layer.

Tissue Extraction. Eye samples were thawed on ice in 1 ml of PBS, pH 7.2 and homogenized by hand using a Duall glass-glass homogenizer. The sample was further homogenized following the addition of 1 ml chloroform/methanol (2:1, v/v). The sample was transferred to a boroscilicate tube and lipids were extracted into 4 mls of chloroform. The organic extract was washed with 3 mls PBS, pH 7.2 and the samples were then centrifuged at 3,000×g, 10 min. The choloroform phase was decanted and the aqueous phase was re-extracted with another 4 mls of chloroform. Following centrifugation, the chloroform phases were combined and the samples were taken to dryness under nitrogen gas. Samples residues were resuspended in 100 μl hexane and analyzed by HPLC as described below.

HPLC Analysis. Chromatographic separations were achieved on an Agilent Zorbax Rx-Sil Column (5 μm, 4.6×250 mm) using an Agilent 1100 series liquid chromatograph equipped with fluorescence and diode array detectors. The mobile phase (hexane/2-propanol/ethanol/25 mM KH₂PO₄, pH 7.0/acetic acid; 485/376/100/5010.275, v/v) was delivered at 1 ml/min. Sample peak identification was made by comparison to retention time and absorbance spectra of authentic standards. Data are reported as peak fluorescence (L.U.) obtained from the fluorescence detector.

The following examples provide illustrative methods for testing the effectiveness and safety of the Megalin-modulating agents. These examples are provided for illustrative purposes only and not to limit the scope of the claims provided herein.

### Example 1

### Effect of a Megalin-Modulating Agent on A2E Accumulation

Administration of a Megalin-modulating agent to an experimental group of mice and administration of DMSO alone to a control group of mice is performed and assayed for accumulation of A2E. The experimental group is given 2.5 to 20 mg/kg of the Megalin-modulating agent per day in 10 to 25 μl of DMSO. Higher dosages are tested if no effect is seen with the highest dose of 50 mg/kg. The control group is given 10 to 25 μl injections of DMSO alone. Mice are administered either experimental or control substances by intraperitoneal (i.p.) injection for various experimental time periods not to exceed one month.

To assay for the accumulation of A2E in abca4⁻/⁻ mice RPE, 2.5 to 20 mg/kg of a Megalin-modulating agent is provided by i.p. injection per day to 2-month old abca4⁻/⁻ mice. After 1 month, both experimental and control mice are killed and the levels of A2E in the RPE are determined by HPLC. In addition, the autofluorescence or absorption spectra of N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, and/or N-retinylidene-phosphatidylethanolamine may be monitored using a UV/V is spectrometer.

### Example 2

### Effect of a Megalin-Modulating Agent on Lipofuscin Accumulation

Administration of a Megalin-modulating agent to an experimental group of mice and administration of DMSO alone to a control group of mice is performed and assayed for the accumulation of lipofuscin. The experimental group is given 2.5 to 20 mg/kg of the Megalin-modulating agent per day in 10 to 25 μl of DMSO. Higher dosages are tested if no effect is seen with the highest dose of 50 mg/kg. The control group are given 10 to 25 μl injections of DMSO alone. Mice are administered either experimental or control substances by i.p. injection for various experimental time periods not to exceed one month. Alternatively, mice can be implanted with a pump which delivers either experimental or control substances at a rate of 0.25 μl/hr for various experimental time periods not to exceed one month.

To assay for the effects of the Megalin-modulating agent on the formation of lipofuscin in treated and untreated abca4⁻/⁻ mice, eyes can be examined by electron or fluorescence microscopy.

### Example 3

### Effect of a Megalin-Modulating Agent on Rod Cell Death or Rod Functional Impairment

Administration of a Megalin-modulating agent to an experimental group of mice and administration of DMSO alone to a control group of mice is performed and assayed for the effects of a Megalin-modulating agent on rod cell death or rod functional impairment. The experimental group is given 2.5 to 20 mg/kg of the Megalin-modulating agent per day in 10 to 25 μl of DMSO. Higher dosages are tested if no effect is seen with the highest dose of 50 mg/kg. The control group is given 10 to 25 μl injections of DMSO alone. Mice are administered either experimental or control substances by i.p. injection for various experimental time periods not to exceed one month. Alternatively, mice can be implanted with a pump which delivers either experimental or control substances at a rate of 0.25 μl/hr for various experimental time periods not to exceed one month.

Mice that are treated to 2.5 to 20 mg/kg of a Megalin-modulating agent per day for approximately 8 weeks can be assayed for the effects of the Megalin-modulating agent on rod cell death or rod functional impairment by monitoring ERG recordings and performing retinal histology.

### Example 4

### Testing for Protection from Light Damage

The following study is adapted from Sieving, P. A., et al, Proc. Natl. Acad. Sci., 98:1835-40 (2001). For chronic light-exposure studies, Sprague-Dawley male 7-wk-old albino rats are housed in 12:12 h light/dark cycle of 5 lux fluorescent white light. Injections of 20-50 mg/kg a Megalin-modulating agent by i.p. injection in 0.18 ml DMSO are given three times daily to chronic rats for 8 wk. Controls receive 0.18 ml DMSO by i.p. injection. Rats are killed 2 d after final injections. Higher dosages are tested if no effect is seen with the highest dose of 50 mg/kg.

For acute light-exposure studies, rats are dark-adapted overnight and given a single i.p. injection of the Megalin-modulating agent 20-50 mg/kg in 0.18 ml DMSO under dim red light and kept in darkness for 1 h before being exposed to the bleaching light before ERG measurements. Rats exposed to 2,000 lux white fluorescent light for 48 h. ERGs are recorded 7 d later, and histology is performed immediately.

Rats are euthanized and eyes are removed. Column cell counts of outer nuclear layer thickness and rod outer segment (ROS) length are measured every 200 μm across both hemispheres, and the numbers are averaged to obtain a measure of cellular changes across the entire retina. ERGs are recorded from chronic rats at 4 and 8 wks of treatment. In acute rodents, rod recovery from bleaching light is tracked by dark-adapted ERGs by using stimuli that elicit no cone contribution. Cone recovery is tracked with photopic ERGs. Prior to ERGs, animals are prepared in dim red light and anaesthetized. Pupils are dilated and ERGs are recorded from both eyes simultaneously by using gold-wire corneal loops.

### Example 5

### Combination Therapy Involving a Megalin-Modulating Agent and Fenretinide

Mice and/or rats are tested in the manner described in Examples 1-4, but with an additional two arms. In one of the additional arms, groups of mice and/or rats are treated with increasing doses of fenretinide, from 5 mg/kg per day to 50 mg/kg per day. In the second additional arm, groups of mice and/or rats are treated with a combination of 20 mg/kg per day of a Megalin-modulating agent and increasing doses of fenretinide, from 5 mg/kg per day to 50 mg/kg per day. The benefits of the combination therapy are assayed as described in Examples 1-4.

### Example 6

### Effect of a Megalin-Modulating Agent on Retinol and RBP Levels in RPE Cells

Mice and/or rats are tested in the manner described in Examples 1-4, however the levels of retinol (or retinyl esters) and RBP in the RPE cells are determined by HPLC analysis. This experiment determines the amount of modulating activity that is attributed to the agent in question. Direct comparison of retinol and RBP levels in the RPE cells between the experimental group and the control group provides a direct correlation to agents that are responsible for inhibiting the binding and uptake of retinol, retinol-RBP or retinol-RBP-TTR to members of LDL receptor gene family that are expressed in the RPE cells.

All of the methods disclosed and claimed herein can be made and executed without undue experimentation in light of the present disclosure. It will be apparent to those of skill in the art that variations may be applied to the methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

I claim:

1. A method for treating an ophthalmic condition in an eye of a mammal comprising,
administering to the mammal an effective amount of an agent that modulates the activity of a member of the LDL receptor gene family in the retina and/or retinal pigment epithelium cells in the eye of the mammal.

2. The method of claim 1, wherein the member of the LDL receptor gene family is megalin or a megalin-related protein.

3. The method of claim 1, wherein the member of the LDL receptor gene family is a retinoid binding protein receptor.

4. The method of claim 1, wherein the activity of the member of the LDL receptor gene family is the binding of the member of the LDL receptor gene family to a second agent selected from the group consisting of:
vitamin-binding proteins, lipoproteins, immune- and stress related proteins, steroid hormone binding proteins, Hormones and precursors, peptides, enzyme and enzyme inhibitors, albumin, lactoferrin, hemoglobin, odorant-binding protein, transthyretin; polybasic drugs and toxins, RAP, calcium (Ca²⁺), and cytochrome c.

5. The method of claim 1, wherein the activity of the member of the LDL receptor gene family is the binding of the member of the LDL receptor gene family to a second agent selected from the group consisting of:
retinol, a retinol-RBP complex, a retinol-RBP-TTR complex, an interphotoreceptor retinoid binding protein (IRBP), a retinol-IRBP complex, transcobalamin-vitamin B12, transcobalamin-vitamin B12 binding protein, vitamin-D-binding protein, apolipoprotein B, apolipoprotein E, apolipoprotein J/clusterin, apolipoprotein H; immunoglobulin light chains, PAP-1, β2-microglobulin; sex hormone binding protein-estrogens, androgen binding protein-androgens; parathyroid hormone, insulin, epidermal growth factor, prolactin, thyroglobulin; plasminogen activator inhibitor-1 (PAI-1), urokinase-PAI-1, tPA-PAI-1, pro-urokinase, lipoprotein lipase, plasminogen, β-amylase, β1-microglobulin, lysozyme; albumin, lactoferrin, hemoglobin, odorant-binding protein, transthyretin; aminoglycosides, polymyxin B, aprotinin, trichosanthin, gentamicin; RAP, Ca²⁺, and cytochrome c.

6. The method of claim 1, wherein the activity of the member of the LDL receptor gene family is the binding of the member of the LDL receptor gene family to retinol, a retinol-RBP complex, or a retinol-RBP-TTR complex.

7. The method of claim 1, wherein the activity of the LDL receptor gene family member is the binding of the member of the LDL receptor gene family to retinoid binding proteins.

8. The method of claim 1, wherein the activity of the member of the LDL receptor gene family is the trancytosis of a second agent selected from the group consisting of:
vitamin-binding proteins, lipoproteins, immune- and stress related proteins, steroid hormone binding proteins, hormones and precursors, peptides, enzyme and enzyme inhibitors, albumin, lactoferrin, hemoglobin, odorant-binding protein, transthyretin; polybasic drugs and toxins, RAP, calcium (Ca 2+), and cytochrome c.

9. The method of claim 1, wherein the activity of the member of the LDL receptor gene family is the transcytosis of a second agent selected from the group consisting of:
retinol, a retinol-RBP complex, a retinol-RBP-TTR complex, an interphotoreceptor retinoid binding protein (IRBP), a retinol-IRBP complex, transcobalamin-vitamin B12, transcobalamin-vitamin B12 binding protein, vitamin-D-binding protein, apolipoprotein B, apolipoprotein E, apolipoprotein J/clusterin, apolipoprotein H; immunoglobulin light chains, PAP-1, β2-microglobulin; sex hormone binding protein-estrogens, androgen binding protein-androgens; parathyroid hormone, insulin, epidermal growth factor, prolactin, thyroglobulin; plasminogen activator inhibitor-1 (PAI-1), urokinase-PAI-1, tPA-PAI-1, pro-urokinase, lipoprotein lipase, plasminogen, β-amylase, β1-microglobulin, lysozyme; albumin, lactoferrin, hemoglobin, odorant-binding protein, transthyretin; aminoglycosides, polymyxin B, aprotinin, trichosanthin, gentamicin; RAP, Ca²⁺, and cytochrome c.

10. The method of claim 1, wherein the agent binds retinol-binding protein.

11. The method of claim 1, wherein the agent binds to transthyretin.

12. The method of claim 1, wherein the agent binds to interphotoreceptor retinoid binding protein (IRBP).

13. The method of claim 1, wherein the agent modulates the expression of the member of the LDL receptor gene family in the retina and/or retinal pigment epithelium cells.

14. The method of claim 1, wherein the agent is selected from the group consisting of an antibody, a polypeptide, a nucleic acid, a polynucleic acid, a polymer, receptor associated protein (RAP) or fragments thereof, a low molecular weight organic compound, vitamin-binding proteins, lipoproteins, immune- and stress related proteins, steroid hormone binding proteins, hormones and precursors, peptides, enzyme and enzyme inhibitors, albumin, lactoferrin, hemoglobin, odorant-binding protein, transthyretin; polybasic drugs and toxins, RAP, calcium (Ca 2+), calcium scavengers, reducing agents and cytochrome c.

15. The method of claim 1, wherein the agent is selected from the group consisting of an antibody, a polypeptide, a nucleic acid, a polynucleic acid, a polymer, receptor associated protein (RAP) or fragments thereof, a low molecular weight organic compound, retinol, a retinol-RBP complex, a retinol-RBP-TTR complex, an interphotoreceptor retinoid binding protein (IRBP), a retinol-IRBP complex, transcobalamin-vitamin B12, transcobalamin-vitamin B12 binding protein, vitamin-D-binding protein, apolipoprotein B, apolipoprotein E, apolipoprotein J/clusterin, apolipoprotein H; immunoglobulin light chains, PAP-1, β2-microglobulin; sex hormone binding protein-estrogens, androgen binding protein-androgens; parathyroid hormone, insulin, epidermal growth factor, prolactin, thyroglobulin; plasminogen activator inhibitor-1 (PAI-1), urokinase-PAI-1, tPA-PAI-1, pro-urokinase, lipoprotein lipase, plasminogen, β-amylase, β1-microglobulin, lysozyme; albumin, lactoferrin, hemoglobin, odorant-binding protein, transthyretin; aminoglycosides, polymyxin B, aprotinin, trichosanthin, gentamicin; RAP, RAP fragments, Ca 2+, calcium scavengers, reducing agents and cytochrome c.

16. The method of claim 1, further comprising repeating administrations of the effective amount of the agent.

17. The method of claim 16, wherein at least one time between administrations is at least one week.

18. The method of claim 16, wherein at least one time between administrations is at least one day.

19. The method of claim 1, further comprising administering at least one additional agent selected from the group consisting of an inducer of nitric oxide production, an anti-inflammatory agent, a physiologically acceptable antioxidant, a physiologically acceptable mineral, a negatively charged phospholipid, a carotenoid, a statin, an anti-angiogenic drug, a matrix metalloproteinase inhibitor, 13-cis-retinoic acid, or a compound having the structure of Formula (A): 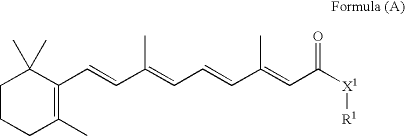
wherein
X₁ is selected from the group consisting of NR², O, S, CHR²;
R¹ is (CHR²)ₓ-L¹-R³, wherein
x is 0, 1, 2, or 3; L¹ is a single bond or —C(O)—;
R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and
R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle.

20. The method of claim 19, wherein the compound having the structure of Formula (A) is 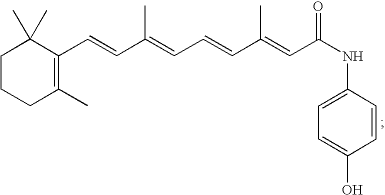
or an active metabolite, or a pharmaceutically acceptable prodrug or solvate thereof.

21. The method of claim 19, wherein the compound is 4-hydroxyphenylretinamide; 4-methoxyphenylretinamide; or a metabolite, or a pharmaceutically acceptable prodrug or solvate thereof.

22. The method of claim 1, further comprising administering to the mammal a therapy selected from the group consisting of extracorporeal rheopheresis, limited retinal translocation, photodynamic therapy, drusen lasering, macular hole surgery, macular translocation surgery, Phi-Motion, Proton Beam Therapy, Retinal Detachment and Vitreous Surgery, Scleral Buckle, Submacular Surgery, Transpupillary Thermotherapy, Photosystem I therapy, MicroCurrent Stimulation, RNA interference, administration of eye medications such as phospholine iodide or echothiophate or carbonic anhydrase inhibitors, microchip implantation, stem cell therapy, gene replacement therapy, ribozyme gene therapy, photoreceptor/retinal cells transplantation, laser photocoagulation, and acupuncture.

23. The method of claim 1, further comprising an additional treatment for retinal degeneration.

24. The method of claim 1, wherein the mammal is a human.

25. The method of claim 24, wherein the human has an ophthalmic condition or trait selected from the group consisting of Stargardt Disease, recessive retinitis pigmentosa, recessive cone-rod dystrophy, dry-form age-related macular degeneration, exudative age-related macular degeneration, cone-rod dystrophy, retinitis pigmentosa, a lipofuscin-based retinal degeneration, photoreceptor degeneration, and geographic atrophy.


SEQUENCE LISTING (19 sequences)

SEQ ID NO: 1 - protein, 4 aa, Artificial Sequence
  Description of Artificial Sequence Synthetic peptide
FWTD

SEQ ID NO: 2 - protein, 4 aa, Artificial Sequence
  Description of Artificial Sequence Synthetic peptide
YWTD

SEQ ID NO: 3 - protein, 10 aa, Rattus norvegicus
DLLVGDLHGR

SEQ ID NO: 4 - protein, 7 aa, Rattus norvegicus
LYLVETK

SEQ ID NO: 5 - protein, 11 aa, Rattus norvegicus
IDVVNLEGNQR

SEQ ID NO: 6 - protein, 12 aa, Rattus norvegicus
VTLITENLGHPR

SEQ ID NO: 7 - protein, 8 aa, Rattus norvegicus
NFLLFSSK

SEQ ID NO: 8 - protein, 17 aa, Rattus norvegicus
LGHVDQMTHPFGLTVFK

SEQ ID NO: 9 - protein, 14 aa, Rattus norvegicus
VTGSENPLLVVASR

SEQ ID NO: 10 - protein, 10 aa, Rattus norvegicus
VFWSDLLQGK

SEQ ID NO: 11 - protein, 7 aa, Rattus norvegicus
GLALDPR

SEQ ID NO: 12 - protein, 14 aa, Rattus norvegicus
DNIIFGISLDPEVK

SEQ ID NO: 13 - protein, 8 aa, Rattus norvegicus
SIEVLTLK

SEQ ID NO: 14 - protein, 12 aa, Rattus norvegicus
TVLVSEGIVTPR

SEQ ID NO: 15 - protein, 13 aa, Rattus norvegicus
LPTQPSGISTVVK

SEQ ID NO: 16 - protein, 14 aa, Rattus norvegicus
QNSNIEPYLIFSNR

SEQ ID NO: 17 - protein, 8 aa, Rattus norvegicus
LYWIDAEK

SEQ ID NO: 18 - protein, 19 aa, Rattus norvegicus
KWLITTQLDQPAAIAVNPK

SEQ ID NO: 19 - protein, 10 aa, Rattus norvegicus
FWTDQGKQPK